SEQUENCE LISTING (67 sequences)

SEQ ID NO: 1 - DNA, 32 bp, synthetic construct
  source 1..32: mol_type = other DNA
catgcatgccgggcatcacgtgtgtggatatc

SEQ ID NO: 2 - DNA, 30 bp, synthetic construct
  source 1..30: mol_type = other DNA
catagatcttcacagaccgctcagcttgcg

SEQ ID NO: 3 - DNA, 36 bp, synthetic construct
  source 1..36: mol_type = other DNA
catggtaccaggaggaattaaatggataaatttcgt

SEQ ID NO: 4 - DNA, 32 bp, synthetic construct
  source 1..32: mol_type = other DNA
ccggaattccgctgaccttgtccatcacgata

SEQ ID NO: 5 - DNA, 32 bp, synthetic construct
  source 1..32: mol_type = other DNA
catcccgggcgctgaccttgtccatcacgata

SEQ ID NO: 6 - DNA, 21 bp, synthetic construct
  source 1..21: mol_type = other DNA
ataacctttcattcccagcgg

SEQ ID NO: 7 - DNA, 20 bp, synthetic construct
  source 1..20: mol_type = other DNA
gagaaacggctaccacatcc

SEQ ID NO: 8 - DNA, 22 bp, synthetic construct
  source 1..22: mol_type = other DNA
gatacgctcattccgattacag

SEQ ID NO: 9 - DNA, 21 bp, synthetic construct
  source 1..21: mol_type = other DNA
ggggatttttgcacagttcgg

SEQ ID NO: 10 - DNA, 21 bp, synthetic construct
  source 1..21: mol_type = other DNA
tccacaaggtgatgctgttca

SEQ ID NO: 11 - DNA, 21 bp, synthetic construct
  source 1..21: mol_type = other DNA
tgaacatcggtgttgttggtc

SEQ ID NO: 12 - DNA, 22 bp, synthetic construct
  source 1..22: mol_type = other DNA
tcctgttcagtgaaggtgactt

SEQ ID NO: 13 - DNA, 24 bp, synthetic construct
  source 1..24: mol_type = other DNA
tcgtacagcaagactaagaaatca

SEQ ID NO: 14 - DNA, 23 bp, synthetic construct
  source 1..23: mol_type = other DNA
tttgattgttgttgggactgtct

SEQ ID NO: 15 - DNA, 23 bp, synthetic construct
  source 1..23: mol_type = other DNA
cttaagaacagagacacaccgga

SEQ ID NO: 16 - DNA, 22 bp, synthetic construct
  source 1..22: mol_type = other DNA
acacgctttggatttttcactg

SEQ ID NO: 17 - DNA, 22 bp, synthetic construct
  source 1..22: mol_type = other DNA
tgctggaaaggtactttgacag

SEQ ID NO: 18 - DNA, 23 bp, synthetic construct
  source 1..23: mol_type = other DNA
gcacttcaaattgatcaccatgc

SEQ ID NO: 19 - DNA, 21 bp, synthetic construct
  source 1..21: mol_type = other DNA
gctgcccttgatcatctttgc

SEQ ID NO: 20 - DNA, 22 bp, synthetic construct
  source 1..22: mol_type = other DNA
ggatccaggcagaccttcattc

SEQ ID NO: 21 - DNA, 24 bp, synthetic construct
  source 1..24: mol_type = other DNA
tactggacatgagattcacaacct

SEQ ID NO: 22 - DNA, 22 bp, synthetic construct
  source 1..22: mol_type = other DNA
ctttcacactttcctggagttc

SEQ ID NO: 23 - DNA, 23 bp, synthetic construct
  source 1..23: mol_type = other DNA
cactgaagcacagatcaaacacc

SEQ ID NO: 24 - DNA, 24 bp, synthetic construct
  source 1..24: mol_type = other DNA
ctgttctgtacactcctaggttgg

SEQ ID NO: 25 - DNA, 23 bp, synthetic construct
  source 1..23: mol_type = other DNA
tcttcaggagtttggagaacctg

SEQ ID NO: 26 - DNA, 23 bp, synthetic construct
  source 1..23: mol_type = other DNA
cagtttcaagcctgaagagaagg

SEQ ID NO: 27 - DNA, 22 bp, synthetic construct
  source 1..22: mol_type = other DNA
gaaacatctggagtcgagctgc

SEQ ID NO: 28 - DNA, 22 bp, synthetic construct
  source 1..22: mol_type = other DNA
gacatttttgcgggctgaagtc

SEQ ID NO: 29 - DNA, 1374 bp, unidentified
  source 1..1374: mol_type = genomic DNA
atgagtcaatttgagaagtcattcaagggcagaactgaggtcacaataaccgagtatcgctctcatactgtcaaagatgtgcacagaagcttacttacggctgacaaatctctaaggaagtcattctgctttaggaacgccctaaaccagttcttggataaagatttgcctcttttgcccattcggccaaaattagagtccagggttgctgtgaaaaagtctaagctgagaagtcaactgtcgttcagacctggtttaactcaggaggaagcaattgacctttacaacaagggctatgatggtgacagtgtctcaggtgccttacaagatagagtagtcaatgagcctgtagcttactcgagtgcagataatgacaagtttcacaggggcttggcagctttagggtacactttggctgatagagcacttgatacgtgcgaatccggcttcgtgagagcaattcctaccactccatgtgggttcatatgttgtgggccaggttctttcaaagattcacttgggtttgtgataaaaatcggcgagttctggcacatgtatgacgggttccagcacttcgtcgctgtcgaggatgctaagtttctagcaagtaagtctccttcgttttggttagcaaaacgtcttgcaaagaggctgaatctggtcccaaaagaggatccatctatagcagcagctgagtgcccttgtaaaaaagtgtgggaagctagttttgctagggcacctactgcactagatccatttggaggcagggccttttgcgaccagggttgggtgtaccacagagacgtcgggtatgcaacagccaaccacatatcacaggagacactttttcaacaagcgctttcagtgaggaaccttgggccacaaggtagcgcaaatgtctcaggctcaatacataccgccctggacaggctcagagcagcgtacagtaggggaacgcctgcctctagatctattctgcaagggcttgcgaatctcatcacacctgtgggtgaaaactttgaatgtgatctcgacaggaggaaactcaatataaaggcattacgttctcccgagaggtacattacgatagagggcctggttgtaaacctagacgatgtggtgagagggttctaccttgacaaggcgaaggtcactgttctctcgagatcaaggtggatgggttacgaggacctacctcagaaacctccgaatggtacattttactgcagaaagaggaaggcaatgcttctcatctcatgtagcccaggcacgtatgcaaagaagcgaaaagtggcagtgcaggaggatcgctttaaggatatgagggttgagaatttccgggaggtagcggaaaatatggatctaaatcagtag

SEQ ID NO: 30 - DNA, 1260 bp, unidentified
  source 1..1260: mol_type = genomic DNA
atggactcgcggtttgcacagctaactggggttttctgtgatgatttcacttatagcgaagggagtcgaaggttcctaagttcctacagtacagtagagagacgtccaggggtccccgtagagggtgactgctatgactgtttgaagaataagtggattgcctttgagttggaaggccagccgcggaagtttccaaaggcaacagtccgttgcatcttgaacaatgatgccacatacgtttgctctgagcaagagtaccagcagatttgtaaagtacaattcaaggattatttagagattgacggggttgttaaagttgggcacaaggcatcctacgatgctgagctaagggaacggctattggaactgccacatccaaagagtggcccgaagcctcatatcgagtgggtggcaccacccagacttgcggacatatccaaggagacagctgagctaaagaggcagtatgggttcttcgagtgctcaaagttcctcgcctgcggtgaggagtgtggccttgaccaagaggcaagagaactcatactgaacgagtacgcacgcgatagagagtttgagttccgcaacggagggtggatacaaaggtacacagttgcttctcataagcctgctacacagaagatattgcctctaccagctagtgccccacttgctcgtgagcttttgatgttgatagctagaagtacaactcaggcagggaaagtactgcatagcgataataccagcatactagctgtaccggtcatgcgtgactctggaaagcacagtaaaaggagaccaaccgcctccactcaccacttagttgtgggtctaagtaaacctggttgtgagcacgattttgagtttgacgggtatagggcagctgtgcatgtaatgcacttagatcctaaacaatcggctaatataggggagcaggactttgtgagtacccgagaaatttacaagctggatatgttggaactacctcccataaataagaagggtgatctggacagagctagtggtcttgagacaagatgggatgtcatcttacttctggaatgcctcgactctacaagggttagccaagcagtggctcaacattttaataggcatcgactggcacttagtgtctgcaaggacgagttcaggaaaggctaccaactagctccggagataaggggtacaatacccttaagctcactttattattcgctttgtgcagtaagattgcggatgacagtacacccatttgcgagatga

SEQ ID NO: 31 - DNA, 1065 bp, unidentified
  source 1..1065: mol_type = genomic DNA
atggtgagaactacaaagactagtatggcagctgccagcactgttgcaccagaggtagcaatggatgaaggttcacccagcacttcgcaggcacaagttgaactcccaagaaaccttgaggttttcaatgaagcttgtggtcatgtgtttggaagttcctttaacagggaggacaacagtgtgatatctgatgctgctgcattcctttttaaaatgcacacccactcccttgatggtcaagaggctaaggttctgagagccagtgaaaagaagagagagagggagaacgctaagaaatcaaggaaggcaccagaagcagggatgagggtcggaaggagccttatattaaccagcagatggactgaatactgcgcaacctgtgtacctgcactgggttcaaagatgaaggtgataaaagcctcaggggacgcggctatgattcagatgatgaaggaccataactctctattgagagtgtgtgttcgcattgaggtctggaaggctaggtacgtcagtttggttgctctcgatgagaggattcagactctggaggatgctcagtggttcccctacctgagtggggattcctatcgtgcttgcccagggctggttggtggctactttgcaaagaaagcagcagcaggagaaagaggaaagaactacaaaaagttgaatcagactgctataatcccgcctccgagatttctaatcattggccacaggctgcagataggtgaccaggtcaccctcagggagctgcttgcctcaattgcttggggcctttgcgacggtgtccttgctgagtgttggagcccctcgcagggggacgggagtattggtgtagttgttggtctacctctgcaggctacaggaagctgtttcctggtggtagctagccacgggctctcagcaattgccgactccaggattgagggaacagggaacacgaatctgctagaagagtgcattgctattcagaaacaggatggcgtcataaaatgtaagagaagtgggaagagtctgtatcactgcctcaaagagacagcaggggctgttggaagatag

SEQ ID NO: 32 - DNA, 1032 bp, unidentified
  source 1..1032: mol_type = genomic DNA
atgtttctcttatctcagactccaatagctatgcaggtgctagtcctgacaagctgcctagtttgcgcactagcaagtgatgaaagtttaaggataaagcgactacaatcgtacctaaacaatacctaccaaagtagggagatagaaagtgagataaggcgtggatttgcatccaagttcaggatggagagttgctcctgcaccatgggggtgcactacattgtaaccccatcctcgggtgggtcgttctgcactgggttacatgcagtacctgacagcttcccagccctcgggtacaaacttcccaaagcagggggaagaggtgattggaaggctactgaagtcagggttgacgaagatagtggagttgttctatacaacgtttccaggtgtagccacagtaccgattgcagagatttggaggtgtattccaccgtactgccaggtcagtgtgagtgtaccagacccactgttgacgactacaagaccatgctggcttcaaggcagccgaagtcgtttgtagtagcaggcctcattatactgtgtttactcgctagctcagtagcaattggcatgggtgtttacaattatgctggggtcatcggcctagcggacgcagctcaagcagatgtttctgagatttgggagtatttagaagctttgacacgggaagtcaccggcatgacgctaggagagttttgctcgattaaatctctcgtctgtaaatctgataacataggcaaattcaaggagcaatttgcagcctttggggaagccattcttgcaatagtgtttgggatgctagagaaatataagtttgtctattacctggtgctttcgttgatggttctctcgttgctcagcaaacttatttctctgttgaagcaggtgcccttctatgggagtatcaaagttttagtattccggaggctaagagttgtgtgtttcaagacctttttctttattaagaagcggcttaagaagaaaggcccgcttgaggatgacgaagtccctctgcttccattatcttga

SEQ ID NO: 33 - DNA, 954 bp, unidentified
  source 1..954: mol_type = genomic DNA
atgcatttttatctacaggattgtccaatgagttggcttagggtgataagaacccttactttgttttcaacactcttcagcagaagcgatcagtgcgtggacaacatgtggcgtttttacgggagatcaaactacacttcaagcgtagttattgacggggataaatattctgttgaaggttcatattcgagtagtgattacctggatccagcagtactgaaggtagttctgggacttgatggtagcaacgaagtcatagactcaggcggatctccatactacaggtatgatttggaaggatcaaagggggaactccatcatctgaactgcaactttgttgagaaacgatgtaatccgacgctgaactttatgcttggaggatttgttctgtgcccgggaatatcgagaaaggaactggagcctgtaaccgacaagatattggagagccggggaataccgggtcgaggcaaaatacatactataaaaataagttctaaactgtttgagacatcgctgtgcctttcgaagaggaggcccatctttagcacctgtatgctaatgtcgcgtggtctctgtacaaactgtaagcgtactatagatagaacatatatgacatcaaacggcttcagaactgaatacaagtggagctgcagagacaatagcacaaaacagtgttgggtattagttgagtctctggaggagaatcactccccgtacaagtgccacttttccgcagtggaagtcctactaccagccgaaataaaacgtcaccagctcatcagcgagtggtctgcgaagcaggatgaagttgcttataagaaatcagatgcttatcttcttgctcgtacttttctcagctatacaaaaatgcgcagattgaatcctgtaatcgatctttcgatatcaccaccagtaacagtaagatcctgctgtaaaatcaataaatacatgtga

SEQ ID NO: 34 - DNA, 588 bp, unidentified
  source 1..588: mol_type = genomic DNA
atgtcctacaagattggtgagcttgagagaattatcacgcgtaaaaacaccctccccaaggacagcggaagtcagactgggctgttccatcgactgctcttagagcattactctggtgcctcgaacgtatggttcttttgtgcaactgggttcacacccaacacaaatggcacaacctggattgtgttgacgagtcacccaaccgatggtggagaaaaggtacctctgaaatggaagtatgaagtgagccccggattaccagtcagaagggtacttgcccaggaaggtacagcagtaaggggtccgaaaggagcctacttagtcaaaggggacatgcgtctctgtccaactaccttctataccagaagggaagcgaagtactggctctgtgcgccatctccaaagtttccacactggaccaagagatcagcgctggtgaccagcactcgaccactgactgagttgagcagggttgccacatacctagaggctataagtaagggtgcaactgatgttaatgaatcgtggtgttcctaccacagagttgggttagtgccaatccctggagggatcacttttgaactctaa

SEQ ID NO: 35 - DNA, 525 bp, unidentified
  source 1..525: mol_type = genomic DNA
atggctcaaattccaacactaagagagggccaagggaagctctacgatttcacgctcaacggtatgacagtgactagggacacagtcaacactgtagttgctctagagtttcttgtcaatgcaagctcggatttgctttccctaacaattggcgaaggcctctcagaagaaacaaagtttaaacacctgcttgttaagcacgctggtatgacccgtaagcggatagaggaaaggctgggacgaatctcgaggcgagtcagtgtgacagtcgacgcaattataataacaaaccgcaagggtcaaagatttgaattcaatcggaaacagtacctggatattgccaaacaagctatgaagcttaagctccctgggattaactgcgtcgacatacccactgcgctcgcttttctcgaggaagtcctggcaactgctttgaaggacactgaaggttcacaagatgacagaatggcccttaaggcagacacttctgctgctatcaatcatttccgtgaaatgcttaaataa

SEQ ID NO: 36 - DNA, 351 bp, unidentified
  source 1..351: mol_type = genomic DNA
atgtcacgatggatagaaaatacagattctgtgtcagtaatcttgacagagatgagtcggtcgtacgtcactttgtgccattaccccccttggagcttgtgctgcggcggcaagacatcacaacctggtcaagtttggatcctggatcgaaaacattgtctagaatgttcagagatctcagagttaatgacactgggtcagccaacttggcaggagagtgcaatggtgatagggagctgggtccaagtagtaacggagcacgggattttacacacttcaacatcggaaaggcaggcgccaagaagggtcatgtggaggatatctgacatggctggcgataaaactttatga

SEQ ID NO: 37 - DNA, 342 bp, unidentified
  source 1..342: mol_type = genomic DNA
atgagtgtggcagattatttgtcaagtgacagcgattcggaggctgagagctcaggctgtttagtactaagaagtcggaagatcaagaagggcaagaaagctgcttcaaagaagcgaagttggaagaatgaaaggtatggtgctgacgagagcggtgaagataatatagagtggggtgacgaagtcgacctcgagatggacgactgtgattctgcaatcccagagtgggctagggttgattttaatcccaaaaacagaagggacagagaggatgatgggcagagtgacctatctcgattttccgaagatttcggaaagaagtctctcgacgtgcagtcttag

SEQ ID NO: 38 - DNA, 1374 bp, synthetic construct
  source 1..1374: mol_type = other DNA
atgagccagttcgaaaaaagcttcaaaggccgtaccgaagttaccatcaccgaataccgtagccacaccgttaaagacgttcaccgtagcctgctgaccgctgacaaaagcctgcgtaaaagcttctgcttccgtaacgctctgaaccagttcctggacaaagacctgccgctgctgccgatccgtccgaaactggaaagccgtgttgctgttaaaaaaagcaaactgcgtagccagctgagcttccgtccgggcctgacccaggaagaagctatcgacctgtacaacaaaggctacgacggcgacagcgttagcggcgctctgcaggaccgtgttgttaacgaaccggttgcttacagcagcgctgacaacgacaaattccaccgtggcctggctgctctgggctacaccctggctgaccgtgctctggacacctgcgaaagcggcttcgttcgtgctatcccgaccaccccgtgcggcttcatctgctgcggcccgggcagcttcaaagacagcctgggcttcgttatcaaaatcggcgaattctggcacatgtacgacggcttccagcacttcgttgctgttgaagacgctaaattcctggctagcaaaagcccgagcttctggctggctaaacgtctggctaaacgtctgaacctggttccgaaagaagacccgagcatcgctgctgctgaatgcccgtgcaaaaaagtttgggaagctagcttcgctcgtgctccgaccgctctggacccgttcggcggccgtgctttctgcgaccagggctgggtttaccaccgtgacgttggctacgctaccgctaaccacatcagccaggaaaccctgttccagcaggctctgagcgttcgtaacctgggcccgcagggcagcgctaacgttagcggcagcatccacaccgctctggaccgtctgcgtcgtcgttacagccgtggcaccccggctagccgtagcatcctgcagggcctgcgtaacctgatcaccccggttggcgaaaacttcgaatgcgacctggaccgtcgtaaactgaacatcaaagctctgcgtagcccggaacgttacatcaccatcgaaggcctggttgttaacctggacgacgttgttcgtggcttctacctggacaaagctaaagttaccgttctgagccgtagccgttggatgggctacgaagacctgccgcagaaaccgccgaacggcaccttctactgccgtaaacgtaaagctatgctgctgatcagctgcagcccgggcacctacgctaaaaaacgtaaagttgctgttcaggaagaccgtttcaaagacatgcgtgttgaaaacttccgtgaagttgctgaaaacatggacctgaaccagtaa

SEQ ID NO: 39 - DNA, 1260 bp, synthetic construct
  source 1..1260: mol_type = other DNA
atggacacgcgtttcgctcagctgaccggcgttttctgcgacgacttcacctacagcgaaggcagccgtcgtttcctgagcagctacagcaccgttgaacgtcgtccgggcgttccggttgaaggcgactgctacgactgcctgaaaaacaaatggatcgctttcgaactggaaggccagccgcgtaaattcccgaaagctaccgttcgttgcatcctgaacaacgacgctacctacgtttgcagcgaacaggaataccagcagatctgcaaagttcagttcaaagactacctggaaatcgacggcgttgttaaagttggccacaaagctagctacgacgctgaactgcgtgaacgtctgctggaactgccgcacccgaaaagcggcccgaaaccgcacatcgaatgggttgctccgccgcgtctggctgacatcagcaaagaaaccgctgaactgaaacgtcagtacggcttcttcgaatgcagcaaattcctggcttgcggcgaagaatgcggcctggaccaggaagctcgtgaactgatcctgaacgaatacgctcgtgaccgtgaattcgaattccgtaacggcggctggatccagcgttacaccgttgctagccacaaaccggctacccagaaaatcctgccgctgccggctagcgctccgctggctcgtgaactgctgatgctgatcgctcgtagcaccacccaggctggcaaagttctgcacagcgacaacaccagcatcctggctgttccggttatgcgtgacagcggcaaacacagcaaacgtcgtccgaccgctagcacccaccacctggttgttggcctgagcaaaccgggctgcgaacacgacttcgaattcgacggctaccgtgctgctgttcacgttatgcacctggacccgaaacagagcgctaacatcggcgaacaggacttcgttagcacccgtgaaatctacaaactggacatgctggaactgccgccgatcaacaaaaaaggcgacctggaccgtgctagcggcctggaaacccgttgggacgttatcctgctgctggaatgcctggacagcacccgtgttagccaggctgttgctcagcacttcaaccgtcaccgtctggctctgagcgtttgcaaagacgaattccgtaaaggctaccagctggctccggaaatccgtggcaccatcccgctgagcagcctgtactacagcctgtgcgctgttcgtctgctgatgaccgttcacccgttcgctcgttaa

SEQ ID NO: 40 - DNA, 1065 bp, synthetic construct
  source 1..1065: mol_type = other DNA
atggttcgtaccaccaaaaccagcatggctgctgctagcaccgttgctccggaagttgctatggacgaaggcagcccgagcaccagccaggctcaggttgaactgccgcgtaacctggaagttttcaacgaagcttgcggccacgttttcggcagcagcttcaaccgtgaagacaacagcgttatcagcgacgctgctgctttcctgttcaaaatgcacacccacagcctggacggccaggaagctaaagttctgcgtgctagcgaaaaaaaacgtgaacgtgaaaacgctaaaaaaagccgtaaagctccggaagaaggcatgcgtgttggccgtagcctgatcctgaccagccgttggaccgaatactgcgctacctgcgttccggctctgggcagcaaaatgaaagttatcaaagctagcggcgacgctgctatgatccagatgatgaaagaccacaacagcctgctgcgtgtttgcgttcgtatcgaagtttggaaagctcgttacgttagcctggttgctctggacgaacgtatccagaccctggaagacgctcagtggttcccgtacctgagcggcgacagctaccgtgcttgcccgggcctggttggcggctacttcgctaaaaaagctgctgctggcgaacgtggcaaaaactacaaaaaactgaaccagaccgctatcatcccgccgccgcgtttcctgatcatcggccaccgtctgcagatcggcgaccaggttaccctgcgtgaactgctggctagcatcgcttggggcctgtgcgacggcgttctggctgaatgctggagcccgagccagggcgacggcagcatcggcgttgttgttggcctgccgctgcaggctaccggcagctgcttcctggttgttgctagccacggcctgagcgctatcgctgacagccgtatcgaaggcaccggcaacaccaacctgctggaagaatgcatcgctatccagaaacaggacggcgttatcaaatgcaaacgtagcggcaaaagcctgtaccactgcctgaaagaaaccgctggcgctgttggccgttaa

SEQ ID NO: 41 - DNA, 1032 bp, synthetic construct
  source 1..1032: mol_type = other DNA
atgttcctgctgagccagaccccgatcgctatgcaggttctggttctgaccagctgcctggtttgcgctctggctagcgacgaaagcctgcgtatcaaacgtctgcagagctacctgaacaacacctaccagagccgtgaaatcgaaagcgaaatccgtcgtggcttcgctagcaaattccgtatggaaagctgcagctgcaccatgggcgttcactacatcgttaccccgagcagcggcggcagcttctgcaccggcctgcacgctgttccggacagcttcccggctctgggctacaaactgccgaaagctggcggccgtggcgactggaaagctaccgaagttcgtgttgacgaagacagcggcgttgttctgtacaacgttagccgttgcagccacagcaccgactgccgtgacctggaagtttacagcaccgttctgccgggccagtgcgaatgcacccgtccgaccgttgacgactacaaaaccatgctggctagccgtcagccgaaaagcttcgttgttgctggcctgatcatcctgtgcctgctggctagcagcgttgctatcggcatgggcgtttacaactacgctggcgttatcggcctggctgacgctgctcaggctgacgttagcgaaatctgggaatacctggaagctctgacccgtgaagttaccggcatgaccctgggcgaattctgcagcatcaaaagcctggtttgcaaaagcgacaacatcggcaaattcaaagaacagttcgctgctttcggcgaagctatcctggctatcgttttcggcatgctggaaaaatacaaattcgtttactacctggttctgagcctgatggttctgagcctgctgagcaaactgatcagcctgctgaaacaggttccgttctacggcagcatcaaagttctggttttccgtcgtctgcgtgttgtttgcttcaaaaccttcttcttcatcaaaaaacgtctgaaaaaaaaaggcccgctggaagacgacgaagttccgctgctgccgctgagctaa

SEQ ID NO: 42 - DNA, 954 bp, synthetic construct
  source 1..954: mol_type = other DNA
atgcacttctacctgcaggactgcccgatgagctggctgcgtgttatccgtaccctgaccctgttcagcaccctgttcagccgtagcgaccagtgcgttgacaacatgtggcgtttctacggccgtagcaactacaccagcagcgttgttatcgacggcgacaaatacagcgttgaaggcagctacagcagcagcgactacctggacccggctgttctgaaagttgttctgggcctggacggcagcaacgaagttatcgacagcggcggcagcccgtactaccgttacgacctggaaggcagcaaaggcgaactgcaccacctgaactgcaacttcgttgaaaaacgttgcaacccgaccctgaacttcatgctgggcggcttcgttctgtgcccgggcatcagccgtaaagaactggaaccggttaccgacaaaatcctggaaagccgtggcatcccgggccgtggcaaaatccacaccatcaaaatcagcagcaaactgttcgaaaccagcctgtgcctgagcaaacgtcgtccgatcttcagcacctgcatgctgatgagccgtggcctgtgcaccaactgcaaacgtaccatcgaccgtacctacatgaccagcaacggcttccgtaccgaatacaaatggagctgccgtgacaacagcaccaaacagtgctgggttctggttgaaagcctggaagaaaaccacagcccgtacaaatgccacttcagcgctgttgaagttctgctgccggctgaaatcaaacgtcaccagctgatcagcgaatggagcgctaaacaggacgaagttgcttacaaaaaaagcgacgcttacctgctggctcgtaccttcctgagctacaccaaaatgcgtcgtctgaacccggttatcgacctgagcatcagcccgccggttaccgttcgtagctgctgcaaaatcaacaaatacatgtaa

SEQ ID NO: 43 - DNA, 588 bp, synthetic construct
  source 1..588: mol_type = other DNA
atgagctacaaaatcggcgaactggaacgtatcatcacccgtaaaaacaccctgccgaaagacagcggcagccagaccggcctgttccaccgtctgctgctggaacactacagcggcgctagcaacgtttggttcttctgcgctaccggcttcaccccgaacaccaacggcaccacctggatcgttctgaccagccacccgaccgacggcggcgaaaaagttccgctgaaatggaaatacgaagttagcccgggcctgccggttcgtcgtgttctggctcaggaaggcaccgctgttcgtggcccgaaaggcgcttacctggttaaaggcgacatgcgtctgtgcccgaccaccttctacacccgtcgtgaagctaaatactggctgtgcgctccgagcccgaaattcccgcactggaccaaacgtagcgctctggttaccagcacccgtccgctgaccgaactgagccgtgttgctacctacctggaagctatcagcaaaggcgctaccgacgttaacgaaagctggtgcagctaccaccgtgttggcctggttccgatcccgggcggcatcaccttcgaactgtaa

SEQ ID NO: 44 - DNA, 525 bp, synthetic construct
  source 1..525: mol_type = other DNA
atggctcagatcccgaccctgcgtgaaggccagggcaaactgtacgacttcaccctgaacggcatgaccgttacccgtgacaccgttaacaccgttgttgctctggaattcctggttaacgctagcagcgacctgctgagcctgaccatcggcgaaggcctgagcgaagaaaccaaattcaaacacctgctggttaaacacgctggcatgacccgtaaacgtatcgaagaacgtctgggccgtatcagccgtcgtgttagcgttaccgttgacgctatcatcatcaccaaccgtaaaggccagcgtttcgaattcaaccgtaaacagtacctggacatcgctaaacaggctatgaaactgaaactgccgggcatcaactgcgttgacatcccgaccgctctggctttcctggaagaagttctggctaccgctctgaaagacaccgaaggcagccaggacgaccgtatggctctgaaagctgacaccagcgctgctatcaaccacttccgtgaaatgctgaaataa

SEQ ID NO: 45 - DNA, 351 bp, synthetic construct
  source 1..351: mol_type = other DNA
atgagccgttggatcgaaaacaccgacagcgttagcgttatcctgaccgaaatgagccgtagctacgttaccctgtgccactacccgccgtggagcctgtgctgcggcggcaaaaccagccagccgggccaggtttggatcctggaccgtaaacactgcctggaatgcagcgaaatcagcgaactgatgaccctgggccagccgacctggcaggaaagcgctatggttatcggcagctgggttcaggttgttaccgaacacggcatcctgcacaccagcaccagcgaacgtcaggctccgcgtcgtgttatgtggcgtatcagcgacatggctggcgacaaaaccctgtaa

SEQ ID NO: 46 - DNA, 342 bp, synthetic construct
  source 1..342: mol_type = other DNA
atgagcgttgctgactacctgagcagcgacagcgacagcgaagctgaaagcagcggctgcctggttctgcgtagccgtaaaatcaaaaaaggcaaaaaagctgctagcaaaaaacgtagctggaaaaacgaacgttacggcgctgacgaaagcggcgaagacaacatcgaatggggcgacgaagttgacctggaaatggacgactgcgacagcgctatcccggaatgggctcgtgttgacttcaacccgaaaaaccgtcgtgaccgtgaagacgacggccagagcgacctgagccgtttcagcgaagacttcggcaaaaaaagcctggacgttcagagctaa

SEQ ID NO: 47 - protein, 457 aa, synthetic construct
MSQFEKSFKGRTEVTITEYRSHTVKDVHRSLLTADKSLRKSFCFRNALNQFLDKDLPLLPIRPKLESRVAVKKSKLRSQLSFRPGLTQEEAIDLYNKGYDGDSVSGALQDRVVNEPVAYSSADNDKFHRGLAALGYTLADRALDTCESGFVRAIPTTPCGFICCGPGSFKDSLGFVIKIGEFWHMYDGFQHFVAVEDAKFLASKSPSFWLAKRLAKRLNLVPKEDPSIAAAECPCKKVWEASFARAPTALDPFGGRAFCDQGWVYHRDVGYATANHISQETLFQQALSVRNLGPQGSANVSGSIHTALDRLRAAYSRGTPASRSILQGLANLITPVGENFECDLDRRKLNIKALRSPERYITIEGLVVNLDDVVRGFYLDKAKVTVLSRSRWMGYEDLPQKPPNGTFYCRKRKAMLLISCSPGTYAKKRKVAVQEDRFKDMRVENFREVAENMDLNQ

SEQ ID NO: 48 - protein, 419 aa, synthetic construct
MDSRFAQLTGVFCDDFTYSEGSRRFLSSYSTVERRPGVPVEGDCYDCLKNKWIAFELEGQPRKFPKATVRCILNNDATYVCSEQEYQQICKVQFKDYLEIDGVVKVGHKASYDAELRERLLELPHPKSGPKPHIEWVAPPRLADISKETAELKRQYGFFECSKFLACGEECGLDQEARELILNEYARDREFEFRNGGWIQRYTVASHKPATQKILPLPASAPLARELLMLIARSTTQAGKVLHSDNTSILAVPVMRDSGKHSKRRPTASTHHLVVGLSKPGCEHDFEFDGYRAAVHVMHLDPKQSANIGEQDFVSTREIYKLDMLELPPINKKGDLDRASGLETRWDVILLLECLDSTRVSQAVAQHFNRHRLALSVCKDEFRKGYQLAPEIRGTIPLSSLYYSLCAVRLRMTVHPFAR

SEQ ID NO: 49 - protein, 354 aa, synthetic construct
MVRTTKTSMAAASTVAPEVAMDEGSPSTSQAQVELPRNLEVFNEACGHVFGSSFNREDNSVISDAAAFLFKMHTHSLDGQEAKVLRASEKKRERENAKKSRKAPEAGMRVGRSLILTSRWTEYCATCVPALGSKMKVIKASGDAAMIQMMKDHNSLLRVCVRIEVWKARYVSLVALDERIQTLEDAQWFPYLSGDSYRACPGLVGGYFAKKAAAGERGKNYKKLNQTAIIPPPRFLIIGHRLQIGDQVTLRELLASIAWGLCDGVLAECWSPSQGDGSIGVVVGLPLQATGSCFLVVASHGLSAIADSRIEGTGNTNLLEECIAIQKQDGVIKCKRSGKSLYHCLKETAGAVGR

SEQ ID NO: 50 - protein, 343 aa, synthetic construct
MFLLSQTPIAMQVLVLTSCLVCALASDESLRIKRLQSYLNNTYQSREIESEIRRGFASKFRMESCSCTMGVHYIVTPSSGGSFCTGLHAVPDSFPALGYKLPKAGGRGDWKATEVRVDEDSGVVLYNVSRCSHSTDCRDLEVYSTVLPGQCECTRPTVDDYKTMLASRQPKSFVVAGLIILCLLASSVAIGMGVYNYAGVIGLADAAQADVSEIWEYLEALTREVTGMTLGEFCSIKSLVCKSDNIGKFKEQFAAFGEAILAIVFGMLEKYKFVYYLVLSLMVLSLLSKLISLLKQVPFYGSIKVLVFRRLRVVCFKTFFFIKKRLKKKGPLEDDEVPLLPLS

SEQ ID NO: 51 - protein, 317 aa, synthetic construct
MHFYLQDCPMSWLRVIRTLTLFSTLFSRSDQCVDNMWRFYGRSNYTSSVVIDGDKYSVEGSYSSSDYLDPAVLKVVLGLDGSNEVIDSGGSPYYRYDLEGSKGELHHLNCNFVEKRCNPTLNFMLGGFVLCPGISRKELEPVTDKILESRGIPGRGKIHTIKISSKLFETSLCLSKRRPIFSTCMLMSRGLCTNCKRTIDRTYMTSNGFRTEYKWSCRDNSTKQCWVLVESLEENHSPYKCHFSAVEVLLPAEIKRHQLISEWSAKQDEVAYKKSDAYLLARTFLSYTKMRRLNPVIDLSISPPVTVRSCCKINKYM

SEQ ID NO: 52 - protein, 195 aa, synthetic construct
MSYKIGELERIITRKNTLPKDSGSQTGLFHRLLLEHYSGASNVWFFCATGFTPNTNGTTWIVLTSHPTDGGEKVPLKWKYEVSPGLPVRRVLAQEGTAVRGPKGAYLVKGDMRLCPTTFYTRREAKYWLCAPSPKFPHWTKRSALVTSTRPLTELSRVATYLEAISKGATDVNESWCSYHRVGLVPIPGGITFEL

SEQ ID NO: 53 - protein, 174 aa, synthetic construct
MAQIPTLREGQGKLYDFTLNGMTVTRDTVNTVVALEFLVNASSDLLSLTIGEGLSEETKFKHLLVKHAGMTRKRIEERLGRISRRVSVTVDAIIITNRKGQRFEFNRKQYLDIAKQAMKLKLPGINCVDIPTALAFLEEVLATALKDTEGSQDDRMALKADTSAAINHFREMLK

SEQ ID NO: 54 - protein, 116 aa, synthetic construct
MSRWIENTDSVSVILTEMSRSYVTLCHYPPWSLCCGGKTSQPGQVWILDRKHCLECSEISELMTLGQPTWQESAMVIGSWVQVVTEHGILHTSTSERQAPRRVMWRISDMAGDKTL

SEQ ID NO: 55 - protein, 113 aa, synthetic construct
MSVADYLSSDSDSEAESSGCLVLRSRKIKKGKKAASKKRSWKNERYGADESGEDNIEWGDEVDLEMDDCDSAIPEWARVDFNPKNRRDREDDGQSDLSRFSEDFGKKSLDVQS

SEQ ID NO: 56 - DNA, 32 bp, synthetic construct
  source 1..32: mol_type = other DNA
catggtaccatccgttcggcatccagacgcac

SEQ ID NO: 57 - protein, 33 aa, synthetic construct
GTTTTAGAATCCCCGATCCCGCCGCTGCCACTC

SEQ ID NO: 58 - DNA, 34 bp, synthetic construct
  source 1..34: mol_type = other DNA
gggatcggggattctaaaacggtgttggaatgag

SEQ ID NO: 59 - DNA, 33 bp, synthetic construct
  source 1..33: mol_type = other DNA
catcccgggatcccccatgagctggcgcatctg

SEQ ID NO: 60 - DNA, 38 bp, synthetic construct
  source 1..38: mol_type = other DNA
cattctagagcaaagaccgtcggtggcttcttgtgata

SEQ ID NO: 61 - DNA, 39 bp, synthetic construct
  source 1..39: mol_type = other DNA
catggtacctaaagtgctcagggttttcatgatgctttc

SEQ ID NO: 62 - DNA, 40 bp, synthetic construct
  source 1..40: mol_type = other DNA
catggtaccggacgccccatggagataagcctgctgacac

SEQ ID NO: 63 - DNA, 40 bp, synthetic construct
  source 1..40: mol_type = other DNA
catgagctcctgcaggaaaaactggcgcgatagcatgccg

SEQ ID NO: 64 - DNA, 36 bp, synthetic construct
  source 1..36: mol_type = other DNA
cattctagatagcatacttagttgtaccagcatcat

SEQ ID NO: 65 - DNA, 35 bp, synthetic construct
  source 1..35: mol_type = other DNA
aaagcacacttcgcgcattaccgcgtgcgtggccc

SEQ ID NO: 66 - DNA, 34 bp, synthetic construct
  source 1..34: mol_type = other DNA
taatgcgcgaagtgtgctttccttcgaatgttgc

SEQ ID NO: 67 - DNA, 36 bp, synthetic construct
  source 1..36: mol_type = other DNA
catcccggggcgggtgatatcgatgccgttgagtgt

(19) United States
(12) Patent Application Publication
(10) Pub. No.: US 2026/0034180 A1
(43) Pub. Date: Feb. 5, 2026

(54) COMPOSITIONS, KITS, METHODS, AND METHODS OF ADMINISTRATION RELATING TO EDWARDSIELLA PISCICIDA VACCINE AND/OR ANTIGEN DELIVERY VECTOR SYSTEMS

(71) Applicant: University of Florida Research Foundation, Inc., Gainesville, FL (US)

(72) Inventors: Roy CURTISS, III, Gainesville, FL (US); Banikalyan SWAIN, Gainesville, FL (US)

(21) Appl. No.: 19/100,617

(22) PCT Filed: Aug. 15, 2023
(86) PCT No.: PCT/US2023/072201

§ 371 Date: Feb. 3, 2025

Related U.S. Application Data
(60) Provisional application No. 63/398,514, filed on Aug. 16, 2022.

(51) Int. Cl. A61K 35/74; A61K 39/00; A61K 39/02; A61K 39/12; A61P 31/04; A61P 31/14; C12N 15/74

(52) U.S. Cl. CPC A61K 35/74; A61K 39/0208; A61K 39/025; A61K 39/12; A61P 31/04; A61P 31/14; C12N 15/74; A61K 2039/522; A61K 2039/523; A61K 2039/53; A61K 2039/542; A61K 2039/552; A61K 2039/575

(57) ABSTRACT

In one aspect, the disclosure relates to recombinant bacterial vectors including a gene encoding at least one antigen from Aeromonas hydrophila or tilapia lake virus, methods of making the same, vaccines incorporating the same, and methods of inducing an immune response in the subject and/or preventing infection by a pathogen in the subject using the same. In one aspect, the subject is a fish in an aquaculture system. In an aspect, the vector or vaccine can be administered by bath immersion or intracoelomic injection and, in some cases, can confer protection against an additional pathogen such as, for example, Edwardsiella piscicida. In any of these aspects, the vectors are susceptible to antibiotics and do not persist in the environment.

### CROSS-REFERENCE TO RELATED APPLICATIONS

This application claims the benefit of U.S. Provisional Application No. 63/398,514, filed on Aug. 16, 2022, which is incorporated herein by reference in its entirety.

### STATEMENT REGARDING FEDERALLY SPONSORED RESEARCH OR DEVELOPMENT

This invention was made with government support under Grant No. 2018-67015-28286, awarded by the United States Department of Agriculture, National Institutes of Food and Agriculture. The government has certain rights in the invention.

### CROSS-REFERENCE TO SEQUENCE LISTING

This application contains a sequence listing filed in ST.26 format entitled “222111-2990_FINAL_Sequence_Listing.xml” created on Aug. 15, 2023, and having a file size of 77,349 bytes. The content of the sequence listing is incorporated herein in its entirety.

### BACKGROUND

Aquaculture is becoming a most important segment of the farm economy to respond to an expanding need to produce high-quality animal protein at a reasonable cost to feed an increasing human population. Aquaculture is an approximately $1.2 billion industry, and catfish are one of the dominant aquaculture species in the United States. Catfish growers in the U.S. had sales of 379 million dollars during 2019, up 5 percent from 360 million dollars the previous year. Disease outbreaks are among the primary limiting factors in catfish production. Infectious diseases account for the most significant percentage of losses, with around 65% of the fry and fingerlings lost during production. There is a trend toward the increased incidence and prevalence of Edwardsiella and Aeromonas spp. infection in US catfish aquaculture particularly in channel catfish (Ictalurus punctatus). Tilapia farming is also increasing in importance since tilapia are one of the most efficient converters of provided food to fish weight such the tilapia now ranks second in the world in tonnage produced with production in the US expanding by use of indoor insulated systems in northern and Midwestern states and pond systems in warmer Southeastern states. Tilapia lake virus (TiLV), also known as Tilapia tilapinevirus, is one of the most significant infectious agents causing relatively high mortality and economic losses for tilapia farmers. The mortality rate in natural TiLV outbreaks ranges from 20% to 90%, while cumulative mortalities from experimental infection range from 66% to 100%. Tilapia lake virus (TiLV) was first detected in the United States in March 2019 and it poses a serious threat to U.S. agriculture. Currently, no antiviral therapy or vaccines are available for the control of this disease. Thus, there is an urgent requirement for the researchers to understand the current emergence of this viral disease and extensive involvement of scientific work is required for the development of effective vaccine against this deadly virus. Edwardsiella is a gram-negative early progenitor of the Enterobacteriaceae that eventually gave rise to Escherichia and Salmonella. It is a facultative intracellular pathogen that shares many properties with Salmonella. The genus Aeromonas currently comprises 31 species. Among the motile aeromonads, A. hydrophila is a gram-negative, facultative, anaerobic rod that is predominant in aquatic environments. It is the main etiological agent of motile aeromonad septicemia (MAS). Treatment of infections caused by bacterial pathogens with antibiotics also has a negative impact in selecting for antibiotic-resistant bacterial pathogens, some of which could be passed through the food chain to humans and cause outbreaks of Aeromonas hydrophila food poisoning. Live recombinant immersion vaccines, which protect against several diseases by expressing multiple protective antigens at low cost, have not yet been developed for the aquaculture industry. These efforts are therefore directed at addressing this need for safe efficacious vaccines that would be cost-effective to manufacture and safe to administer. Edwardsiella is a gram-negative, facultative intracellular pathogen that shares many properties with Salmonella. Many species of Edwardsiella are invasive pathogens of fish such as E. ictaluri that causes enteric septicemia in catfish and E. piscicida that has a broader host range and causes invasive disease in many commercially important fresh-water and marine fish including catfish, tilapia, trout, mullet, salmon, carp and striped bass. Factors that have contributed to the severity of Edwardsiella infections in fish include intensive fish farming methods, development of multi-antibiotic resistance due to the overuse of antimicrobial chemicals in aquaculture and agriculture and the bacterium's broad host range. For the treatment and prevention of these infections, the use of biological control methods such as vaccination needs to be developed. The most used current method for vaccination in the aquaculture industry is by intracoelomic (i.e.) injection. This method of immunization is expensive, as stated above. Thus, injectable vaccines are seldom used in the aquaculture industry, but bath live, attenuated bacterial vaccines are commercially used but not yet very effective or safe.

A. hydrophila is the predominant etiological agent of motile Aeromonas septicemia (MAS), a dangerous bacterial disease of freshwater fish. The acute form of the disease is characterized by rapid fatal septicemia, and the most significant signs are exophthalmia, reddening of the skin, and accumulation of fluid in the scale pockets. Even when tissue damage in the liver and kidneys is extensive, the heart and spleen are not necessarily damaged. However, splenic ellipsoids are often centers of intense phagocytic activity by macrophages. Impact of epidemic A. hydrophila on catfish aquaculture: In 2009, epidemic A. hydrophila outbreaks occurred in at least 48 catfish farms in West Alabama causing an estimated loss of more than 3 million pounds of market size channel catfish. In 2010, the disease reemerged and spread to at least 60 farms (including the 48 affected in 2009) with an estimated loss of 2,400,000 lbs. of catfish. 2011 and 2012 were also similar to 2010, with a loss of over 2 million lbs. of catfish per year. Up to 2014, these epidemic outbreaks of MAS were responsible for an estimated loss of more than $12 million in catfish aquaculture operations in the southeastern U.S.

Recently, there has been growing interest in the use of live attenuated vaccines (LAVs) against bacterial fish pathogens. This interest can be attributed to the superior protection afforded by live vaccines for several animal species. In general, live vaccines stimulate a more robust immune response than bacterins do. In constructing an effective live attenuated vaccine, the goal is to alter the microbe's genome in such a way that its pathogenicity is minimized but it is still able to induce an immune response. Traditional in-frame gene deletion can result in over-attenuation of the bacterial strain, impeding adequate host tissue colonization and increasing susceptibility to host defenses.

At present, no commercial vaccine against A. hydrophila is available. Several studies have proved that injection or immersion vaccination with heat- or formalin-inactivated bacterins may provide protection. A LAV trial has been conducted by using antibiotic-resistant epidemic A. hydrophila. Although this attenuated epidemic A. hydrophila strain showed efficacy in protecting fish against MAS in laboratory challenges, it has not been effective in preventing the outbreaks in pond trials. It is also resistant to antibiotics and this is an undesirable attribute for live vaccines to be introduced into the environment. Therefore, there is an urgent need for a practical, genetically defined, safe, and efficacious A. hydrophila vaccine that is sensitive to all antibiotics to which gram-negative bacteria are sensitive.

To contend with viral pathogens that often possess surface proteins necessary for infection that have been post-translationally modified, frequently by glycosylation, that can occur in eukaryotic cells but not in bacteria, successful vaccination against such viral pathogens requires that genes encoding for these viral proteins be introduced into cells of the vaccinated eukaryotic animal host. For this reason, DNA vaccines were first described to transfer by injection such viral genes to induce protection against influenza virus infections. Later attempts were made to deliver DNA vaccines to animal hosts by genetically modified Shigella and Salmonella but success was very limited for multiple reasons. Early DNA vaccines were derived from bacterial plasmids to enable their amplification and production in Escherichia coli strains. However, bacterial plasmids have no experience in eukaryotic cells and are therefore subject to degradation by host nucleases and have no means to migrate to the nucleus necessary for transcription and translation of encoded genes specifying protective antigens. In addition, the bacteria used generally had to be lysed by the vaccinated animal host and also as one of their virulence attributes induced pyroptosis/apoptosis leading to degradation of the nuclear machinery necessary for transcription and translation of the DNA vaccine encoded protective antigens. Furthermore, when strains of bacteria were used that underwent lysis to release the DNA vaccine construct, lysis was often too rapid before the bacteria invaded into host cells and also released bacterial endonucleases that destroyed the integrity of the DNA vaccines composed or circular DNA. A successful DNA vaccine approach, if devised, would also likely lead to the development of other DNA vaccines for use in aquaculture, such as, for example, a vaccine to protect tilapia against infection with TiLV, which is currently a significant source of mortality and disease.

Despite advances in disease prevention research in the field of aquaculture, there is still a scarcity of vaccines and methods for developing the same that are potent, safe and efficacious against bacterial and viral pathogens in commercially valuable fishery stocks. These needs and other needs are satisfied by the present disclosure.

### SUMMARY

In accordance with the purpose(s) of the present disclosure, as embodied and broadly described herein, the disclosure, in one aspect, relates to recombinant bacterial vectors including a gene encoding at least one antigen from Aeromonas hydrophila or tilapia lake virus, methods of making the same, vaccines incorporating the same, and methods of inducing an immune response in the subject and/or preventing infection by a pathogen in the subject using the same. In one aspect, the subject is a fish in an aquaculture system. In an aspect, the vector or vaccine can be administered by bath immersion or intracoelomic injection and, in some cases, can confer protection against an additional pathogen such as, for example, Edwardsiella piscicida. In any of these aspects, the vectors are susceptible to antibiotics and most importantly display biological containment so as not to persist in the environment.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show evolutionary relationships and structural similarity of E. piscicida MurA with other bacteria. (FIG. 1A) Amino acid sequences of MurA of various bacteria were retrieved from the GenBank database and the unrooted phylogenetic tree of MurA was inferred using the Neighbor-Joining method. The percentage of replicate trees in which the associated taxa clustered together in the bootstrap test (500 replicates) are shown next to the branches. Evolutionary analyses were conducted in MEGA X. (FIG. 1B) 3D structure of MurA protein of E. piscicida, E. ictaluri, and S. typhimurium were predicted by using (PS)2: Protein Structure Prediction Server Version 3.0 web portal.

FIG. 2A shows a diagram of chromosomal deletion-insertion mutation of ΔP_{murA}: substitution of a tightly regulated araC P_{araBAD} cassette for the wild-type promoter of E. piscicida murA gene resulting in arabinose-regulated murA expression. FIG. 2B shows the wild-type murA and ΔP_{murA} promoter region sequence and a deletion of the promoter for the murA gene (57 bp) with replacement by the araC P_{araBAD} cassette. A modified Shine-Dalgarno sequence is represented in red. FIG. 2C shows genotype verification of ΔP_{murA} (χ16016). FIG. 2D shows a schematic illustration of arabinose regulation of chromosomal expression of the murA gene. FIG. 2E shows the mechanisms of E. piscicida lysis systems and a schematic representation of arabinose-dependent ΔP_{murA} (χ16016) cell wall synthesis. FIG. 2F shows the model for the bacterial call wall peptidoglycan biosynthesis pathway in which MurA catalyzes the first step in the peptidoglycan biosynthesis pathway. FIG. 2G shows phenotypic characterization of ΔP_{murA180}::TT araC P_{araBAD} murA (χ16016) in the presence or absence of arabinose on LB agar plates. FIG. 2H shows growth curves for J118 and χ16016 in presence or absence of arabinose.

FIGS. 3A-3C show abilities of E. piscicida wild-type and ΔP_{murA} (χ16016) strains to attach to, internalize into and replicate in a fish cell line. (FIG. 3A) Attachment assay: Endothelial progenitor cells (EPC) cells were infected by J118 or χ16016 in presence or absence of arabinose at a multiplicity of infection (MOI) of 10 and incubated for 1 h to reach an adequate level of infection. The infected EPC cells were washed three times, lysed with 1% (v/v) Triton X-100 in PBS and lysates were platted on LB agar plates with or without arabinose to assay the attached bacterial cells. (FIG. 3B) Internalization assay: After infection, the cells were incubated for another 1 h in cell culture medium containing gentamicin (100 mg/mL). Then, cells were lysed with 1% Triton X-100 in PBS, and lysates were plated on LB agar with or without arabinose plates to count the internalized bacterial cells. (FIG. 3C) Replication assay: After gentamicin treatment, cells were incubated with growth media, and the intracellular bacterial population was assayed two and four hours later. Cells were washed three times with PBS and lysed with 0.1 mL of 1% (vol/vol) Triton X-100 solution and the number of bacteria was assayed by platting lysates on LB agar with or without arabinose plate.

FIGS. 4A-4F show activation of TLR4 and TLR5, TLR8, TLR9, NOD1 and NOD2 by E. piscicida ΔP_{murA} (χ16016) strain. HEK-Blue™ mTLR4, mTLR5, mTLR8, mTLR9, mNOD1 and mNOD2 were treated with χ16016 or wild-type strain J118 with an MOI of 1. After 3, 6, 12 and 24 h of incubation, secreted embryonic alkaline phosphatase (SEAP) activity was determined at 655 nm according to the manufacturer's recommendations (Invivogen). All samples were measured in triplicate. The toll-like receptors (TLRs) and nucleotide-binding and oligomerization domain (NODs) receptor stimulation was expressed relative to the level of SEAP activity of untreated control cells. Differences between uninfected (control) and infected cell groups were analyzed by two-way ANOVA, where asterisks (*) indicate a significant difference (*P<0.05, **P<0.01, ***P<0.001, ****P<0.0001), with respect to the control cells.

FIGS. 5A-5C show colonization and lysis of χ16016 in catfish tissues. (FIG. 5A) Catfish were infected with χ16016 or J118 by bath immersion. Kidney and intestine were collected from fish at day 1, 2, 3 and 4 of post-vaccination (five fish in each group at each time point). The tissues were homogenized in 200 μL of buffered saline with gelatin (BSG) and plated on LB agar plates containing colistin sulfate (Col) (12.5 pg/mL) and 0.2% arabinose. The plates were incubated at 30° C. for 48 h and the colonies were counted. The colonization data was a combination of three independent assays. (FIG. 5B) For the lysis study, catfish were immunized with χ16016 by i.e. injection. Kidney was collected from fish at every alternate day up to 16 days of post-vaccination (five fish at each time point). The tissues were homogenized in 200 μL of BSG and plated on LB agar plates containing colistin sulfate and arabinose. The plates were incubated at 30° C. for 48 h and the colonies were counted. The data was a combination of three independent assays.

FIG. 6 shows a summary diagram of the experimental setup sampling of catfish. Catfish fingerlings were divided into immunized (χ16016) and control (PBS) groups, and each group was split into triplicate tanks (n=30 fish per tank). Fish were immunized with either PBS (control) or χ16016 (with dose 5×10⁶ CFU/mL) by bath immersion and marked as day 0. Different tissue samples i.e., gill, kidney, intestine, spleen and blood were collected at 3, 5 and 7 days of post vaccination to measure the immune response by qRT-PCR. On day 14, fish were booster immunized with either PBS (control) or χ16016 with approximately same initial vaccination dose by bath immersion. On day 28, Serum and skin mucus were collected (five fish from each group) to quantify the IgM by ELISA and fish were (both control and immunized) i.e. challenged with 3×10⁵ CFU of J118/fish. Fish were monitored up to 21 days after challenge and mortality levels were recorded. Serum and skin mucus were collected (five fish from each group) on day 49 to quantify the IgM and all the survived fish were humanely euthanized.

FIGS. 7A-7E show changes in the expression patterns of il-8, i1-1β, tnf-a, il-6 and ifn-γ genes in χ16016 vaccinated and control catfish. Catfish were vaccinated with χ16016 by bath immersion. At 3, 5 and 7 days of post-immunization, total RNA was extracted from the gills, kidney, intestine, spleen and blood and cDNA was prepared. The qRT-PCR assay was conducted to analyze the expression of the il-8, il-1β, tnf-α, il-6 and ifn-γ genes using 18S rRNA as an internal control. The results are expressed as mean±standard error (bars) from three separate experiments. Differences between uninfected (control) and infected groups were analyzed by two-way ANOVA, where asterisks (*) indicate significant difference (*P<0.05, **P<0.01, ***P<0.001, ****P<0.0001) with respect to the control group.

FIGS. 8A-8E show changes in the expression patterns of the T and B cells-related genes in χ16016 vaccinated and control catfish. Catfish were vaccinated with χ16016 by bath immersion. At 3, 5 and 7 days of post-immunization, total RNA was extracted from the gills, kidney, intestine, spleen and blood and cDNA was prepared. The qRT-PCR assay was conducted to analyze the expression of cd4-1, cd4-2, cd8-α, cd8-β and mhc-ii genes using 18S rRNA as an internal control. The results are expressed as mean±standard error (bars) from three separate experiments. Differences between uninfected (control) and infected groups were analyzed by two-way ANOVA, where asterisks (*) indicate significant difference (*P<0.05, **P<0.01, ***P<0.001, ****P<0.0001) with respect to the control group.

FIG. 9A shows analysis of anti-LPS antibody responses in control and immunized catfish: Serum and mucosal immunoglobulin M (IgM) responses to E. piscicida lipopolysaccharide (LPS) were measured by ELISA at 4 weeks (4 w) post-vaccination with χ16016 and 3 weeks after J118 challenge (7 w) of immunized fish. The results are expressed as mean±standard error (bars) from three separate experiments. Differences between treated and control groups were analyzed by two-way ANOVA, where asterisks (*) indicate significant difference (*P<0.05). FIG. 9B shows survival rate of vaccinated fish after wild-type E. piscicida challenge: Control and 4 weeks post-vaccinated catfish were i.e. challenged with 3×10⁵ CFU/dose of wild-type E. piscicida (J118). Mortality was recorded daily and represented as percent survival. FIG. 9C shows the clinical observation of catfish after challenge with E. piscicida.

FIGS. 10A-10C show a regulated lysis vector with T3SS antigen delivery pG8R110 with p15A on (FIG. 10A) and improved T2SS bla SS for antigen delivery pG8R114 with pBR ori (FIG. 10B).

FIG. 11 shows a western blot illustration depicting isopropyl β-D-1-thiogalactopyranoside (IPTG)-dependent synthesis of eight protective A. hydrophila antigens encoded on pG8R114 in E. coli χ6212(pYA232). Antigen synthesis was analyzed in IPTG induced (1) and uninduced (U) cells by western blotting with anti-His-tag antibody.

FIG. 12 shows a western blot illustration depicting arabinose-dependent synthesis of seven protective A. hydrophila antigens encoded on pG8R114 in E. piscicida χ16035. Antigen synthesis was analyzed in arabinose presence (Ara+) and absence (Ara−) cells by western blotting with anti-His-tag antibody.

FIG. 13A shows a schematic representation of secondary structure of A. hydrophila antigen AOKFG8, which was predicted by the SMART program. FIG. 13B shows the 3D structure of A. hydrophila AOKFG8 antigen and its different domains (Plug, TonB, and OMP) were predicted by using Phyre2 web portal.

FIGS. 14A-14B show SDS PAGE (FIG. 14A) and western blot (FIG. 14B) analyses depicting IPTG-dependent synthesis of different domains of A. hydrophila protective antigen AOKFG8 encoded on pG8R114 in the E. coli cells χ6212(pYA232). Antigen synthesis was analyzed in IPTG induced (i) and uninduced (u) cells by western blotting with anti-His-tag antibody.

FIG. 15A shows a western blot illustration depicting arabinose-regulated synthesis of TonB and OMP domains of A. hydrophila protective antigen AOKFG8 encoded on pG8R114 in RAEV cells χ16035. Antigen synthesis was analyzed in arabinose presence (+) and absence (−) cells by western blotting with anti-His-tag antibody. FIG. 15B shows a western blot illustration depicting synthesis of TonB (pG8R8517) domains of A. hydrophila protective antigen AOKFG8 encoded on pG8R114 in RAEV cells χ16018. Antigen synthesis was analyzed by western blotting with anti-His-tag antibody.

FIG. 16A shows a plasmid map of pG8R8517: A. hydrophila tonB gene segment was introduced into pG8R114; lysis vectors with improved T2SS bla SS pBR ori. FIG. 16B shows in vitro demonstration of programmed lysis of E. piscicida strain χ16018(pG8R8517) in the presence or absence of arabinose.

FIG. 17A shows induction of immune protection by RAEV-Ah strain χ16018(pG8R8517) against virulent wild-type A. hydrophila in zebrafish. FIG. 17B shows A. hydrophila infected zebrafish showing typical aeromoniasis disease symptoms.

FIG. 18 shows growth curves of mutant E. piscicida strains χ16017 and χ16017(pG8R114) with and without DAP/arabinose.

FIG. 19 shows LacI synthesis in mutant E. piscicida strain χ16032 carrying the ΔrelA20::araC P_{araBAD} lacI TT deletion-insertion mutation, in presence and absence of arabinose, detected by western blotting using antiserum against the Lac repressor (LacI).

FIG. 20 shows colonies of wild-type E. piscicida (J118) and ΔendA11(χ16029) strains stained by methyl green after toluene incubation. Wild-type (WT) colonies uptake little or no stain, while ΔendA11(χ16029) colonies are stained.

FIG. 21 shows PCR amplification of 10 different tilapia lake virus (TiLV) open reading frame (ORF) segments.

FIGS. 22A-22B show plasmids pCHC104 and pCHC107 that were individually inserted into χ16035. Colonies were grown in LB broth with or without arabinose for overnight at 30° C. Western blots were performed with anti-His horseradish peroxidase (HRP) (R&D Systems cat #MAB050H) and developer reagent Pierce 1-Step Ultra TMB blotting solution (Thermo Fisher cat #37574). Genotype of χ16035: ΔasdA10 ΔP_{fur170}::TT araC P_{araBAD} fur ΔP_{crp}::araC P_{araBAD} crp TT ΔrelA::araC P_{araBAD} lacI TT. Plasmids pCHC104 and pCHC107 have parent vector pG8R114, with inserts of segment 6 and segment 5Δ, respectively, and encoding protein sizes of 38.5 kDa and 21.9 kDa, respectively.

FIG. 23 shows analysis of TiLV antigen synthesis in E. coli BL-21. pET28(a)⁺ vectors encoding the TiLV gene segments were electroporated into BL-21 individually. Cells were induced by 1 mM IPTG for 4 hrs (I: induced, U: uninduced) and protein synthesis was confirmed by western blotting with anti-His-tag antibody. Lanes are identified as follows 20: BL-21(pG8R9020), 21: BL-21(pG8R9021), 24: BL-21(pG8R9024), 25: BL-21(pG8R9025), 26: BL-21(pG8R9026), 27: BL-21(pG8R9027), 28: BL-21(pG8R9028), 22: BL-21(pG8R9022), 23: BL-21(pG8R9023); see also Table 10.

FIGS. 24A-24B show TiLV antigen synthesis by vector pcDNA3.1(+)/MYC-HIS/A in an HEK293T cell line. TiLV gene segments were inserted into pcDNA/3.1/A vector. HEK293T cells were transfected with pcDNA/3.1/A-TiLV plasmids using Lipofectamine 2000 and cells were incubated at 37° C. with 5% CO₂. After 24 and 48 hours of transfection, cells were harvested and lysed in sample loading buffer. Cell lysates were separated using 12% sodium dodecyl sulfate-polyacrylamide gel electrophoresis (SDS-PAGE) and transferred onto a nitrocellulose membrane for western blot analyses. Anti-His-tag antibody was used for the detection of the TiLV antigen synthesis. SuperSignal™ West Pico PLUS Chemiluminescent Substrate was used to develop the blot. Cells transfected with pG8R9029, pG8R9030, and pG8R9031 (FIG. 24A) and pG8R9035 (FIG. 24B) showed positive signals. See also Table 11.

FIG. 25A shows a map of DNA vaccine vector pYA4545. Plasmid sequences include the rrfG, trpA, and 5S ribosomal RNA transcriptional terminators, the P_{araBAD}, P22 P_{R}, and P_{CMV} promoters, the araC gene, and start codon-modified murA and asdA genes, DTS (I), DTS (II), and SV40 late polyA. FIG. 25B shows a demonstration of enhanced green fluorescent protein (EGFP) synthesis by DNA vector pYA4545 in a fish cell line (EPC cells).

FIGS. 26A-26B show IAG52B i-antigen synthesis by DNA vector pYA4545 in fish cell line (EPC), confirmed by western blotting. FIG. 26C shows an in vitro demonstration of programmed lysis of E. piscicida strain χ16027(pYA4545) and χ16027(pG8R8041) in the presence or absence of arabinose. FIG. 26D shows a vector map of pG8R8041 with the IAG52B antigen gene cloned into the DNA vaccine vector pYA4545.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a new mucosal antigen delivery system for aquaculture that prevents important infectious diseases and therefore increase the sustainability and profitability of the finfish aquaculture industry in the US. Channel catfish (Ictalurus punctatus) is the top species produced in US aquaculture and motile Aeromonas septicemia (MAS), caused by virulent Aeromonas hydrophila (vAh), is one of the most severe diseases that causes mass mortality of farmed catfish. Since 2009 outbreak of MAS in farmed catfish in the southeastern United States, recurring outbreaks of MAS have resulted in the loss of millions of pounds of food-size fish annually. In order to prevent and control the outbreak, antibiotics are widely used in aquaculture, resulted in the emergence of bacterial resistance. Although vaccination is an effective preventive method, there is no commercial vaccine available against MAS. Thus, there is an urgent requirement for the development of effective vaccine against this deadly disease. In this regard, disclosed herein is a mucosal delivered recombinant attenuated Edwardsiella vaccine (RAEV) vector system encodes A. hydrophila protective antigen(s) (RAEV-Ah) to protect catfish against MAS. The disclosed system is a recombinant attenuated Edwardsiella vaccine (RAEV) vector system that is sensitive to all antibiotics, with regulated delayed attenuation, regulated-delayed antigen synthesis and regulated delayed lysis attributes that exhibits biological containment so as not to persist for more than a few weeks in vaccinated fish and is unable to survive in nature. This last feature is important for vaccines to be administered in aqueous environments. In one aspect, the regulated delayed attenuation and regulated delayed lysis features designed into these E. piscicida strains enable them to efficiently colonize host lymphoid tissues and allow release of the bacterial cell contents after lysis. In another aspect, none of the bacterial vaccine cells are able to survive and thus exhibit complete biological containment. In a further aspect, these RAEV vector systems thus have the same well-documented safety and efficacy attributes of known systems using Salmonella vectors. In a still further aspect, these vaccine vector strains efficiently synthesize and deliver protective antigens from bacterial, viral and parasitic pathogens of fish to induce protective immunity after mucosal bath (needle-free) immersion vaccination of fish. In one aspect, these genetically reprogrammed vaccine constructs have solved the problem inherent in the Pasteur approach of generating live bacterial and viral vaccines in which introducing attenuating alterations leads to a concomitant reduction in immunogenicity compared to infection with the wild-type parental pathogen. Thus, the disclosed RAEV and RASV constructs with the regulated-delayed lysis in vivo attributes induce maximal mucosal, systemic and cellular immune responses against pathogens whose protective antigens are delivered by the vaccine construct. In another aspect, the immunogenicity and protective efficacy of one of the RAEV vaccine strains χ16035 (ΔasdA10ΔP_{fur170}::TT araC P_{araBAD} fur ΔP_{crp68}::TT araC P_{araBAD} crp ΔrelA20::araC P_{araBAD} lacI TT) encoding an A. hydrophila antigen (RAEV-Ah) has been analyzed. In a further aspect, the A. hydrophila antigen gene was incorporated into the plasmid pG8R114, which has the regulated delayed lysis in vivo phenotype and employed the balanced-lethal vector-host concept for stable plasmid maintenance to ensure that live RAEV strains are sensitive to all antibiotics and thus unable to disseminate antibiotic resistance when RAEVs are used in non-enclosed environments. The disclosed vaccine construct, as fully described herein, showed 60% protection against virulent A. hydrophila (ML-10-51K, LD₅₀=7×10²) 20×LD₅₀ challenge compared to the unimmunized group in zebrafish. Similar or better protective efficacy of the same construct in catfish is predicted.

In another aspect, the disclosed vaccines and methods make use of a regulated-delayed-attenuation system since that phenotypic expression is dependent upon the availability of some externally supplied nutrient. In one aspect, replacing the fur and crp gene promoters in E. piscicida with a highly regulated araC P_{araBAD} cassette makes it so that expression is dependent upon the presence of arabinose. In a further aspect, following vaccination, the strain appears to elicit the same type of virulence expected from the wild type, but the absence of arabinose within host tissue in vaccinated fish allows for gradual attenuation and the prevention of full-blown infection.

The regulated delayed lysis system, relies on plasmid vectors with regulated expression of the asdA and murA genes encoding enzymes required for synthesis of DAP and muramic acid respectively, both of which are essential components of the peptidoglycan layer of the bacterial cell wall. Since the product of the murA gene is phosphorylated, and thus cannot be taken up by bacteria, murA deletions are lethal. It is therefore necessary to create a conditional-lethal murA mutation by replacing the chromosomal murA promoter with the araC P_{araBAD} activator-promoter. The ΔP_{murA180}::TT araC P_{araBAD} murA mutation was introduced into wild-type E. piscicida J118 to yield χ16016, which only grew in broth containing arabinose. When χ16016 was deprived of arabinose, cells lysed.

### Recombinant Bacterial Vectors

In one aspect, disclosed herein is a recombinant, attenuated, biologically-contained Edwardsiella bacterial vector displaying regulated delayed lysis attributes, the vector including a gene encoding at least one antigen from a fish pathogen; wherein the at least one antigen or a DNA vaccine vector containing the gene encoding the at least one antigen induces expression of host genes for enhancement of innate immunity after entry of the antigen or DNA vaccine vector into a eukaryotic host cell. In an aspect, the vector can be or include an E. piscicida strain such as, for example, one derived from E. piscicida J118.

In one aspect, the pathogen can be a bacterial pathogen, a viral pathogen, or a parasitic pathogen. In a further aspect, the bacterial pathogen can be Aeromonas hydrophila. In an alternative aspect, the viral pathogen can be tilapia lake virus (TiLV).

In another aspect, the at least one antigen is selected from outer membrane proteins and their domains including, but not limited to, CC002501, AGM43135, AGM42919, AOKFG8, AOKGW8, AOKQ46, AOKQZ1, AOKIU8, AGH12866, a TonB domain of AOKFG8, an OMP domain of AOKFG8, a Plug domain of AOKFG8, or any combination thereof.

In still another aspect, the at least one antigen is a TiLV antigen and the gene encoding the at least one antigen has at least 80%, at least 85%, at least 90%, at least 95%, at least 99%, or at least 99.9% sequence identity with any one of SEQ ID NOs. 47-55.

In any of these aspects, entry of the antigen or a vector encoding the same into a host cell induces expression of at least one cytokine gene such as, for example, a gene for IL-8, IL-1β, TNF-α, IL-6, INF-γ, or any combination thereof. In still another aspect, the cytokine can be produced in organs including, but not limited to, gills, kidneys, intestine, spleen, blood, or any combination thereof. In another aspect, entry of the antigen or vector encoding the same into a eukaryotic cell causes expression of genes encoding CD4-1, CD4-2, CD8-α, CD8-β, MHC-II, or any combination thereof.

In another aspect, the gene encoding the at least one antigen is incorporated into a plasmid such as, for example, pG8R111, pG8R114, or pYA4545. In one aspect, DNA vaccine vector pYA4545, which has the potential of plasmid nuclear import and resistance to attack by host nucleases, complements the chromosomal asdA and murA imposed requirements of the disclosed RAEV regulated delayed lysis strains. Plasmid sequences include the rrfG, trpA, and 5S ribosomal RNA transcriptional terminators, the P_{BAD}, P22 P_{R}, and P_{CMV} promoters, the araC gene, and start codon-modified murA and asdA genes, DTS (I), DTS (II), and SV40 late polyA.

In one aspect, the recombinant bacterial vector transiently colonizes at least one tissue type in the subject, wherein colonization by the vector induces release of the at least one antigen.

In some aspects, the at least one tissue type can be internal lymphoid tissues. In another aspect, transient colonization can allow for production of antigens without persistence and full-blown infection by the pathogen or E. piscicida in the subject, thereby providing a delayed attenuation effect. In a further aspect, this delayed attenuation effect or phenotype can prevent release of active pathogen into the environment surrounding the subject.

In one aspect, in the recombinant bacterial vector, at least one gene promoter has been replaced with an araC P_{araBAD} cassette to create the delayed attenuation phenotype. In another aspect, the at least one gene promoter can be for the fur gene, the crp gene, or both the fur and crp genes. In any of these aspects, presence of the araC P_{araBAD} cassette causes vector dependence on arabinose. In a further aspect, vector dependence on arabinose prevents full infection by E. piscicida with induction of disease symptoms. In yet another aspect, the vector does not persist in vivo after administration to the subject, or persists for about two weeks or less in vivo after administration to the subject.

In one aspect, the vector is sensitive to one or more antibiotics used to treat infections caused by Edwardsiella bacteria.

### Vaccines

In one aspect, the disclosed vaccine displays biological containment as a dual property conferred by both chromosomally inserted mutations and plasmid and DNA vaccine vector encoded sequences that result in a regulated delayed lysis in vivo due to the inability for sustained synthesis of unique essential constituents of the peptidoglycan layer of the cell wall. Lysis results since the rigidity and integrity of the call is dependent on this peptidoglycan layer.

In another aspect, disclosed herein is a vaccine that includes the recombinant bacterial vector as described herein. In another aspect, the vaccine further includes at least one adjuvant, such as, for example, a component derived from lysed E. piscicida cells. In another aspect, the at least one component can be selected from lipopolysaccharide (LPS), flagella, Edwardsiella CpG DNA, lipoprotein, a peptidoglycan fragment, or any combination thereof. In a further aspect, the peptidoglycan fragment can be diaminopimelic acid, muramyl dipeptide, or any combination thereof.

### Method for Generating an Immune Response

In one aspect, disclosed herein is a method for generating an immune response to at least one pathogen in a subject, the method including at least the step of administering a disclosed recombinant bacterial vector or vaccine to the subject. In some aspects, the subject is a fish in an aquaculture system. In a further aspect, the subject can be a fry, juvenile, adult, or spawning fish selected from a species including, but not limited to, tilapia, catfish, trout, mullet, salmon, carp, or striped bass.

In one aspect, the virus is TiLV. In another aspect, the bacterial pathogen is A. hydrophila. In some aspects, the vaccine can additionally generate an immune response against at least one additional pathogen. In a further aspect, the at least one additional pathogen can be wild type Edwardsiella piscicida, Edwardsiella ictaluri, Edwardsiella hoshinae, Edwardsiella tarda, or any combination thereof.

In some aspects, administration is carried out by immersion of the subject in a bath containing the vector or vaccine. In a further aspect, administration includes introducing up to 4×10⁸ CFU of recombinant bacterial vector per mL of the bath. In some aspects, the vaccine can be administered to multiple subjects simultaneously by immersing all of the subjects in the bath at the same time. In other aspects, the vaccine can be administered by intracoelomic (i.e.) injection of the subject.

In an aspect, administration can be conducted once, or can be conducted twice, wherein a booster administration occurs about six weeks after an initial administration.

### Method for Preventing Infection by a Pathogen

In another aspect, disclosed herein is a method for preventing infection by a pathogen in at least one subject, the method including at least the step of administering a disclosed recombinant bacterial vector or vaccine to the subject. In some aspects, the subject is a fish in an aquaculture system. In a further aspect, the subject can be a fry, juvenile, adult, or spawning fish selected from a species including, but not limited to, tilapia, catfish, trout, mullet, salmon, carp, or striped bass.

In one aspect, the virus is TiLV. In another aspect, the bacterial pathogen is A. hydrophila. In some aspects, the vaccine can additionally generate an immune response against at least one additional pathogen. In a further aspect, the at least one additional pathogen can be wild type Edwardsiella piscicida, Edwardsiella ictaluri, Edwardsiella hoshinae, Edwardsiella tarda, or any combination thereof.

In some aspects, administration is carried out by immersion of the subject in a bath containing the vector or vaccine. In a further aspect, administration includes introducing up to 4×10⁸ CFU of recombinant bacterial vector per mL of the bath. In some aspects, the vaccine can be administered to multiple subjects simultaneously by immersing all of the subjects in the bath at the same time. In other aspects, the vaccine can be administered by intracoelomic (i.e.) injection of the subject.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a bacterial strain,” “a vector,” or “an antigen,” include, but are not limited to, mixtures or combinations of two or more such bacterial strains, vectors, or antigens, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a vaccine refers to an amount that is sufficient to achieve the desired improvement in the property modulated by the formulation component, e.g. achieving the desired level of control of disease for which the vaccine has been formulated. The specific level in terms of wt % in a composition required as an effective amount will depend upon a variety of factors including the method of administration, amount and type of subject organism, body weight of the subject organisms, and water or other environmental conditions for the organisms.

As used herein, the terms “optional” or “optionally” means that the subsequently described event or circumstance can or cannot occur, and that the description includes instances where said event or circumstance occurs and instances where it does not.

As used herein, “additive effect” can refer to an effect arising between two or more molecules, compounds, substances, factors, or compositions that is equal to or the same as the sum of their individual effects.

As used herein, “administering” can refer to any administration route, including but not limited to administration that is oral, topical, intravenous, subcutaneous, transcutaneous, transdermal, intramuscular, intra-articular, parenteral, intra-arterial, intradermal, intraventricular, intracranial, intracelomically, intralesional, or via bath immersion. The term “parenteral” includes subcutaneous, intravenous, intramuscular, and intracelomically by injections or infusion techniques.

As used interchangeably herein, “biocompatible,” “biocompatibility,” and “biologically compatible” refer to materials that are, with any metabolites or degradation products thereof, generally non-toxic to the recipient, and cause no significant adverse effects to the recipient. Generally speaking, biocompatible materials are materials which do not elicit a significant inflammatory or immune response when administered to a patient. In some embodiments, a biocompatible material elicits no detectable change in one or more biomarkers indicative of an immune response. In some embodiments, a biocompatible material elicits no greater than a 10% change, no greater than a 20% change, or no greater than a 40% change in one or more biomarkers indicative of an immune response.

As used herein, “composition” or “formulation” can refer to a combination of an active agent(s) and another compound or composition, inert (for example, a detectable agent or label) or active, such as an adjuvant.

As used herein, “control” can refer to an alternative subject or sample used in an experiment for comparison purposes and included to minimize or distinguish the effect of variables other than an independent variable. A control can be positive or negative.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “mitigate” can refer to reducing a particular characteristic, symptom, or other biological or physiological parameter associated with a disease or disorder.

As used herein, “pharmaceutical formulation” can refer to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable” can refer to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

As used herein, “pharmaceutically acceptable carrier or excipient” refers to a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used herein also includes both one and more than one such carrier or excipient. Pharmaceutically acceptable carriers include, but are not limited to, diluents, preservatives, binders, lubricants, disintegrators, swelling agents, fillers, stabilizers, and combinations thereof.

As used herein, “pharmaceutically acceptable salt” can refer to any salt derived from organic and inorganic acids of a compound described herein. Pharmaceutically acceptable salt also refers to a salt of a compound described having an acidic functional group, such as a carboxylic acid functional group, and a base. Pharmaceutically acceptable salt also includes hydrates of a salt of a compound described herein.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms including, but not limited to prevent infection by a pathogen such as A. hydrophila or a symptom thereof.

As used interchangeably herein, “subject,” “individual,” or “patient,” can refer to a vertebrate, in particular a fish such as a teleost fish, a catfish, tilapia, and the like.

The terms “sufficient” and “effective,” as used interchangeably herein, can refer to an amount (e.g. mass, volume, dosage, concentration, and/or time period) needed to achieve one or more desired result(s). For example, a therapeutically effective amount refers to an amount needed to achieve one or more therapeutic effects.

As used herein, “synergistic effect,” “synergism,” or “synergy” can refer to an effect arising between two or more molecules, compounds, substances, factors, or compositions that that is greater than or different from the sum of their individual effects.

As used herein, “therapeutic”, “treating”, “treat” and the like can refer to include partially or completely delaying, alleviating, mitigating or reducing the intensity of one or more attendant symptoms of a disease or condition including, but not limited to, infection by a virus or bacterial pathogen.

As used herein, “antigen” refers to a biomolecule capable of eliciting an immune response in a host. In some embodiments, the antigen may be a protein, or fragment of a protein, or a modified protein such as by glycosylation or a carbohydrate or a nucleic acid. Therefore, the antigen can be an immunogenic fragment of a protein or modified protein disclosed herein.

In an exemplary embodiment, the antigen elicits a protective immune response. As used herein, “protective” means that the immune response contributes to the lessening of any symptoms associated with infection of a host with the pathogen the antigen was derived from or designed to elicit a response against. For example, a protective antigen from Aeromonas may induce an immune response that helps to ameliorate symptoms associated with Aeromonas infection or reduce the morbidity and mortality associated with infection with the pathogen. The use of the term “protective” in this invention does not necessarily require that the host be completely protected from the effects of the pathogen.

It is not necessary that the vector comprise the complete nucleic acid sequence of the antigen. It is only necessary that the antigen sequence used be capable of eliciting an immune response. The antigen may be one that was not found in that exact form in the parent organism. For example, a sequence coding for an antigen comprising 100 amino acid residues may be transferred in part into a recombinant bacterium so that a peptide comprising only 75, 65, 55, 45, 35, 25, 15, or even 10, amino acid residues is produced by the recombinant bacterium. Alternatively, if the amino acid sequence of a particular antigen or fragment thereof is known, it may be possible to chemically synthesize the nucleic acid fragment or analog thereof by means of automated nucleic acid sequence synthesizers, PCR, or the like and introduce said nucleic acid sequence into the appropriate copy number vector.

In another alternative, a vector may comprise a long sequence of nucleic acid encoding several nucleic acid sequence products, one or all of which may be antigenic. In some embodiments, a vector may comprise a nucleic acid sequence encoding at least one antigen, at least two antigens, at least three antigens, or more than three antigens. These antigens may be encoded by two or more open reading frames operably linked to be expressed coordinately as an operon, wherein each antigen is synthesized independently. Alternatively, the two or more antigens may be encoded by a single open reading frame such that the antigens are synthesized as a fusion protein.

Additionally, the vectors may be designed for various types of antigen delivery systems. The system that is selected depends, in part, on the immune response desired. For example, if an antibody response is desired, then a Type II secretion system may be used. Examples of Type II secretion systems are well-known in the art. Alternatively, if a cytotoxic T lymphocyte (CTL) response is desired, then a Type III secretion system may be used. Type III secretion systems are known in the art. This type of antigen delivery system delivers the antigen to the cytoplasm of cells in the host to enhance induction of CTL responses. Yet another type of antigen delivery strategy that may be used is regulated delayed lysis of a bacterium in vivo to release protein antigen(s) and/or viral proteins. The viral proteins may include viral core particles with or without epitope fusion. Regulated antigen delivery systems are known in the art. See, for example, U.S. Pat. No. 6,780,405, hereby incorporated by reference in its entirety. In other embodiments, the antigen may be delivered to the cytosol of a host cell by lysis of the recombinant bacterium. Such lysis may be regulated as described herein.

Furthermore, antigen delivery is not limited to expression by plasmid vectors in the bacterium. Protective antigen sequences that encode antigens of the present disclosure can also integrated in chromosomal sites. Generally the chromosomal sites selected cause insertion of protective antigen-encoding sequences to be inserted into a chromosomal gene, often in replacement of an easily identifiable chromosomal gene. Selection of the chromosomal gene site for insertion is important. First of all, the absence of the inactivated chromosomal gene cannot be deleterious to the vaccine strain to decrease its invasiveness and ability to be highly immunogenic. Also, it is often useful to insert the antigen encoding sequence into a gene near the origin of chromosome replication since this increases gene copy number during growth of the bacterial vector and thus the amount of antigen synthesized to enhance induced immune levels. In some cases, the gene site for insertion is into a gene already inactivated for some other beneficial attribute of the vaccine vector. In all cases, suicide vector technologies are used for the insertion of antigen-encoding sequences into chromosomal sites.

“Plasmid copy number” as used herein refers to the average or expected number of copies of a given plasmid per host cell. A “high” plasmid copy number yields 3-5 μg of DNA per 1 mL LB culture of the host cells, while a “low” plasmid copy number yields 0.2-1 μg of DNA per 1 mL of LB culture of the host cells, with “medium” plasmid copy numbers occupying the space between high and low numbers. In one aspect, plasmid copy numbers as disclosed herein depend on the ori sequence of a given plasmid.

In an aspect, “regulated delayed attenuation” refers to a strategy for retaining immunogenicity of vaccines based on bacterial vectors. Vaccine vectors with regulated delayed attenuation can invade and colonize lymphoid tissues during early stages of infection due to one or more sugars (such as, for example, arabinose) provided in vitro during culture, but become attenuated in vivo as the sugars become unavailable.

As used herein, “regulated delayed antigen synthesis” (RDAS) refers to a vaccine strain that includes an antigen-encoding plasmid in vitro. In such a system, a LacI-repressible promoter such as, for example, P_{trc}, controls antigen gene expression by adding arabinose. When arabinose is no longer present (e.g. when the vaccine has entered target tissues in a host organism that does not express arabinose), antigen synthesis can begin.

“Regulated delayed lysis” as used herein is a safety feature for vaccines. A regulated delayed lysis system allows attenuated bacterial strains to colonize host tissues only if the araC P_{BAD} promoter is activated with an exogenous source of arabinose. In a regulated delayed lysis system, accumulated arabinose in bacterial cells is exhausted, and genes encoding bacterial cell wall enzymes become blocked, eventually resulting in cell lysis.

“Biological containment” as used herein refers to a strategy to prevent environmental escape of engineered live vaccine or drug delivery vehicles. Regulated delayed attenuation, regulated delayed antigen synthesis, and regulated delayed lysis in vivo can all be components of strategies for biological containment.

Unless otherwise specified, pressures temperatures referred to herein are based on standard conditions such as ambient temperature and atmospheric pressure (i.e., one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered Aspects, which should not be confused with the claims.
Aspect 1. A recombinant, attenuated, biologically-contained Edwardsiella bacterial vector displaying regulated delayed lysis attributes, the vector comprising a gene encoding at least one antigen from a fish pathogen;
wherein the at least one antigen or a DNA vaccine vector containing the gene encoding the at least one antigen induces expression of host genes for enhancement of innate immunity after entry of the antigen or DNA vaccine vector into a eukaryotic host cell.
Aspect 2. The recombinant attenuated biologically contained Edwardsiella bacterial vector of aspect 1, wherein the bacterial vector comprises an Edwardsiella piscicida strain.
Aspect 3. The recombinant attenuated biologically contained Edwardsiella bacterial vector of aspect 2, wherein the E. piscicida strain is derived from J118.
Aspect 4. The recombinant attenuated biologically contained Edwardsiella bacterial vector of aspect 1, wherein entry into the eukaryotic cell causes expression of one or more host cytokine genes.
Aspect 5. The recombinant attenuated biologically contained Edwardsiella bacterial vector of aspect 4, wherein the cytokine gene comprises a il-8, il-1β, tnf-α, il-6, or ifn-γ gene, or any combination thereof.
Aspect 6. The recombinant attenuated biologically contained Edwardsiella bacterial vector of aspect 5, wherein expression of the one or more host cytokine genes occurs in gills, kidneys, intestine, spleen, blood, or any combination thereof.
Aspect 7. The recombinant attenuated biologically contained Edwardsiella bacterial vector of aspect 1, wherein entry into a eukaryotic cell causes expression of genes encoding CD4-1, CD4-2, CD8-α, CD8-β, MHC-II, or any combination thereof.
Aspect 8. The recombinant attenuated biologically contained Edwardsiella bacterial vector of aspect 1, wherein the fish pathogen comprises a bacterial pathogen.
Aspect 9. The recombinant attenuated biologically contained Edwardsiella bacterial vector of aspect 1, wherein the fish pathogen comprises a viral pathogen.
Aspect 10. The recombinant attenuated biologically contained Edwardsiella bacterial vector of aspect 1, wherein the fish pathogen comprises a parasitic pathogen.
Aspect 11. The recombinant attenuated biologically contained Edwardsiella bacterial vector of aspect 8, wherein the bacterial pathogen comprises Aeromonas hydrophila.
Aspect 12. The recombinant attenuated biologically contained Edwardsiella bacterial vector of aspect 9, wherein the viral pathogen comprises tilapia lake virus (TiLV).
Aspect 13. The recombinant attenuated biologically contained Edwardsiella bacterial vector of aspect 11, wherein the antigen comprises outer membrane protein CC002501, AGM43135, AGM42919, AOKFG8, AOKGW8, AOKQ46, AOKQZ1, AOKIU8, AGH12866, a TonB domain of AOKFG8, an OMP domain of AOKFG8, a Plug domain of AOKFG8, or any combination thereof.
Aspect 14. The recombinant attenuated biologically contained Edwardsiella bacterial vector of aspect 12, wherein the TiLV gene encoding the at least one antigen and has at least 80% sequence identity with any one of SEQ ID NOs. 47-55.
Aspect 15. The recombinant attenuated biologically contained Edwardsiella bacterial vector of aspect 1, wherein the gene encoding the at least one antigen is incorporated into a plasmid.
Aspect 16. The recombinant attenuated biologically contained Edwardsiella bacterial vector of aspect 15, wherein the plasmid comprises G8R111, G8R114, or pYA4545.
Aspect 17. The recombinant attenuated biologically contained Edwardsiella bacterial vector of aspect 1, wherein the recombinant vector transiently colonizes at least one tissue type in a subject, and wherein colonization by the vector induces release of the at least one antigen.
Aspect 18. The recombinant attenuated biologically contained Edwardsiella bacterial vector of aspect 17, wherein the at least one tissue type comprises internal lymphoid tissues.
Aspect 19. The recombinant attenuated biologically contained Edwardsiella bacterial vector of aspect 18, wherein in the vector, at least one gene promoter has been replaced with an araC P_{araBAD} cassette to create a delayed attenuation phenotype.
Aspect 20. The recombinant attenuated biologically contained Edwardsiella bacterial vector of aspect 19, wherein the at least one gene promoter comprises a promoter for the fur gene, the crp gene, or both the fur and crp genes.
Aspect 21. The recombinant attenuated biologically contained Edwardsiella bacterial vector of aspect 19, wherein presence of the araC P_{araBAD} cassette induces vector dependence on arabinose.
Aspect 22. The recombinant attenuated biologically contained Edwardsiella bacterial vector of aspect 1, wherein the vector persists for about two weeks or less in vivo after administration to a subject.
Aspect 23. The recombinant bacterial vector of aspect 1, wherein the vector is sensitive to one or more antibiotics used to treat infections caused by Edwardsiella bacteria.
Aspect 24. A vaccine comprising the recombinant attenuated biologically contained Edwardsiella bacterial vector of any one of aspects 1-23.
Aspect 25. A method for generating an immune response to at least one pathogen in a subject, the method comprising administering to a subject the vaccine of aspect 24 to the subject.
Aspect 26. The method of aspect 25, wherein the subject is a fish in an aquaculture system.
Aspect 27. The method of aspect 26, wherein the subject is a fry, juvenile, adult, or spawning fish.
Aspect 28. The method of aspect 27, wherein the subject is a tilapia, catfish, trout, mullet, salmon, carp, or striped bass.
Aspect 29. The method of aspect 25, wherein the pathogen comprises tilapia lake virus (TiLV).
Aspect 30. The method aspect 25, wherein the pathogen comprises Aeromonas hydrophila.
Aspect 31. The method of aspect 25, wherein the vaccine additionally generates an immune response against at least one additional pathogen.
Aspect 32. The method of aspect 31, wherein the at least one additional pathogen comprises wild type Edwardsiella piscicida, Edwardsiella ictaluri, Edwardsiella hoshinae, Edwardsiella tarda, or any combination thereof.
Aspect 33. The method of aspect 25, wherein administration is carried out by bath immersion of the subject.
Aspect 34. The method of aspect 25, wherein the vaccine is administered by intracoelomic (i.e.) injection of the subject.
Aspect 35. The method of aspect 25, wherein administration is conducted once.
Aspect 36. The method of aspect 25, wherein the vaccine or vector is administered twice, wherein a booster administration occurs about six weeks after an initial administration.
Aspect 37. A method for preventing infection by a pathogen in at least one subject, the method comprising administering the vaccine of aspect 24 to the subject.
Aspect 38. The method of aspect 37, wherein the subject is a fish in an aquaculture system.
Aspect 39. The method of aspect 38, wherein the subject is a fry, juvenile, adult, or spawning fish.
Aspect 40. The method of aspect 39, wherein the subject is a tilapia, catfish, trout, mullet, salmon, carp, or striped bass.
Aspect 41. The method of aspect 37, wherein the pathogen comprises tilapia lake virus (TiLV).
Aspect 42. The method of aspect 37, wherein the pathogen comprises Aeromonas hydrophila.
Aspect 43. The method of aspect 37, wherein the vaccine additionally induces immunity against at least one additional pathogen.
Aspect 44. The method of aspect 43, wherein the at least one additional pathogen comprises wild type Edwardsiella piscicida, Edwardsiella ictaluri, Edwardsiella hoshinae, Edwardsiella tarda, or any combination thereof.
Aspect 45. The method of aspect 37, wherein the vaccine is administered by bath immersion of the subject.
Aspect 46. The method of aspect 37, wherein the vaccine is administered by intracoelomic (i.e.) injection of the subject.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Development of Strain as Basis for Conferring Biological Containment

Constructing vaccines that display biological containment to ensure inability of vaccine constructs to persist in tissues within a vaccinated individual and to not be able to survive in the environment, and especially in aqueous environments used for the bath immersion vaccination of fish, is of paramount importance. Described herein are the construction and characterization of E. piscicida strains with this attribute.

### Materials and Methods

Bacterial strains, plasmids, media, and reagents. All bacterial strains and plasmids used in this study are listed in Table 1. Bacterial strains were grown on Luria-Bertani (LB) agar or in LB broth. When necessary, media was supplemented with 10% sucrose, colistin sulfate (Col) (12.5 μg/ml), chloramphenicol (Cm) (25 μg/mL), and arabinose (0.2%). Growth of bacteria was determined spectrophotometrically and/or by serial dilution and plating. Oligonucleotides were from IDT (Coralville, IA). New England BioLabs restriction endonucleases and T4 ligase were used for cloning. For all PCR reactions, GoTaq DNA polymerase (Promega, catalog #M3008) was used. For plasmid DNA isolation and purification of gel fragments and PCR products, Qiagen products (Hilden, Germany) were used.

TABLE 1
Bacterial Strains and Plasmids

| | |
|---|---|
| Strain or plasmid | Relevant characteristics |

Strains
Escherichia coli

| | |
|---|---|
| χ7213 | thi-1 thr-1 leuB6 glnV44 fhuA21 lacY1 recA1 |
| | RP4-2-Tc::Mu λpir ΔasdA4 Δzhf-2::Tn10 |

E. piscicida

| | |
|---|---|
| J118 | Wild-type E. piscicida EIB202, highly virulent, |
| | fish isolated, Col^{r} |
| χ16016 | J118 derivative; ΔP_{murA180}::TT araC P_{araBAD} murA |

Plasmids

| | |
|---|---|
| pRE112 | Suicide vector; sacB mobRP4 R6K ori; Cm^{r} |
| pYA3700 | Cloning vector containing TT araC P_{araBAD} cassette |
| pG8R8025 | ΔP_{murA180}::TT araC P_{araBAD} |
| | murA, pRE112 |

Fish husbandry. In this study, all procedures and treatment of fish were approved by the Institutional Animal Care and Use Committee (IACUC), University of Florida. Channel catfish (Ictalurus punctatus) fingerlings (2.5±0.5 g) deemed clinically healthy were acclimated to laboratory conditions for two weeks after purchase from Osage Catfisheries, MO, USA. Fish were housed in 40 L holding tanks (35 fish/tank) at a water temperature of 26±2° C. Conditioned reverse osmosis (RO) water was used with a conductivity between 300-400 μS and pH between 7.0 and 7.4 achieved by adding instant sea salt and sodium bicarbonate. Fish were fed with commercial pellets twice a day and maintained on a 14 h light:10 h dark photoperiod. Fish were sedated with SYNCAINE® (MS 222) (40 mg/L) prior to being i.e. injected with either PBS or the vaccine.

Sequence analysis. The unrooted phylogenetic tree of MurA was constructed by the “neighbor-joining” method using MEGA 6.0 software. Publicly available MurA protein sequences were retrieved from NCBI GenBank database. Salmonella enterica (GenBank: EB14772669.1), Salmonella enterica subsp. enterica serovar Typhimurium (EDL2719154.1), Escherichia coli (EF04224361.1), Shigella flexneri (EFZ8852790.1), Aeromonas hydrophila (WP_025328540.1), Aeromonas salmonicida (HBL03892.1), Edwardsiella hoshinae (WP_024524569.1), Edwardsiella tarda (SPW31063.1), Edwardsiella ictaluri (WP_015870014.1), Yersinia pestis (PCN66959.1), Vibrio anguillarum (WP_026028554.1), Vibrio cholerae (WP_033932926.1), Staphylococcus aureus (NFZ33873.1), and Bacillus cereus (AUZ28172.1) were used. The three-dimensional (3D) structures of the E. piscicida, E. ictaluri, and S. typhimurium MurA protein were predicted by using Phyre2 web portal.

Construction of E. piscicida deletion-insertion mutation of ΔP_{murA}. To construct the E. piscicida strain with the ΔP_{murA}180::TT araC P_{araBAD} murA (hereinafter abbreviated ΔP_{murA}) deletion-insertion mutation, a 595-bp DNA fragment containing the region upstream of the murA promoter was PCR amplified using the E. piscicida (J118) genomic DNA as a template with primers MurA1-SphI and MurA2-BglII (Table 2). The PCR-amplified fragment was digested with SphI and BglII, and cloned into the SphI-BglII site of vector pYA3700, which lies just upstream of araC gene in the plasmid. Primer pYA3700-FW, which binds to the region just upstream of the HindIII-SphI site in pYA3700, and primer MurA2-BglII were used to screen plasmid isolates for inserts in the correct orientation. PCR fragments of 499 bp were amplified from the E. piscicida (J118) genomic DNA using upstream primer MurA3-KpnI, which contains the modified Shine-Dalgarno (SD) sequence “AGGAGG,” and the downstream primer MurA4-EcoRI. The PCR fragments were digested with KpnI and EcoRI, and inserted into the KpnI-EcoRI site of the intermediate plasmid described above. The resulting construct was confirmed by DNA sequence analysis. Then, a 2441-bp DNA fragment, including araC P_{araBAD} and murA 5′ and 3′ flanking regions, was amplified from the intermediate plasmid by using primers MurA1-SphI and MurA5-XmaI (Table 2). The amplified product was cloned into the SphI-XmaI site of the suicide vector pRE112. The recombinant plasmids were screened by PCR and underwent restriction digestion with SphI-XmaI enzymes, and the resultant plasmid was named pG8R8025. To construct the E. piscicida ΔP_{murA180}::TT araC P_{araBAD} murA mutant, the suicide plasmid pG8R8025 was conjugationally transferred from Escherichia coli χ7213 to E. piscicida wild-type strains J118. Strains containing single-crossover plasmid insertions were isolated on LB agar plates containing Col and Cm. Loss of the suicide vector after the second recombination between homologous regions (i.e., allelic exchange) was selected for by using the sacB-based sucrose sensitivity counterselection system. The colonies were screened for CmS, Colr and growth only in presence of arabinose. Colonies were screened by PCR using primers MurA1-SphI and MurA5-XmaI (Table 2). The resultant E. piscicida containing ΔP_{murA180}::TT araC P_{araBAD} murA mutation (hereafter referred to as ΔP_{murA}) was named as χ16016. The ΔP_{murA} deletion-insertion mutant (χ16016) was confirmed by PCR and DNA sequencing.

TABLE 2
Primers used for ΔP_{murA} deletion
insertion mutation

| | | SEQ |
|---|---|---|
| | | ID |
| Primer | Sequence (5′ → 3′) | NO. |
| MurA1-SphI | (F) CATGCATGCCGGGCATCACGT | 1 |
| | GTGTGGATATC | |
| MurA2-BgIII | (R) CATAGATCTTCACAGACCGCT | 2 |
| | CAGCTTGCG | |
| MurA3-KpnI | (F) CATGGTACCAGGAGGAATTAA | 3 |
| | ATGGATAAATTTCGT | |
| MurA4-EcoRI | (R) CCGGAATTCCGCTGACCTTGT | 4 |
| | CCATCACGATA | |
| MurA5-XmaI | (R) CATCCCGGGCGCTGACCTTGT | 5 |
| | CCATCACGATA | |
| pYA3700 | (F) ATAACCTTTCATTCCCAGCGG | 6 |

Growth curve analysis. Herein are compared the growth curves of E. piscicida wild-type strain J118 and the ΔP_{murA} mutant strain with and without arabinose. Overnight standing cultures (OD₆₀₀˜0.6) of E. piscicida strains were diluted 1:100 into prewarmed LB or LB plus arabinose (0.2% or 0.1%) broth and incubated at 30° C. with shaking at 180 RPM. The OD₆₀₀ of the cultures was measured every 60 min. The growth curves were calculated using the automated growth curve device Bioscreen C (Growth Curves USA, Piscataway, NJ).

Attachment, internalization, and intracellular replication assays. To identify the effect of ΔP_{murA} mutation on virulence, the attachment, internalization and intracellular replication ability of χ16016 with wild-type E. piscicida J118 have been compared. Attachment, internalization and intracellular replication assays using counts of viable bacteria were performed as described previously.

Attachment assay: EPC cells were infected by J118 or χ16016, in the presence or absence of arabinose, at a multiplicity of infection (MOI) of 10 and were incubated for 1 h to reach an adequate infection level. The monolayers were gently washed three times with cell culture medium to remove nonadherent E. piscicida. They were then lysed with 1% Triton X-100 in PBS and serial 10-fold dilutions of the cell lysates were plated on LB agar plates (with or without arabinose) to determine the level of adherence.

Internalization assay: A gentamicin invasion assay was performed to assess the presence of internalized E. piscicida. After infection, EPC cells were washed three times and were incubated with cell culture media supplemented with 100 μg/mL gentamicin for 1 h at 30° C. in a 5% CO₂ incubator. After gentamicin treatment, cells were washed with PBS to remove the gentamicin and plated as described above.

Replication assay: After gentamicin treatment, cells were incubated with growth media containing 5 μg/mL of gentamicin to eliminate extracellular E. piscicida. The intracellular bacterial population was assayed two and four hours later. Cells were washed three times with PBS, lysed and intracellular E. piscicida were calculated as described above.

Activation of TLRs and NLRs by E. piscicida ΔP_{murA} strain. It was hypothesized that RAEVs with the regulated-delayed lysis attribute could be used as an adjuvant to activate innate immunity by delivering highly conserved microbial structures, including lipopolysaccharide, flagella, CpG DNA, lipoprotein, and peptidoglycan (PGN) fragments (diaminopimelic acid (DAP) and muramyl dipeptide (MDP)). The detection of conserved microbial motifs relies on the two major classes of PRRs, including toll-like receptors (TLRs) and NOD-like receptors (NLRs). To investigate the activation of TLRs and NLRs by χ16016, HEK-Blue cells expressing mouse TLR4, TLR5, TLR8, TLR9, NOD1 and NOD2 (all from InvivoGen) were used with a NFκ B-inducible secreted embryonic alkaline phosphatase (SEAP) reporter gene. A previously described protocol was followed. In brief, cells were maintained in DMEM growth medium at 37 0C in 5% CO₂. Cells were cultured in the presence of selective antibiotics, as recommended by the manufacturer, and passaged twice a week at 80-90% confluency. Bacterial cells were grown in LB broth with arabinose (χ16016) or without arabinose (J118) to an OD₆₀₀ of 0.8. Bacterial cultures were sedimented at room temperature and resuspended in tissue culture medium. Approximately 1×10⁵ HEK-Blue cells were suspended in 200 μL HEK-Blue™ Detection medium (Invivogen) and were mixed with bacterial cells at a MOI of 1 in 96-well cell culture plates (Corning). Uninfected HEK-Blue cells with HEK-Blue™ detection medium was used as a control. After 3, 6, 12 and 24 h of incubation at 37° C. in 5% CO₂, SEAP activity was determined at 655 nm according to the manufacturer's recommendations (InvivoGen). All samples were measured in triplicate. TLR and NLR stimulation were expressed relative to the level of SEAP activity of uninfected control cells.

Colonization and lysis of χ16016 in catfish tissues. Catfish fingerlings were vaccinated with χ16016 via bath immersion for 2 hours with 1×10⁶ CFU/mL. Kidneys and intestines were collected from fish at days 1, 2, 3 and 4 post-vaccination (five fish at each time point). The tissues were homogenized in 200 μL of BSG and a 10-fold serial dilution of each sample was plated on LB agar plates containing colistin sulfate (Col) (12.5 μg/mL) and 0.2% arabinose. The plates were incubated at 30° C. for 48 h and the colonies were counted. The data consisted of a combination of three independent assays.

For the lysis study, catfish fingerlings were i.e. injected with χ16016 or J118 with a dose of 1×10³ CFU/fish. Kidneys were collected from fish every other day up to 16 days post-vaccination (five fish at each time point). The tissues were homogenized in 200 μL of BSG and a 10-fold serial dilution of each sample was plated on LB agar plates containing colistin sulfate (Col) (12.5 μg/mL) and 0.2% arabinose. The data consisted of a combination of three independent assays. Differences between two groups were analyzed by two-way ANOVA, where asterisks (*) indicate a significant difference (**P<0.01, ****P<0.0001).

Determination of lethal dose 50 (LD₅₀) χ16016 by bath immersion and i.e. injection. To determine the LD₅₀ of χ16016, ten-fold serial dilutions of E. piscicida were performed, fresh cultures were made in sterile BSG and the concentration of bacteria was determined using the spread-plate method. Fish were i.e. injected with a dose of 100 μl of BSG containing different concentrations of CFU per fish (Tables 3 and 4). There were 10 fish in each group (two replicate tanks). Mortality was documented daily over a 21-day period, and the Reed and Muench method was used to calculate the LD₅₀ values. For the bath immersion, fish were immersed in tank water containing specific concentrations of bacteria ranging from 1×10⁴, 1×10⁶, and 1×10⁸ CFU/mL and there were 10 fish in each group (two replicate tanks). After 2 h, the fish were removed from the solution and placed into their respective original tanks.

TABLE 3
LD₅₀ Study of E. piscicida wild-type and
vaccine strains in catfish by i.c. injection

| | | | | | |
|---|---|---|---|---|---|
| Dose | | | | Mortality | |
| CFU/fish | mortality | deaths | survivals | % | LD₅₀ |

J118 (Wild-Type E. piscicida)

| | | | | | |
|---|---|---|---|---|---|
| 9 × 10² | 1/10 | 1 | 9 | 5 | 1.7 × 10⁴ |
| 9 × 10³ | 4/10 | 4 | 6 | 38 | |
| 9 × 10⁴ | 8/10 | 8 | 2 | 87 | |
| 9 × 10⁵ | 10/10 | 10 | 0 | 100 | |
| 9 × 10⁶ | 10/10 | 10 | 0 | 100 | |

χ16016 (ΔP_{murA180}::TT araC P_{araBAD} murA)

| | | | | | |
|---|---|---|---|---|---|
| 4 × 10⁴ | 0/10 | 0 | 10 | 0 | 5 × 10⁶ |
| 4 × 10⁵ | 0/10 | 0 | 10 | 0 | |
| 4 × 10⁶ | 4/10 | 4 | 6 | 40 | |
| 4 × 10⁷ | 10/10 | 10 | 0 | 100 | |
| 4 × 10⁸ | 10/10 | 10 | 0 | 100 | |

TABLE 4
LD₅₀ Study of E. piscicida wild-type and
vaccine strains in catfish by bath immersion
J118 (Wild-Type E. piscicida) CFU/mL

| | | | | | |
|---|---|---|---|---|---|
| 9 × 10⁵ | 0/10 | 0 | 10 | 0 | 4.8 × 10⁷ |
| 9 × 10⁶ | 2/10 | 2 | 8 | 20 | |
| 9 × 10⁷ | 10/10 | 10 | 0 | 100 | |

χ16016 (ΔP_{murA180}::TT araC P_{araBAD} murA)

| | | | | | |
|---|---|---|---|---|---|
| 4 × 10⁶ | 0/10 | 0 | 10 | 0 | — |
| 4 × 10⁷ | 0/10 | 0 | 10 | 0 | |
| 4 × 10⁸ | 0/10 | 0 | 10 | 0 | |

Evaluation of anti-E. piscicida IgM in caffish serum and skin mucus by enzyme-linked immunosorbent assay (ELISA). To assay antibodies in catfish serum and skin mucus, ELISA was performed using E. piscicida LPS following the protocol described previously. Polystyrene 96-well flat-bottom microtiter plates (Dynatech Laboratories Inc., Chantilly, Va.) were coated with 100 μL (in each well) of E. piscicida LPS (100 ng/well) in sodium carbonate-bicarbonate coating buffer (pH 9.6). The coated plates were incubated at 400 overnight. Free binding sites were blocked by adding 300 μL of 5% bovine serum albumin (BSA) in each well and plates were incubated for 1 h at room temperature. After washing, 100-μL of diluted catfish serum/mucus samples from control and vaccinated fish were added to individual wells in duplicate. 100 μL of sterile PBS was added to the blank control wells and incubated for 2 h at 37° C. After washing, mouse anti-catfish IgM monoclonal antibody (Aquatic Diagnostics Ltd, UK) was diluted 1:100 in PBS and 100 μL was added to each well and incubated for 1 h at room temperature. Biotinylated goat anti-mouse IgG (Southern Biotechnology Associates, Birmingham, AL) was diluted with 1% BSA and 100 μL was added to each well and incubated for 1 h at room temperature. After incubation of wells with a streptavidin-alkaline phosphatase conjugate (Southern Biotechnology) for 1 h at 37° C., p-nitrophenyl phosphate (PNPP, Thermo Fisher Scientific) was added for color development. The optical density (OD) units were read at 405 nm using an automated ELISA plate reader (model EL311SX; Biotek, Winooski, VT).

Immune protection against wild-type E. piscicida challenge. To assess the immune protection provided by χ16016 against wild-type E. piscicida challenge, 90 catfish fingerlings were vaccinated with χ16016 via bath immersion for 2 hours with 1×10⁶ CFU/mL and transferred to the original tanks. The same number of catfish fingerlings were kept for the control (PBS) group. Both the vaccinated and control group fish were split into triplicate tanks (n=30 fish/tank). Fish were booster vaccinated with either χ16016 (dose of 5×10⁶ CFU/mL) or PBS (control) via bath immersion as described above. Both vaccinated and PBS control fish were i.e. challenged with 3×10⁵ CFU of J118 per fish. Fish were monitored up to 21 days after challenge, mortalities were recorded and data were represented as percent survival. At the end of the 21-day challenge, all surviving fish were euthanized.

RNA isolation, first-strand cDNA synthesis, and quantitative real-time PCR. Total RNA was extracted from gills, kidneys, intestines, spleens and blood using Trizol reagent (Invitrogen, Carlsbad, CA, USA). RNA concentration was quantified using UV-spectroscopy and OD260/OD280 ratios between 1.8 and 2.0 were considered sufficient for analysis. The integrity of the RNA was assessed by observing the band intensity of 28 and 18S ribosomal RNA in 1% agarose gels. Total RNA was reverse-transcribed by using Thermo Scientific™ RevertAid™ Premium First Strand cDNA Synthesis Kit (Catalog #FERK1622). The cDNA synthesis was confirmed by PCR amplification of 18S rRNA gene and the synthesized cDNA was kept at −80° C. until further analysis.

Quantitative real-time PCR (qRT-PCR) analyses of the target genes il-8, il-1β, tnf-α, il-6, ifn-γ, cd4-1, cd4-2, cd8-α, cd8-β, mhc-ii and the reference gene 18S rRNA were performed (Table 5). qRT-PCR analysis was performed using 10 μL reaction volume containing 1 μl cDNA template, 0.25 μL of FW and RV primers (2.5 mM each), 5 μL of 2× PowerUp™ SYBR™ Green Master Mix (Thermofisher Catalog #A25742) and 3.5 μL of PCR grade deionized water in a Quantstudio 3 thermocycler (Applied Biosystems). The qRT-PCR was performed in triplicate wells under the following conditions: 95° C. for 10 min, followed by 45 cycles of 94° C. for 10 s, 58° C. for 10 s, and 72° C. for 10 s and a final step of 72° C. for 5 min. Negative control reactions were kept without the template (cDNA). The integrity of the amplified products was assessed via melting curve analysis using the software supplied with the instrument. The relative expression ratios were obtained by normalizing expression of the target gene (mean crossing point (cp) deviation by that of a nonregulated reference gene encoding 18S rRNA following the 2^{-ΔΔCT} method). The results were expressed as mean±standard error (bars) from three separate experiments. Differences between uninfected (control) and infected groups were analyzed by two-way ANOVA, where asterisks (*) indicate significant difference (*P<0.05, **P<0.01, ***P<0.001, ****P<0.0001), with respect to the control group. Primers are shown in Table 5.

TABLE 5
Primers used for qRT-PCR assay

| | | PCR | | SEQ |
|---|---|---|---|---|
| | | product | | ID |
| Gene | Sequence 5′-3′ | size in bp | GenBank # | NO. |
| 18S | F-GAGAAACGGCTACCACATCC | 128 | AF021880.1 | 7 |
| rRNA | | | | |
| | R-GATACGCTCATTCCGATTACAG | | | 8 |
| CD4-1 | F-GGGGATTTTTGCACAGTTCGG | 243 | DQ435305.1 | 9 |
| | R-TCCACAAGGTGATGCTGTTCA | | | 10 |
| CD4-2 | F-TGAACATCGGTGTTGTTGGTC | 238 | DQ435304.1 | 11 |
| | R-TCCTGTTCAGTGAAGGTGACTT | | | 12 |
| CD8-α | F-TCGTACAGCAAGACTAAGAAATCA | 198 | HQ446239.1 | 13 |
| | R-TTTGATTGTTGTTGGGACTGTCT | | | 14 |
| CD8-β | F-CTTAAGAACAGAGACACACCGGA | 299 | HQ446240.1 | 15 |
| | R-ACACGCTTTGGATTTTTCACTG | | | 16 |
| IL-1β | F-TGCTGGAAAGGTACTTTGACAG | 296 | DQ157743.1 | 17 |
| | R-GCACTTCAAATTGATCACCATGC | | | 18 |
| IL-8 | F-GCTGCCCTTGATCATCTTTGC | 219 | AY145142.1 | 19 |
| | R-GGATCCAGGCAGACCTTCATTC | | | 20 |
| IFN-γ | F-TACTGGACATGAGATTCACAACCT | 257 | NM_001200217.1 | 21 |
| | R-CTTTCACACTTTCCTGGAGTTC | | | 22 |
| MHC-II | F-CACTGAAGCACAGATCAAACACC | 236 | AF103002.2 | 23 |
| | R-CTGTTCTGTACACTCCTAGGTTGG | | | 24 |
| TNF-α | F-TCTTCAGGAGTTTGGAGAACCTG | 255 | AJ417565.2 | 25 |
| | R-CAGTTTCAAGCCTGAAGAGAAGG | | | 26 |
| IL-6 | F-GAAACATCTGGAGTCGAGCTGC | 214 | XM_017455306.1 | 27 |
| | R-GACATTTTTGCGGGCTGAAGTC | | | 28 |

### Results

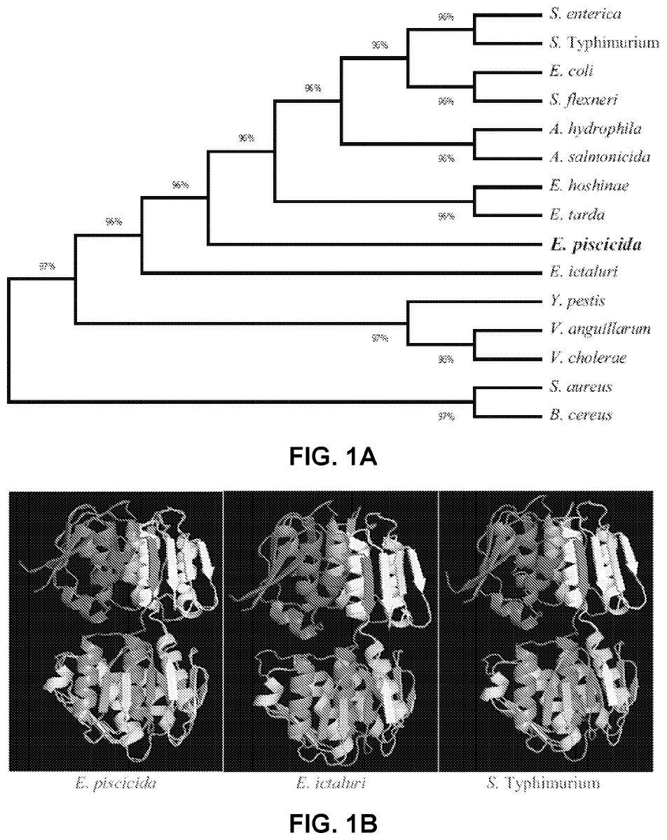

Sequence Analysis, phylogenetic relationship, and 3D structure of MurA. The E. piscicida EIB202 (J118) murA open reading frame consists of 1257 base pairs (bp) that encode a putative 418 amino acid (aa) residue protein with an estimated molecular mass of 44.5 kilodaltons (kDa). To analyze the evolutionary relationships between some bacterial MurA proteins, a phylogenetic tree was constructed using amino acid sequences (FIG. 1A). The results revealed that the MurA proteins of Edwardsiella species share high amino acid sequence identity, particularly those in E. ictaluri (98.80), E. hoshinae (97.37), and E. tarda (97.13). In the phylogenetic tree, the MurA proteins in E. piscicida and E. ictaluri form individual clusters. E. hoshinae and E. tarda MurA fall in the same cluster and are separated from other Edwardsiella species. S. typhimurium and S. enterica MurA proteins shared 87.88% sequence identity with E. piscicida MurA protein, and form a separate cluster from Edwardsiella species.

E. piscicida, E. ictaluri, and S. typhimurium MurA 3D structures were predicted by using the Phyre2 web portal. These results indicate that the MurA 3D structure is relatively conserved among these bacterial species. The MurA protein in E. piscicida, E. ictaluri, and S. typhimurium all have 14 α-helices and 21 β-sheets and thus share high sequence homology (FIG. 1B).

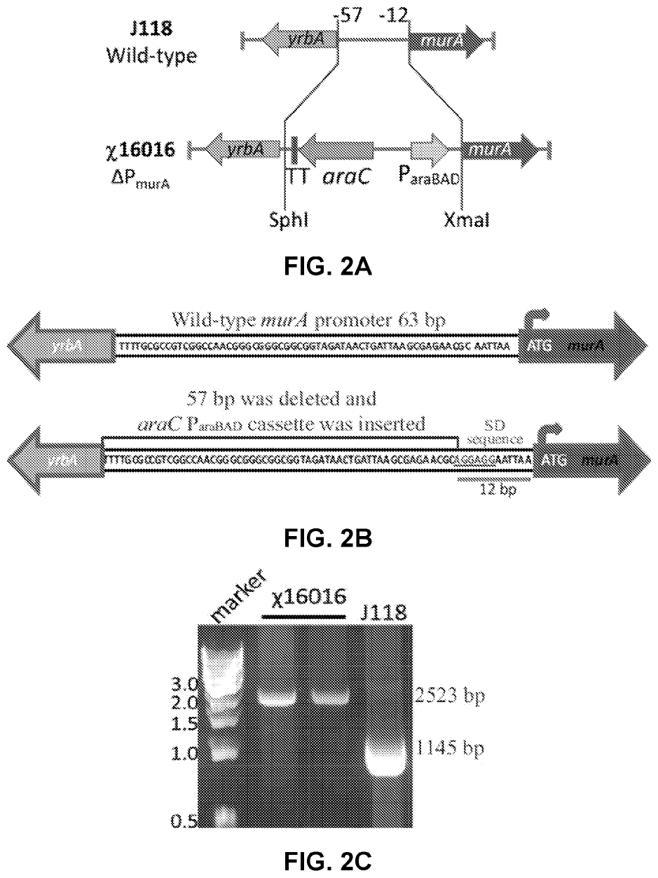

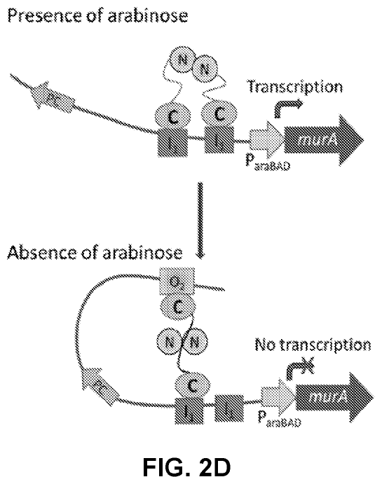

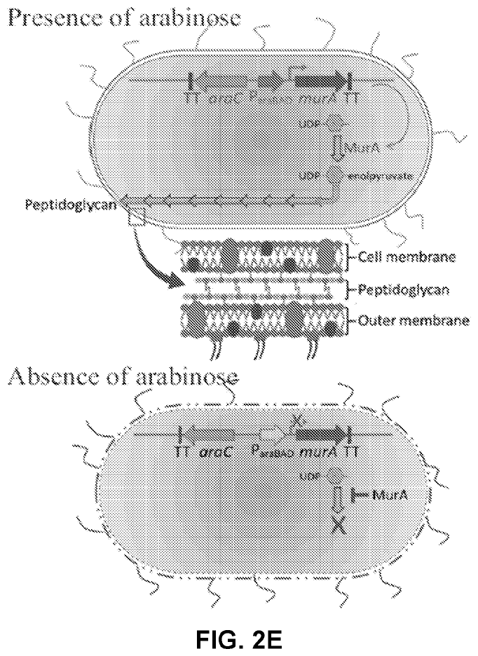

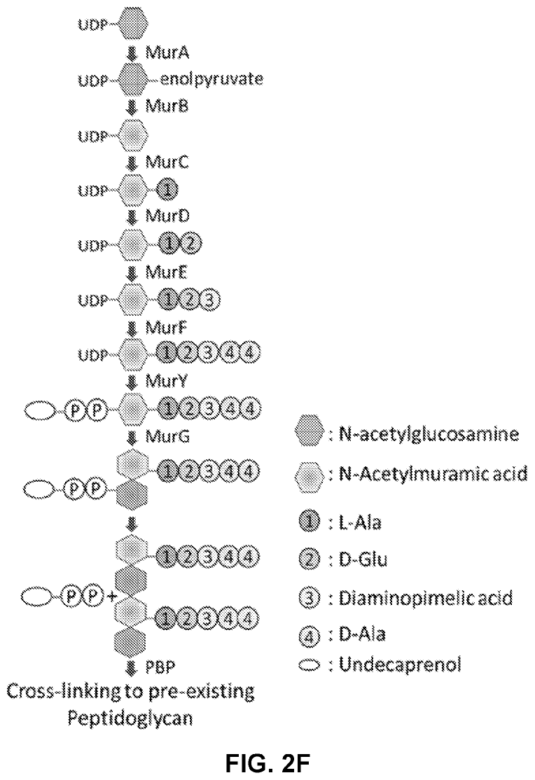

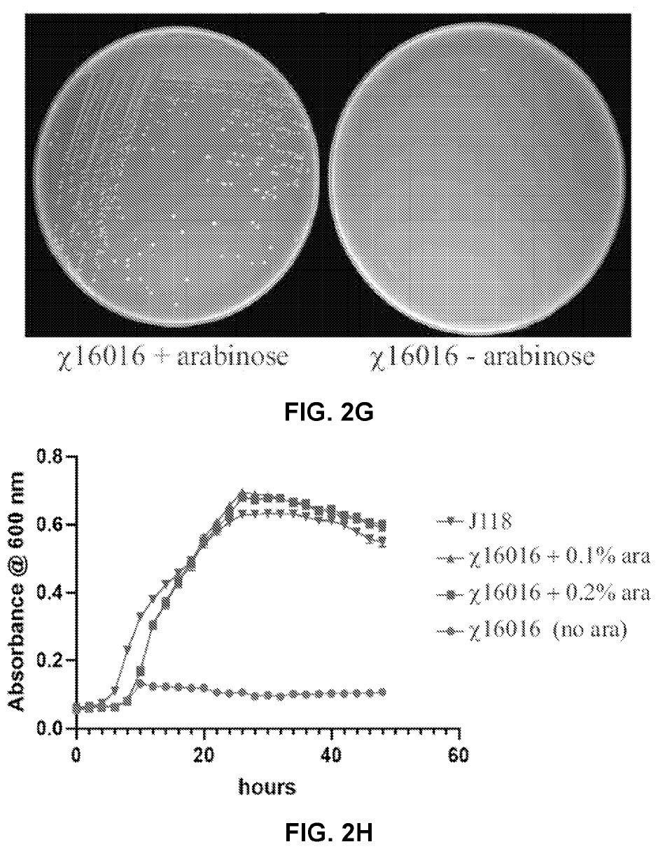

Construction and characterization of E. piscicida murA mutant. A conditional-lethal murA mutant strain was constructed by replacing the chromosomal murA wild-type promoter of 57 bp (from −12 to −57 bp upstream of the start codon) with an araC P_{araBAD} promoter of 1335 bp. A modified Shine-Dalgarno (SD) sequence “AGGAGG” was introduced at −6 bp upstream of the murA start codon. The ΔP_{murA180}::TT araC P_{araBAD} murA mutation was introduced into the wild-type E. piscicida strain J118, and the resultant strain was named χ16016. The chromosomal structure and sequence of the promoter region of the wild-type and χ16016 mutant strains are illustrated in FIGS. 2A-2B. The ΔP_{murA} deletion-insertion mutant (χ16016) was confirmed by PCR with primers MurA1-SphI and MurA5-XmaI. The replacement of the 57-bp wild-type promoter with the araC P_{araBAD} promoter resulted in a DNA band larger in size than the wild-type sequence, which shows a smaller band (FIG. 2C).

Principle of arabinose-regulated MurA synthesis. In the presence of arabinose, the AraC protein alters its conformation and forms a dimer that binds to I1 and I2 sites. This conformational change stimulates transcription of murA (FIG. 2D). In the absence of arabinose, the AraC protein dimer binds to the O2 and I1 regulatory regions on the araC P_{araBAD} promoter, generating a DNA loop that represses transcription of the araC P_{araBAD} promoter and inhibits MurA protein synthesis (FIG. 2D).

The bacterial peptidoglycan biosynthesis pathway is specified by a series of enzymes that have been widely studied. MurA is involved in the first step of peptidoglycan synthesis within the bacterial cytoplasm, generating enolpyruvyl uridine diphosphate (UDP)-N-acetylglucosamine (GlcNAc) (FIGS. 2E-2F). Since the product of the murA gene is a phosphorylated sugar that cannot be taken up by bacteria, murA deletion is lethal. Therefore, it is necessary to create a conditional-lethal murA mutation by replacing the chromosomal murA promoter with the araC P_{araBAD} activator-promoter. The ΔP_{murA180}::TT araC P_{araBAD} murA mutation was introduced into wild-type E. piscicida J118 to yield χ16016, and the phenotype of χ16016 was verified by growth in media with or without arabinose. In the absence of arabinose, the transcription of the murA gene was inhibited and χ16016 was unable to grow. In the presence of arabinose, the growth was shown to be similar to that of the J118 (wild type) (FIGS. 2G-2H).

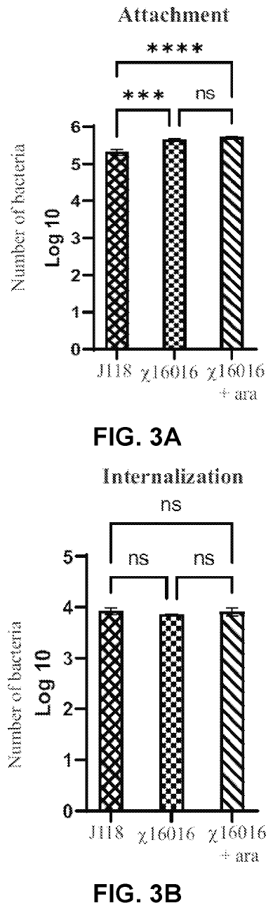

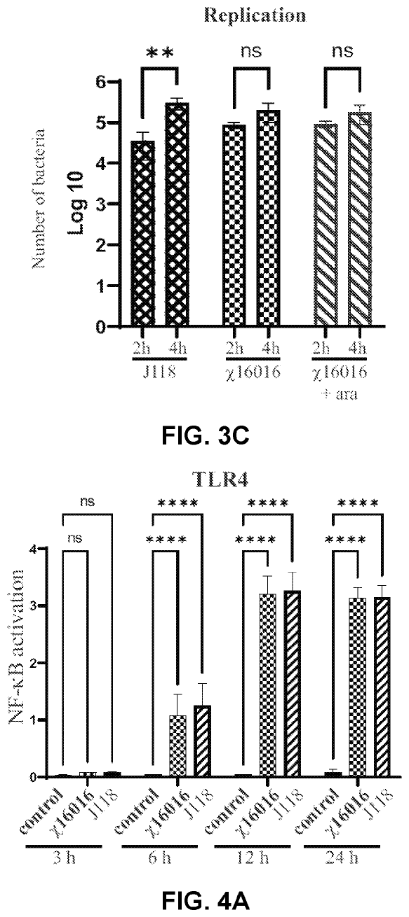

Attachment, internalization, and persistence of E. piscicida wild-type and, ΔP_{murA} (χ16016) strains in fish cell line. The numbers of E. piscicida wild-type and mutant strain cells that attached, internalized and replicated in fish cell lines were assessed. These results indicated that both wild-type and mutant strains proficiently attached to the EPC cells. However, the number of J118 that attached successfully was significantly lower compared to χ16016 with or without arabinose. There was no significant difference in adherence ability between J118 and χ16016 with or without arabinose (FIG. 3A). In the internalization study, there was no significant difference between J118 and χ16016 with or without arabinose (FIG. 3B). There was not a significant increase in bacterial population between 2 h and 4 h of incubation for χ16016 with or without arabinose, though there was a significant increase for J118 (FIG. 3C). These results suggest that the murA mutation somehow affects the intracellular replication in EPC cells.

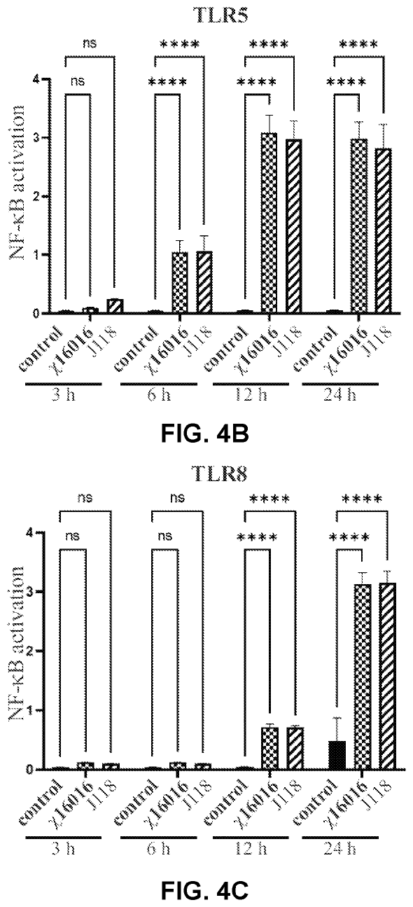

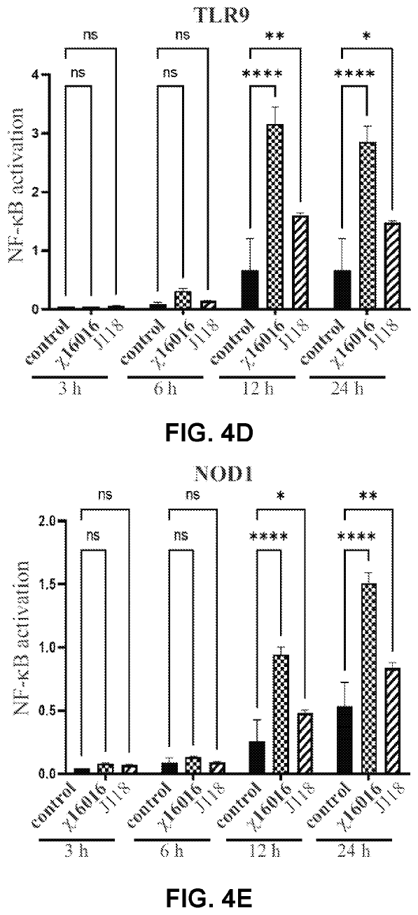

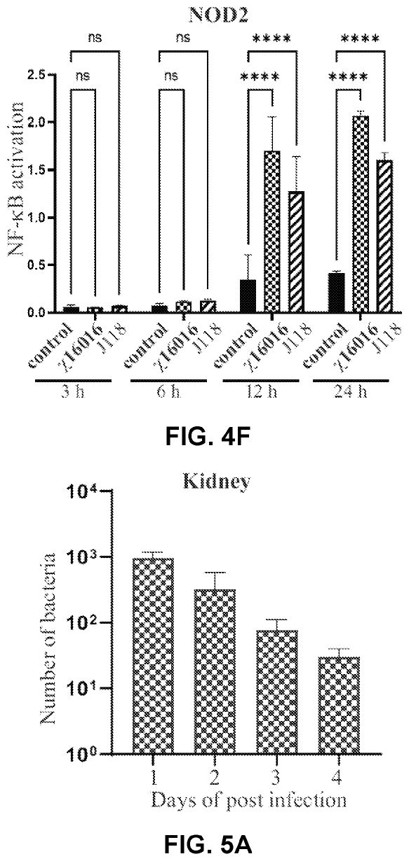

Activation of NF-κB pathway through TLRs and NLRs by E. piscicida strains. The transcription factor NF-κB plays important roles in both innate and adaptive immune responses to various microbial infections, as well as in regulating immune defense. TLR and NLR signaling leads to NF-κB activation and initiates inflammatory and antimicrobial responses against microbial infection. The role that wild-type E. piscicida (J118) and ΔP_{murA} strains play in TLR and NLR signaling leading to NF-κB activation was analyzed by using HEK-Blue™-mTLR4, mTLR5, mTLR8, mTLR9, mNOD1 and mNOD2 cells. These results indicated that the ΔP_{murA} mutant χ16016 was able to stimulate the TLR and NLR signaling pathways, thereby activating NF-κB at 6, 12 and 24 h post infection. The levels of induction of TLR4-, TLR5-, and TLR8-mediated NF-κB by χ16016 was similar to that of J118 (FIGS. 4A-4C). Activation of TLR9-mediated NF-κB by χ16016 was significantly higher compared to that of J118 (FIG. 4D). χ16016 also induced NOD1- and NOD2-mediated NF-κB to a higher degree than J118 (FIGS. 4E-4F).

Determination of lethal dose 50 (LD₅₀) dose of wild-type and vaccine candidate by i.e. injection and bath immersion. These results showed that the vaccine strain χ16016 injected by i.e. route had a significantly higher LD₅₀ (5×10⁶ CFU) compared to the wild-type strain (1.7×10⁴ CFU) in catfish (Table 3). The LD₅₀ of the wild-type strain J118 by bath immersion was 4.8×10⁷ CFU/mL. However, there were no mortalities in catfish bath-immersed with a high dose (4×10⁸ CFU/mL) of χ16016 (Table 4).

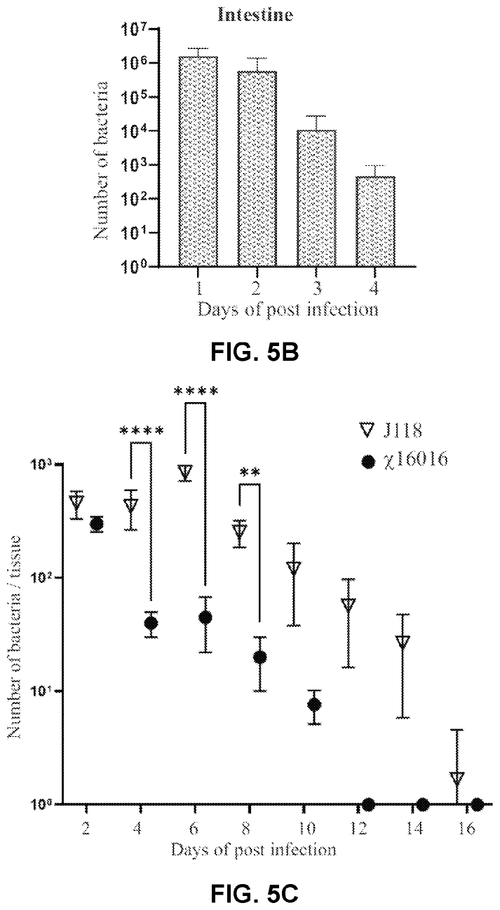

Colonization and lysis of χ16016 in catfish tissues. The ideal live attenuated bacterial vaccine should be sufficiently attenuated, delivered through a mucosal route and be able to colonize fish tissues efficiently to elicit a potent immune response. To test the colonization efficiency of the ΔP_{murA} mutant delivered through bath immersion into the catfish tissues, kidneys and intestines were collected at day 1, 2, 3 and 4 from the catfish immunized with χ16016 by bath immersion. Significant numbers of bacteria colonized both the kidneys and intestines at 1, 2, 3, and 4 days post-vaccination (FIGS. 5A-5B). This result indicates that the χ16016 successfully colonized and disseminated into different catfish tissues following bath immersion. To evaluate the cell lysis of χ16016 in fish tissue, catfish were i.e. injected with χ16016 or JA18. Kidneys were collected from fish every other day up to 16 days post-vaccination. Both J118 and λ16016 significantly colonized catfish kidneys 2 days after vaccination. The number of χ16016 decreased with the progression of time and was completely lysed at day 12 in catfish tissues. The wild-type strain J 18, however, persisted at day 16 (FIG. 5C). This ensures complete safety of the vaccine and demonstrates the biocontainment property of the ΔP_{murA} strain.

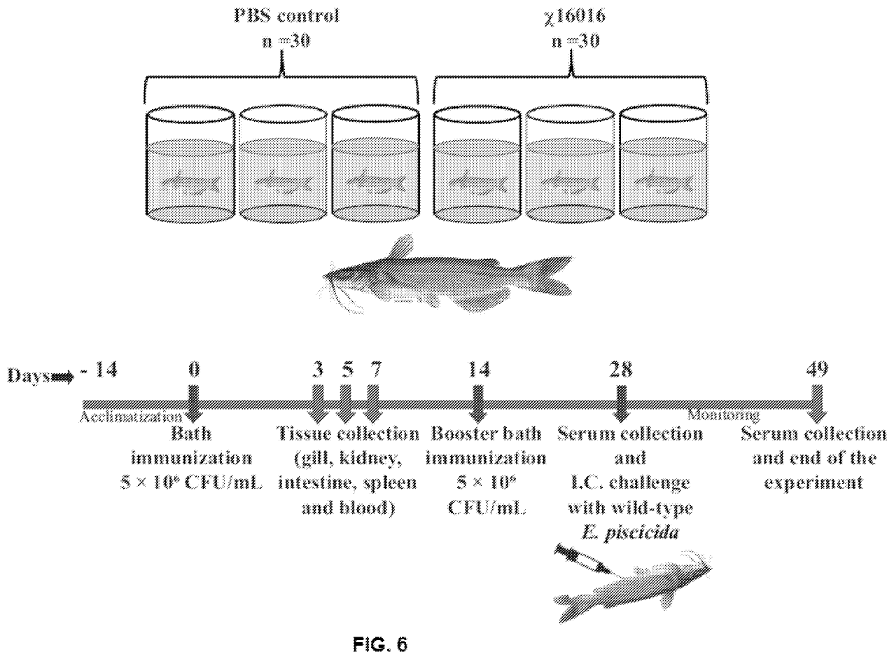

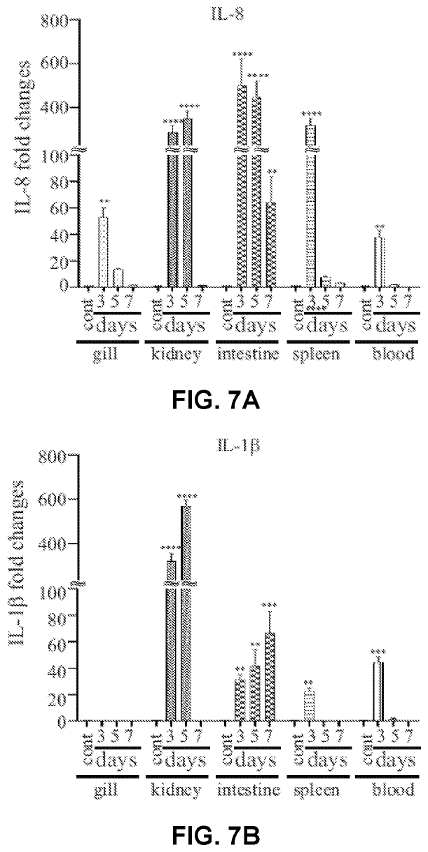

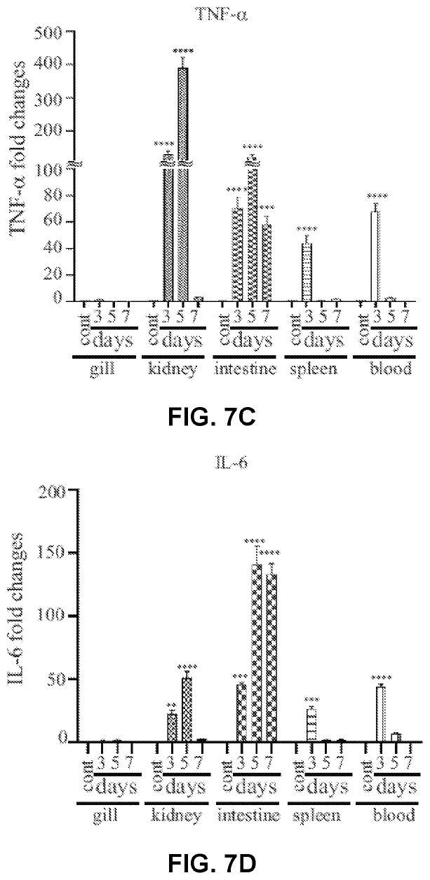

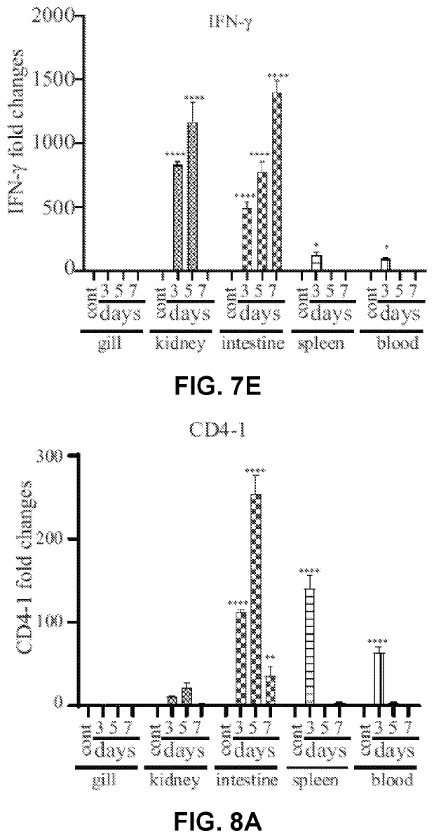

Changes in the expression patterns of il-8, il-1β, TNF-α, il-6, and ifn-γ genes in χ16016-vaccinated and control catfish. The vaccine strain χ16016 has a biological containment system, intended to cause programmed bacterial cell lysis upon invasion into host tissues (in an arabinose-free environment). Its potential as a vaccine candidate to induce innate and adaptive immunity was investigated in catfish fingerlings. The summary diagram of the experimental setup and sampling outline are shown in FIG. 6. The relative expression levels of cytokine genes il-8, il-1β, tnf-α, il-6 and ifn-γ were assessed in the vaccinated and control catfish gills, kidneys, intestines, spleens and blood. The expression of il-8 rapidly increased in all immunized fish tissues at days 3 and 5 compared to control fish tissues. The il-8 gene was expressed to a higher degree in the intestine compared to other tissues (FIG. 7A). Significant induction of il-1β was noted in all the tested tissues except for the gills. Highest expression in the kidneys was detected at day 5 (˜600-fold), highest expression in the intestines detected at day 7 (˜70-fold), and highest expression in the spleens and blood occurred on day 3 (˜30- and 40-fold) (FIG. 7B). tnf-α was expressed to a higher degree in the kidneys, intestines, spleens, and blood of immunized fish compared to control fish. At day 5, highest expression was in the kidneys (˜400-fold) and intestines (˜100-fold) whereas, at day 3, elevated expression was noticed in the spleens (˜50-fold) and blood (˜70-fold) (FIG. 7C). Increased expression of the il-6 gene was detected in immunized fish intestines at day 3 (˜50-fold), 5 (˜160-fold) and 7 (˜150-fold). Significant expression of il-6 occurred in the spleens (˜25-fold) and blood (˜50-fold) only on day 3. In the kidneys, it was upregulated on both day 3 (˜40-fold) and 5 (˜55-fold) (FIG. 70). ifn-γ showed very high expression levels in the intestines and kidneys. In the intestines, ifn-γ expression increased progressively on day 3 (˜500-fold), 5 (˜800-fold) and 7 (˜1500-fold). In the kidneys, expression was highest on day 5 (˜1300-fold), followed by day 3 (˜800-fold). In the spleens and blood, ifn-γ expression increased only on day 3 (FIG. 7E).

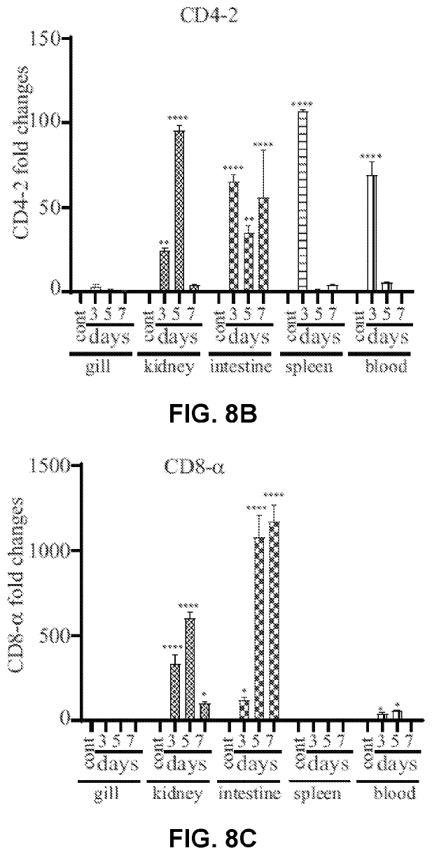

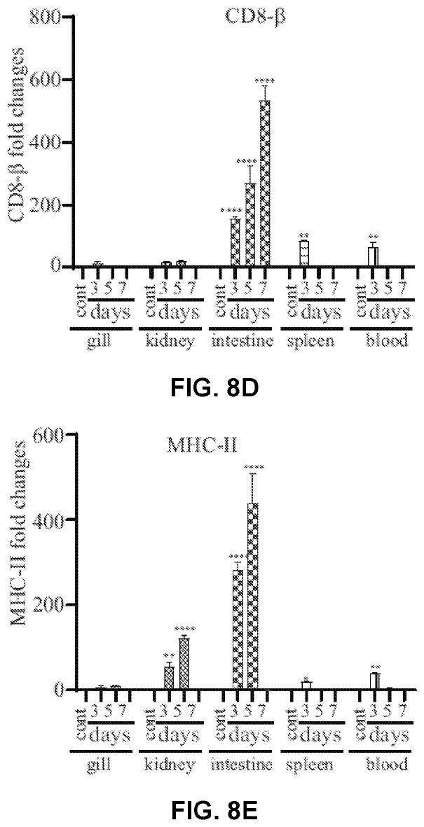

Changes in the expression patterns of the T cells-related genes in χ16016 vaccinated and control catfish. The major histocompatibility complex (MHC) molecules play an important role in presenting peptide antigens derived from pathogens on the cell surface for recognition by the appropriate T cells in the acquired immune system. The levels of mRNA expression of the mhc-II gene and T cell-specific genes (cd4-1, cd4-2, cd8-α and cd8-β) were evaluated; these are essential in driving adaptive immune responses upon vaccination. There was increased expression of both cd4-1 and cd4-2 in the kidneys and the intestines at all the tested time points. However, enhanced expression in the spleens and blood was only detected at day 3 (FIGS. 8A-8B). Both cd8-α and cd8-β were highly expressed in the intestines at days 3, 5 and 7, and expression increased continuously from day 3 to day 5 to day 7 (FIGS. 8C-8D). There was increased expression of mhc-II in the intestines and kidneys at days 3 and 5 (FIG. 8E).

Analysis of E. piscicida anti-LPS antibody responses in control and immunized catfish.

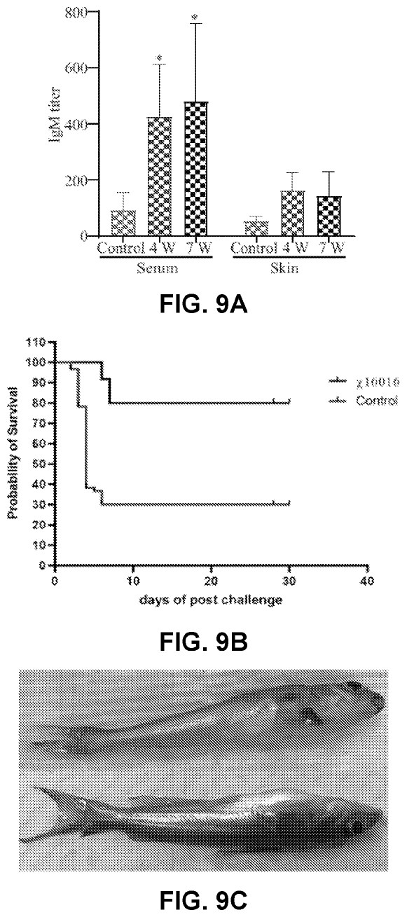

Serum and mucosal immunoglobulin M (IgM) responses to E. piscicida lipopolysaccharide (LPS) were measured by ELISA at 4 weeks (4 w) post-vaccination with χ16016 and 3 weeks after J118 challenge (7 w) of immunized fish. A significant increase of IgM titer was detected in the serum of bath-immunized fish at weeks 4 and 7 when compared to unimmunized control fish. Mucosal (skin) IgM titer steadily increased at weeks 4 and 7 in the immunized group (FIG. 9A).

Survival rate of vaccinated fish after wild-type E. piscicida challenge. To study the protective immunity induced by the vaccine strain χ16016, vaccinated catfish were challenged with the wild-type virulent E. piscicida strain J118. These results indicated that χ16016 confers a greater survival rate (80%) against a lethal dose challenge of virulent E. piscicida in comparison to that of the control unvaccinated fish (30%) over a period of 4 weeks post-challenge (FIG. 9B).

The clinical observation of catfish after challenge with E. piscicida. The majority of the mortalities occurred within 8 days post-challenge. Dead fish showed typical symptoms of edwardsiellosis, including hemorrhagic ulcers on the body surface and along the fins (FIG. 9C).

### DISCUSSION

The channel catfish (Ictalurus punctatus) is the top species produced in the US aquaculture industry, contributing about 75% of the finfish aquaculture volume with a 35% value share (FAO, 2020). Edwardsiella piscicida is a gram-negative intracellular pathogen responsible for significant losses in catfish aquaculture. Vaccination remains the most effective approach for prevention and control of infectious diseases in aquaculture. A safe and highly efficacious vaccine is of utmost importance to avoid economic losses and improve the food safety. An ideal vaccine for global fish aquaculture should be antibiotic-sensitive, highly immunogenic, environmentally safe, low cost, needle-free and cause minimal stress during application. Live vaccines possess potent adjuvant properties to elicit a high degree of immunogenicity, given that they are able to mimic natural infection and can be delivered by bath immersion or orally. Live attenuated vaccines containing a defined deletion mutation can persist in the vaccinated animal and have the potential to be released into the environment. This may lead to unintentional immunization of other species and may be problematic if these genetically modified organisms are able to persist in the environment. To address this potential risk, the employment of biological containment systems is required. Herein is disclosed a novel strategy to construct a biological containment system that is designed to cause programmed bacterial cell lysis with no potential for survival, thus preventing the spread of the vaccine strain into the environment. The recombinant attenuated Edwardsiella vaccine (RAEV) is the first live attenuated vaccine designed for use in the aquaculture industry that displays this biological containment property. The murA gene encodes enzymes involved in the first steps of peptidoglycan biosynthesis within the bacterial cytoplasm. Since the product of the murA gene is a phosphorylated sugar that cannot be taken up by bacteria, murA deletion is lethal. The murA encoded protein in fish pathogen E. piscicida is highly conserved and has a 3D structure similar to that of the well-studied human intracellular bacterial pathogen Salmonella sp. (FIGS. 1A-1B). In this study, a conditional-lethal murA mutation was created by replacing the wild type murA promoter in E. piscicida with a tightly regulated araC P_{araBAD} promoter. This makes it so that the expression of the murA gene is dependent on arabinose, which is provided during growth in vitro (FIGS. 2G-2H). In the absence of arabinose, such as in fish tissues, muramic acid is not synthesized, peptidoglycan is not produced, and lysis of the bacterium occurs. The lysis strain χ16016 has the ability to colonize fish tissues (FIGS. 5A-5B), a requirement for the induction of a strong immune response, but eventually dies due to lysis, demonstrating the biological containment feature (FIG. 5C). The lysis strain has a similar capacity to attach and internalize into the fish cell line as the parental strain. However, its intracellular replication rate was slower compared to the wild-type, possibly due to the dilution of pre-formed MurA enzyme with each round of cell division and the absence of arabinose inside the cell (FIGS. 3A-3C). Vaccines with regulated lysis are excellent adjuvants; they efficiently stimulate innate immunity as the vaccine follows the natural infection process and eventually lyses to deliver ligands and stimulate pattern recognition receptors (PRRs). The lysis strain stimulates cell surface-expressed TLRs, such as TLR4 and TLR5, which are involved in the primary encounter of the pathogen with the host. RAEVs deliver peptidoglycan subunits and nucleic acids upon lysis to activate intracellular PRRs NOD1, NOD2 TLR8 and TLR9. These results demonstrate that lysis strain χ16016 stimulates TLRs and NLRs, leading to the activation of transcription factor NF-κB (FIGS. 4A-4F), important for the innate response and for the establishment of the adaptive immune response. Th1-type cytokines IL-1β, IFN-γ, and TNF-α are important in controlling intracellular infections. IL-6 is involved in B cell maturation and macrophage differentiation, and is produced by antigen presenting cells (APC) such as macrophages, dendritic cells, and B cells. Recombinant IL-6 has been used as an adjuvant in fish to induce cellular and humoral immunity and immune protection against E. tarda infection. IL-8 is an important chemokine that plays a key role in the recruitment of T cells and nonspecific inflammatory cells to the site of infection by activating neutrophils. Induced expression of il-8, il-1β, tnf-α, il-6 and ifn-γ genes was observed in the gills, kidney, intestine, spleen and blood of χ16016 immunized catfish. In a previous study, it has been shown that RAEVs are able to efficiently stimulate these cytokines. These cytokine inductions may lead to the establishment of humoral immunity. In the adaptive immune response, major histocompatibility complex class II (MHC-II) molecules play critical roles in antigen presentation. Significant increased expression of cd4, cd8 and mhc-II was observed in the different organs of vaccinated catfish. These findings indicate the initiation of an adaptive immune response to E. piscicida. In teleosts, IgM is a primary antibody in humoral immunity. Since catfish do not have an IgT antibody isotype, IgM serves a vital function in both mucosal and systemic immune responses. The vaccine specific IgM titer is an important adaptive immune parameter to study in immunized fish. It was observed that the vaccine specific IgM antibody levels increased in both the serum and skin of vaccinated fish compared to the control fish. The immune protection mediated by the RAEV lysis vaccine strain χ16016 was evaluated against E. piscicida infection and found that the survival rate was higher (80%) in the immunized group than the control group (30%).

In summary, herein is described the design and construction of a recombinant attenuated Edwardsiella vaccine (RAEV) with a regulated lysis phenotype. It creates a biological containment system that leads to programmed bacterial cell lysis, thus preventing persistence in vivo and the spread of the vaccine strain into the environment. This lysis strain possesses potent adjuvant properties to stimulate the immune system and confers significant protection to catfish against wild-type E. piscicida infection. This system can be added to E. piscicida strain χ16022 to use as an antigen or DNA delivery vector for the aquaculture industry.

### Example 2: Codon Optimization and Protective Antigen Selection

Synthesis of protective antigens by a pathogen such as A. hydrophilia use different codons in the genes encoding them that are best for gene expression in A. hydrophlia. Thus, to enable improved expression of these genes when inserted in expression plasmids in the E. piscicida vaccine vector strains it is desirable to change the encoding codons to those that are used by E. piscicida for highly expressed genes. This first requires determining which codons are used most often in highly expressed genes. Then one can use these in resynthesizing the genes encoding protective antigens with codons that will ensure high-level synthesis of those antigens in the vaccine vector strain.

### Materials and Methods

Bacterial strains, media and bacterial growth. All RAEV strains are derived from derived from J118, an R plasmid-cured derivative of the highly virulent E. piscicida EIB202, which has been sequenced. J118, which has now lost all antibiotic-resistance genes for resistance to streptomycin, tetracycline, chloramphenicol and sulfonamide and is sensitive to all antibiotics, has the same high virulence as its parent EIB202. LB broth and agar and Purple broth (PB) (Difco), which is devoid of arabinose (Ara) phenotypic analyses and plating. Bacterial growth is monitored spectrophotometrically and by plating for colony counts.

Protective antigen selection. Selection of protective antigens to be encoded on regulated lysis plasmid vectors for synthesis and delivery by RAEV strains or to be encoded on DNA vaccine vectors for expression in the vaccinated animal host are based on evidence in the published literature or deduced based on data for contribution elicitation of protective immune responses or based on bioinformatic searches using known information about other pathogens.

An extensive literature search has resulted in a list of A. hydrophila protective antigens. Initially, nine proteins of A. hydrophila were selected as potential protective antigens. The degrees of immunogenicity of the antigens were identified by locating the B-cell and T-cell epitopes in the antigen by BepiPred and NetCTL servers respectively. The subcellular location of the individual proteins was predicted by the CELLO and pSORTb predictors. Commonly, protective B & T-cell protein antigens are located in the outer membrane and extracellular environment, hence these predicted subcellular locations were targets for selection. Conservation of antigens among Aeromonas species was done by NCBI BLAST searches. Antigens that have more than 90% identity and 95% query coverage among Aeromonas species are included. Conserved antigens were included in this study to increase the probability of success, due to the ability to elicit protection across different Aeromonas strains. All antigens except AGM42919 were chosen based on function, location, conservation and immune response. AGM42919 was chosen based on function, location and immune response.

RAEV strain characterization. RAEV constructs are evaluated in comparison with vector control strains for stability of plasmid maintenance, integrity and protein synthesis ability when RAEVs are grown in the presence of arabinose and DAP and with and without IPTG for 50 generations. The IPTG dependence of protein synthesis to overcome the LacI repression of the Ptrc promoter is also verified.

Activation of TLRs and NLRs by RAEV strain. It was hypothesized that RAEVs with the regulated-delayed lysis attribute could be used as an adjuvant to activate innate immunity by delivering highly conserved microbial structures, including lipopolysaccharide, flagella, CpG DNA, lipoprotein, and peptidoglycan (PGN) fragments (diaminopimelic acid (DAP) and muramyl dipeptide (MDP)). The detection of conserved microbial motifs relies on the two major classes of PRRs, including toll-like receptors (TLRs) and NOD-like receptors (NLRs). To investigate the activation of TLRs and NLRs by χ16016, HEK-Blue cells expressing mouse TLR4, TLR5, TLR8, TLR9, NOD1 and NOD2 (all from InvivoGen) were used with a NFkB-inducible secreted embryonic alkaline phosphatase (SEAP) reporter gene. The protocol as described previously was followed. In brief, cells were maintained in DMEM growth medium at 37° C. in 5% CO₂. Cells were cultured in the presence of selective antibiotics, as recommended by the manufacturer, and passaged twice a week at 80-90% confluency. Bacterial cells were grown in LB broth with arabinose (χ16016) or without arabinose (J118) to an OD₆₀₀ of 0.8. Bacterial cultures were sedimented at room temperature and resuspended in tissue culture medium. Approximately 1×10⁵ HEK-Blue cells were suspended in 200 μL HEK-Blue™ Detection medium (Invivogen) and were mixed with bacterial cells at a MOI of 1 in 96-well cell culture plates (Corning). Uninfected HEK-Blue cells with HEK-Blue™ detection medium was used as a control. After 3, 6, 12 and 24 h of incubation at 37° C. in 5% CO₂, SEAP activity was determined at 655 nm according to the manufacturer's recommendations (InvivoGen). All samples were measured in triplicate. TLR and NLR stimulation were expressed relative to the level of SEAP activity of uninfected control cells.

Determination of Lethal Dose 50 (LD₅₀) in Fish by Bath Immersion and I.C. Injection. To determine the LD₅₀ of RAEV, ten-fold serial dilutions of E. piscicida were performed, fresh cultures were made in sterile BSG and the concentration of bacteria was determined using the spread-plate method. Fish were i.e. injected with a dose of 10-100 μL of BSG containing different concentrations of CFU per fish. Mortality was documented daily over a 21-day period, and the Reed and Muench method was used to calculate the LD₅₀ values. For the bath immersion, fish were immersed in tank water containing specific concentrations of bacteria ranging from 1×10³, 1×10⁴, 1×10⁶ and 1×10⁸ CFU mL⁻¹. After 2 h, the fish were removed from the solution and placed into their respective original tanks.

Colonization and Lysis of χ16016 in Catfish Tissues. Catfish fingerlings were vaccinated with χ16016 via bath immersion for 2 hours with 1×10⁶ CFU/mL. Kidneys and intestines were collected from fish at days 1, 2, 3 and 4 post-vaccination (five fish at each time point). The tissues were homogenized in 200 μL of BSG and a 10-fold serial dilution of each sample was plated on LB agar plates containing colistin sulfate (Col) (12.5 μg/mL) and 0.2% arabinose. The plates were incubated at 30° C. for 48 h and the colonies were counted. The data consisted of a combination of three independent assays. For the lysis study, catfish fingerlings were i.e. injected with χ16016 or J118 with a dose of 1×10³ CFU/fish. Kidneys were collected from fish every other day up to 16 days post-vaccination (five fish at each time point). The tissues were homogenized in 200 μL of BSG and a 10-fold serial dilution of each sample was plated on LB agar plates containing colistin sulfate (Col) (12.5 μg/mL) and 0.2% arabinose. The data consisted of a combination of three independent assays. Differences between two groups were analyzed by two-way ANOVA, where asterisks (*) indicate a significant difference (**P<0.01, ****P<0.0001).

Evaluation of anti-E. piscicida IgM in Catfish Serum and Skin Mucus by Enzyme-Linked Immunosorbent Assay (ELISA). To assay antibodies in catfish serum and skin mucus, ELISA was performed using E. piscicida LPS following the protocol described previously. Polystyrene 96-well flat-bottom microtiter plates (Dynatech Laboratories Inc., Chantilly, Va.) were coated with 100 μL (in each well) of E. piscicida LPS (100 ng/well) in sodium carbonate-bicarbonate coating buffer (pH 9.6). The coated plates were incubated at 4° C. overnight. Free binding sites were blocked by adding 300 μL of 5% bovine serum albumin (BSA) in each well and plates were incubated for 1 h at room temperature. After washing, 100-μL of diluted catfish serum/mucus samples from control and vaccinated fish were added to individual wells in duplicate. 100 μL of sterile PBS was added to the blank control wells and incubated for 2 h at 37° C. After washing, mouse anti-catfish IgM monoclonal antibody (Aquatic Diagnostics Ltd, UK) was diluted 1:100 in PBS and 100 μL was added to each well and incubated for 1 h at room temperature. Biotinylated goat anti-mouse IgG (Southern Biotechnology Associates, Birmingham, AL) was diluted with 1% BSA and 100 μL was added to each well and incubated for 1 h at room temperature. After incubation of wells with a streptavidin-alkaline phosphatase conjugate (Southern Biotechnology) for 1 h at 37° C., p-nitrophenyl phosphate (PNPP, Thermo Fisher Scientific) was added for color development. The optical density (OD) units were read at 405 nm using an automated ELISA plate reader (model EL311SX; Biotek, Winooski, VT).

### Codon Optimization

Determination of Frequency of Codon Use for Amino Acids of E. piscicida. In all prokaryotic and eukaryotic genomes codon usage biases are found, and preferred codons are more frequently used in highly expressed genes. Codon optimization has long since been used to enhance protein expression for heterologous gene expression. In addition, the fact that highly expressed proteins are mostly encoded by genes with mostly optimal codons. An extensive literature search was done and 118 of highly expressed genes in E. coli and Salmonella were listed. These gene sequences were retrieved from NCBI database of Edwardsiella piscicida (EIB202) genome sequence. Using these highly expressed gene sequences of E. piscicida, the frequency of codon use for amino acids was determined with the help of online codon usage calculator and presented in Table 6. Such codon frequency uses for highly expressed genes are in contrast to codon frequency uses in all genes in the E. piscicida genome (Table 6). This information was used to codon-optimize different genes of A. hydrophila and tilapia lake virus (TiLV) for high-level expression in the RAEV vaccine strains. In most instances for most amino acids the most frequently used codon for that amino acid was used although for some amino acids two or even all codons for that amino acid had nearly the same high frequency of use. As another important attribute to achieve high levels of antigen synthesis, it was important to have the GC content of the entire DNA sequence to be closely similar or nearly identical to the GC content of E. piscicida DNA which is 60%. For this reason, achieving the desired 60% GC content also influenced the codons selected for use.

TABLE 6
E. piscicida codon usages

| 3- | Single | | Number | | /1000 | | Fraction | |
|---|---|---|---|---|---|---|---|---|
| letter | letter | | Highly | Average | Highly | Average | Highly | Average |
| code | code | Codon | Expressed | all genes | Expressed | all genes | Expressed | all genes |
| Ala | A | GCG | 1126.00 | 13704.00 | 37.23 | 41.11 | 0.34 | 0.36 |
| Ala | A | GCA | 319.00 | 6617.00 | 10.55 | 19.85 | 0.10 | 0.17 |
| Ala | A | GCT | 409.00 | 7986.00 | 13.52 | 23.96 | 0.12 | 0.21 |
| Ala | A | GCC | 1467.00 | 9561.00 | 48.50 | 28.68 | 0.44 | 0.25 |
| Cys | C | TGT | 72.00 | 3640.00 | 2.38 | 10.92 | 0.37 | 0.32 |
| Cy | C | TGC | 124.00 | 7742.00 | 4.10 | 23.23 | 0.63 | 0.68 |
| Asp | D | GAT | 836.00 | 6892.00 | 27.64 | 20.68 | 0.50 | 0.61 |
| Asp | D | GAC | 824.00 | 4378.00 | 27.24 | 13.13 | 0.50 | 0.39 |
| Glu | E | GAG | 747.00 | 4489.00 | 24.70 | 13.47 | 0.56 | 0.54 |
| Glu | E | GAA | 590.00 | 3766.00 | 19.51 | 11.30 | 0.44 | 0.46 |
| Phe | F | TTT | 446.00 | 5054.00 | 14.75 | 15.16 | 0.45 | 0.58 |
| Phe | F | TTC | 550.00 | 3652.00 | 18.18 | 10.96 | 0.55 | 0.42 |
| Gly | G | GGG | 398.00 | 6710.00 | 13.16 | 20.13 | 0.16 | 0.24 |
| Gly | G | GGA | 162.00 | 4627.00 | 5.36 | 13.88 | 0.07 | 0.16 |
| Gly | G | GGT | 601.00 | 5873.00 | 19.87 | 17.62 | 0.24 | 0.21 |
| Gly | G | GGC | 1323.00 | 10897.00 | 43.74 | 32.69 | 0.53 | 0.39 |
| His | H | CAT | 196.00 | 4326.00 | 6.48 | 12.98 | 0.37 | 0.56 |
| His | H | CAC | 339.00 | 3349.00 | 11.21 | 10.05 | 0.63 | 0.44 |
| Ile | I | ATA | 70.0 | 3571.00 | 2.31 | 10.71 | 0.05 | 0.27 |
| Ile | I | ATT | 342.00 | 3381.00 | 11.31 | 10.14 | 0.24 | 0.26 |
| Ile | I | ATC | 1008.00 | 6160.00 | 33.33 | 18.48 | 0.71 | 0.47 |
| Lys | K | AAG | 736.00 | 3689.00 | 24.33 | 11.07 | 0.51 | 0.50 |
| Lys | K | AAA | 703.00 | 3723.00 | 23.24 | 11.17 | 0.49 | 0.50 |
| Leu | L | TTG | 118.00 | 3976.00 | 3.90 | 11.93 | 0.04 | 0.16 |
| Leu | L | TTA | 85.00 | 2828.00 | 2.81 | 8.48 | 0.03 | 0.12 |
| Leu | L | CTG | 2159.00 | 9358.00 | 71.38 | 28.07 | 0.76 | 0.38 |
| Leu | L | CTA | 93.00 | 1875.00 | 3.07 | 5.63 | 0.03 | 0.08 |
| Leu | L | CTT | 109.00 | 3027.00 | 3.60 | 9.08 | 0.04 | 0.12 |
| Leu | L | CTC | 267.00 | 3354.00 | 8.83 | 10.06 | 0.09 | 0.14 |
| Met | M | ATG | 709.00 | 5323.00 | 23.44 | 15.97 | 1.00 | 1.00 |
| Asn | N | AAT | 400.00 | 2967.00 | 13.23 | 8.90 | 0.29 | 0.49 |
| Asn | N | AAC | 975.00 | 3115.00 | 32.24 | 9.35 | 0.71 | 0.51 |
| Pro | P | CCG | 816.00 | 8950.00 | 26.98 | 26.85 | 0.64 | 0.38 |
| Pro | P | CCA | 73.00 | 5582.00 | 2.41 | 16.75 | 0.06 | 0.24 |
| Pro | P | CCT | 105.00 | 4455.00 | 3.47 | 13.37 | 0.08 | 0.19 |
| Pro | P | CCC | 290.00 | 4373.00 | 9.59 | 13.12 | 0.23 | 0.19 |
| Gln | Q | CAG | 1452.00 | 8072.00 | 48.01 | 24.22 | 0.89 | 0.74 |
| Gln | Q | CAA | 186.00 | 2886.00 | 6.15 | 8.66 | 0.11 | 0.26 |
| Arg | R | AGG | 25.00 | 5474.00 | 0.83 | 16.42 | 0.01 | 0.13 |
| Arg | R | AGA | 25.00 | 3863.00 | 0.83 | 11.59 | 0.01 | 0.09 |
| Arg | R | CGG | 219.00 | 9668.00 | 7.24 | 29.00 | 0.11 | 0.23 |
| Arg | R | CGA | 69.00 | 6075.00 | 2.28 | 18.23 | 0.04 | 0.14 |
| Arg | R | CGT | 626.00 | 5749.00 | 20.70 | 17.25 | 0.33 | 0.13 |
| Arg | R | CGC | 962.00 | 12139.00 | 31.81 | 36.42 | 0.50 | 0.28 |
| Ser | S | AGT | 109.00 | 2617.00 | 3.60 | 7.85 | 0.06 | 0.09 |
| Ser | S | AGC | 853.00 | 7575.00 | 28.20 | 22.73 | 0.44 | 0.26 |
| Ser | S | TCG | 259.00 | 6241.00 | 8.56 | 18.72 | 0.13 | 0.22 |
| Ser | S | TCA | 93.00 | 4476.00 | 3.07 | 13.43 | 0.05 | 0.16 |
| Ser | S | TCT | 249.00 | 3705.00 | 8.23 | 11.12 | 0.13 | 0.13 |
| Ser | S | TCC | 366.00 | 4084.00 | 12.10 | 12.25 | 0.19 | 0.14 |
| Thr | T | ACG | 386.00 | 5076.00 | 12.76 | 15.23 | 0.24 | 0.35 |
| Thr | T | ACA | 56.00 | 2589.00 | 1.85 | 7.77 | 0.03 | 0.18 |
| Thr | T | ACT | 165.00 | 2253.00 | 5.46 | 6.76 | 0.10 | 0.16 |
| Thr | T | ACC | 1006.00 | 4481.00 | 33.26 | 13.44 | 0.62 | 0.31 |
| Val | V | GTG | 704.00 | 5444.00 | 23.28 | 16.33 | 0.33 | 0.31 |
| Val | V | GTA | 327.00 | 3303.00 | 10.81 | 9.91 | 0.15 | 0.19 |
| Val | V | GTT | 518.00 | 4009.00 | 17.13 | 12.03 | 0.24 | 0.23 |
| Val | V | GTC | 574.00 | 4725.00 | 18.98 | 14.18 | 0.27 | 0.27 |
| Trp | W | TGG | 372.00 | 8066.00 | 12.30 | 24.20 | 1.00 | 1.00 |
| Tyr | Y | TAT | 484.00 | 3872.00 | 16.00 | 11.62 | 0.46 | 0.57 |
| Tyr | Y | TAC | 571.00 | 2881.00 | 18.88 | 8.64 | 0.54 | 0.43 |
| End | * | TGA | 3.00 | 6081.00 | 0.27 | 18.24 | 0.17 | 0.55 |
| End | * | TAG | 0.00 | 2151.00 | 0.00 | 6.45 | 0.00 | 0.19 |
| End | * | TAA | 15.00 | 2808.00 | 1.35 | 8.42 | 0.83 | 0.25 |

An extensive literature search has resulted in a list of A. hydrophila protective antigens. Initially, nine proteins of A. hydrophila were selected as potential protective antigens. The degrees of immunogenicity of the antigens were identified by locating the B-cell and T-cell epitopes in the antigen by BepiPred and NetCTL servers respectively. The subcellular location of the individual proteins was predicted by the CELLO and pSORTb predictors. Commonly, protective B- and T-cell protein antigens are located in the outer membrane and extracellular environment, hence these predicted subcellular locations were targets for selection. Conservation of antigens among Aeromonas species was done by NCBI BLAST searches. Antigens that have more than 90% identity and 95% query coverage among Aeromonas species are included. In Table 7, the percentage of identity of antigens with A. salmonicida are represented. Conserved antigens were included in this study to increase the probability of success, due to the ability to elicit protection across different Aeromonas strains. All antigens except AGM42919 were chosen based on function, location, conservation and immune response. AGM42919 was chosen based on function, location and immune response.

TABLE 7
Rationale for Antigen Selection

| | | | Conservation | |
|---|---|---|---|---|
| | | | Identity % with | |
| Protein ID | Function | Location | A. salmonicida | Immune response |
| CCO02501 | transmembrane portions | Outer | 91 | Induce Th1/Th2 |
| | of porins | membrane | | immune response |
| AGM43135 | Outer membrane integral | Outer | 93 | 98.59% RPS In |
| | membrane protein | membrane | | catfish |
| AGM42919 | TonB-dependent receptor | Outer | 24 | 95.59% RPS in |
| | | membrane | | catfish |
| A0KFG8 | Ferrichrome-iron receptor | Outer | 94 | 70% RPS in |
| | | membrane | | zebrafish |
| A0KGW8 | TonB-dependent | Outer | 93 | 61.7% RPS in |
| | hemoglobin/transferrin/ | membrane | | zebrafish |
| | lactoferrin family receptor | | | |
| A0KQ46 | ligand-gated channel | Outer | 96 | 54.9% in |
| | protein | membrane | | zebrafish |
| A0KQZ1 | TonB-dependent | Outer | 98 | 60.7% in RPS in |
| | siderophore receptor | membrane | | zebrafish |
| A0KIU8 | TonB-dependent | Outer | 91 | 68.6% RPS in |
| | hemoglobin/transferrin/ | membrane | | zebrafish |
| | lactoferrin family receptor | | | |
| AGH12866 | Outer membrane porin | Outer | 91 | 83.33% RPS |
| | OmpC | membrane | | European eels |

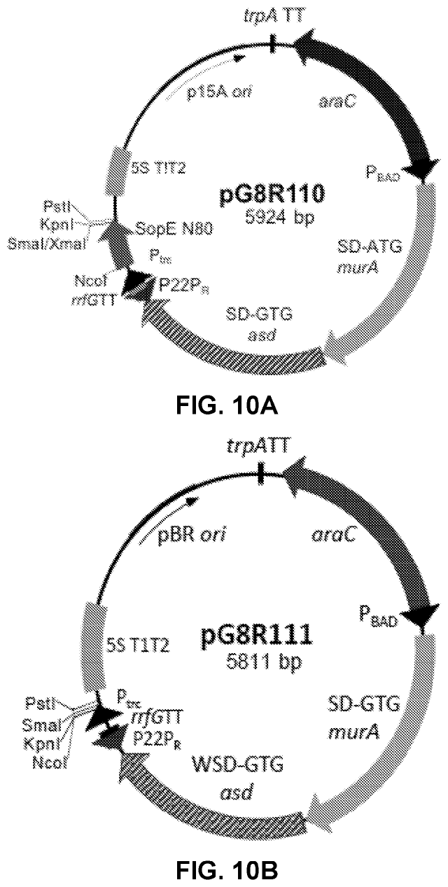

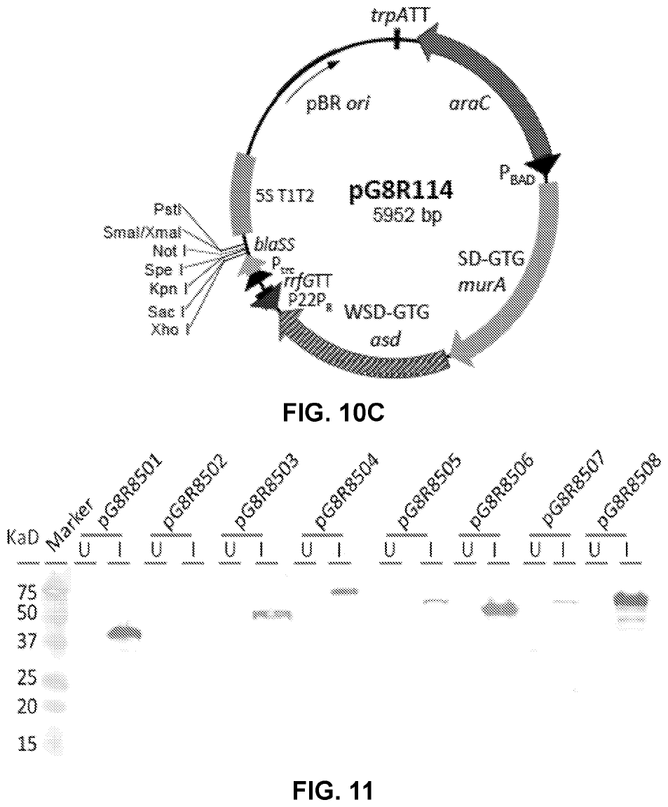

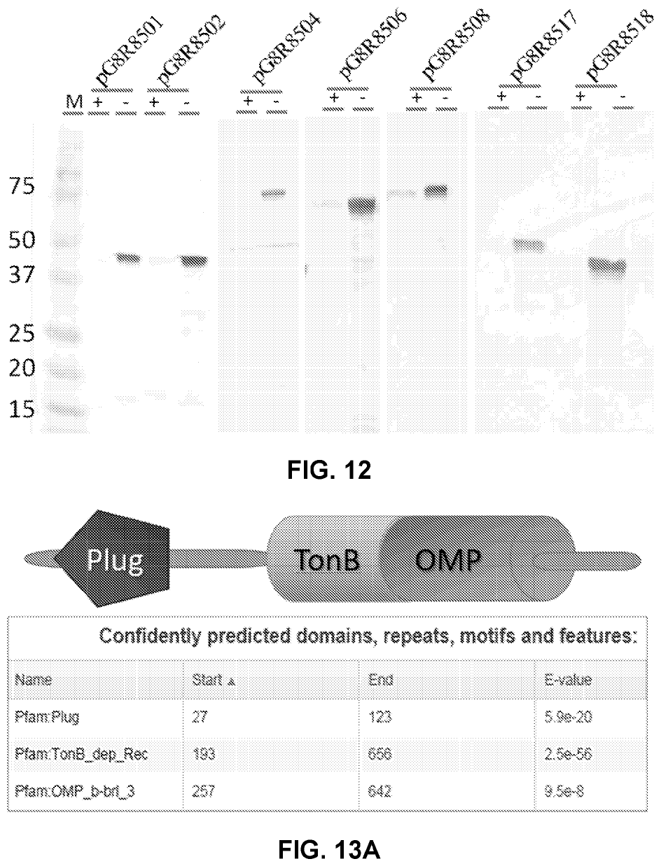

Generation of a regulated delayed lysis plasmid encoding different protective A. hydrophila antigens with expression of coding sequences regulated by LacI regulated promoters secreted by type 2 secretion systems. To achieve the optimal levels of antigen synthesized, pG8R114 (FIGS. 10A-10C) vector was used with the fusion of antigens to the bla SS (T2SS) leads to the delivery of antigens to the periplasm, resulting in an increased production of outer membrane vesicles (OMVs) that enhance immunogenicity and antibody production against delivered antigens. In addition, use of Type 2 secretion for protective antigen delivery also leads to protective antigen released into the supernatant fluid surrounding cells to also enhance the level of induced immune responses. Eleven constructs have already been generated to synthesize different putative protective A. hydrophila antigens (Table 8). Codon-optimized sequences of A. hydrophila antigen genes with sequence encoding C-terminal His-tags were PCR-amplified and cloned into pG8R114 and the resultant plasmids were named as pG8R8501-pG8R8508, pG8R8510, pG8R8517 and pG8R8518 (Table 8). They were electroporated into E. coli χ6212(pYA232) cells. In E. coli, antigen syntheses were analyzed in IPTG-induced (I) and uninduced (U) cells by western blotting with anti-His-tag antibody (FIG. 11). pYA232 has a pSC101 replicon compatible with the pBR on present in both plasmid constructs and possesses the lacI^{Q} gene so that expression of genes under the control of the Asd+vector P_{trc} promoter is repressed. This requires addition of inducer IPTG to relieve LacI repression and permit transcription of encoding genes in recombinant Asd⁺ vectors. These plasmids were also incorporated into E. piscicida vaccine strains χ16034 or χ16035 individually. In E. piscicida, A. hydrophila antigen synthesis was analyzed in the presence (+) or absence (−) of arabinose (FIG. 12). Because the strains χ16034 or χ16035 display the regulated delayed antigen synthesis (RDAS) system. In the presence of arabinose LacI is produced, which binds to P_{trc}, blocking antigen synthesis. Therefore, in the presence of LacI, all strains showed reduced A. hydrophila antigen synthesis which was detected by western blotting with anti-his-tag antibody (FIG. 12). The presence of LacI in all strains reduced antigen expression in vitro, resulting in a faster growth rate. It was difficult to synthesize high concentrations of antigens in E. piscicida, but after adding RDAS technology to the disclosed vaccine system it became much easier to synthesize antigens. This system reduces the negative effects of antigen expression during in vitro growth, thereby improving the overall health of the vaccine strain, while allowing for maximum antigen expression in host tissues. This technology should be particularly useful for inducing immune responses to antigens that are toxic to the vaccine strain synthesizing them.

TABLE 8
List of A. hydrophila Antigens Synthesized in E. piscicida Vaccine Strain

| | | | Protein | Protein Synthesis | |
|---|---|---|---|---|---|
| | Protein | Parent | size in | E. coli | E. piscicida |
| Plasmid # | ID | plasmid | kDa | χ6212(pYA232) | vaccine strain |
| pG8R8501 | CCO02501 | pG8R114 | 42.7 | Detected by WB | Detected by |
| | | | | | WB |
| pG8R8502 | AGM43135 | pG8R114 | 42.1 | Detected by WB | Detected by |
| | | | | | WB |
| pG8R8503 | AGM42919 | pG8R114 | 80.6 | Detected by WB | Not tested |
| pG8R8504 | A0KFG8 | pG8R114 | 79.3 | Detected by WB | Detected by |
| | | | | | WB |
| pG8R8505 | A0KGW8 | pG8R114 | 81.2 | Detected by WB | Not tested |
| pG8R8506 | A0KQ46 | pG8R114 | 76.2 | Detected by WB | Detected by |
| | | | | | WB |
| pG8R8507 | A0KQZ1 | pG8R114 | 79.7 | Detected by WB | Not tested |
| pG8R8508 | A0KIU8 | pG8R114 | 81.1 | Detected by WB | Detected by |
| | | | | | WB |
| pG8R85010 | AGH12866 | pG8R114 | 41.2 | Detected by WB | Not tested |
| pG8R8517 | TonB | pG8R114 | 52.4 | Detected by WB | Detected by |
| | domain of | | | | WB |
| | A0KFG8 | | | | |
| pG8R8518 | OMP | pG8R114 | 43.3 | Detected by WB | Detected by |
| | domain of | | | | WB |
| | A0KFG8 | | | | |

Immune Protection of RAEV-Ah Strain χ16018(pG8R8517) Against Virulent Wild-Type A. hydrophila in Zebrafish Model

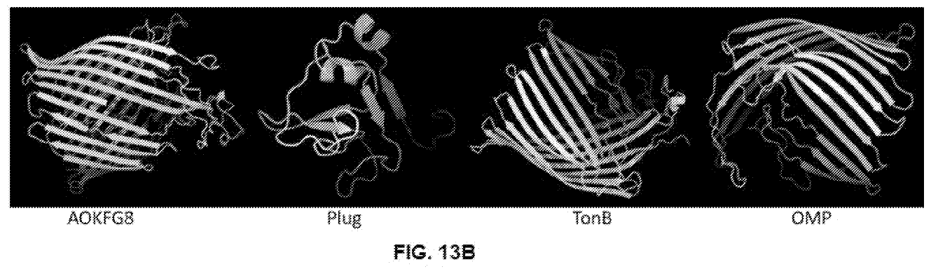

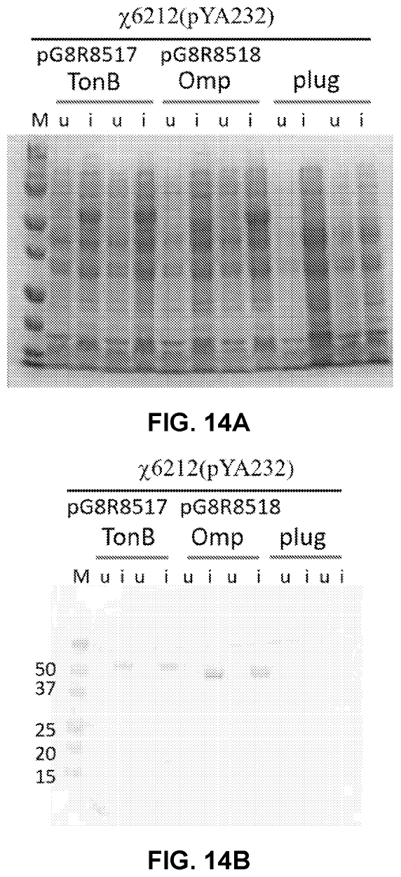

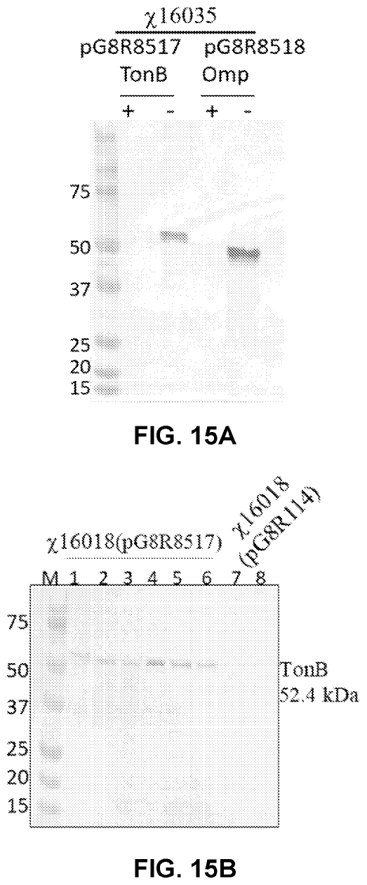

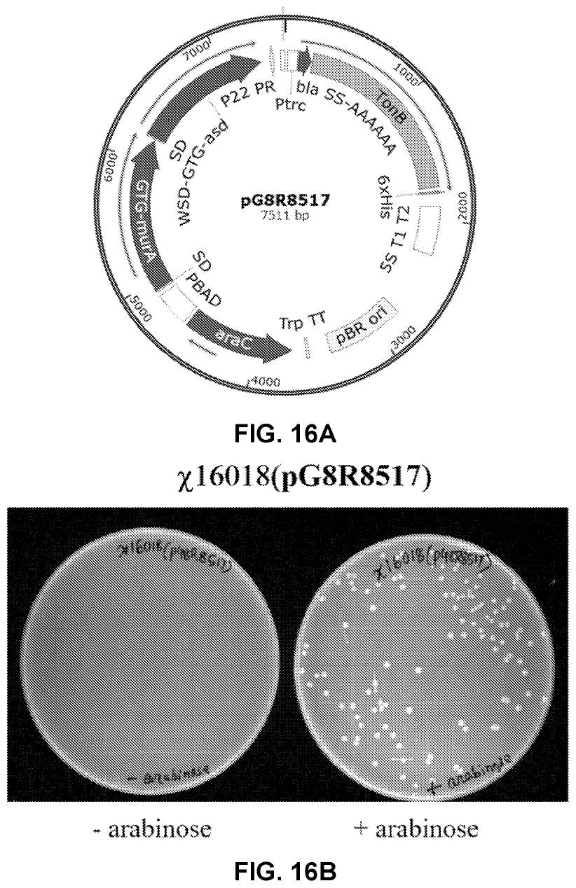

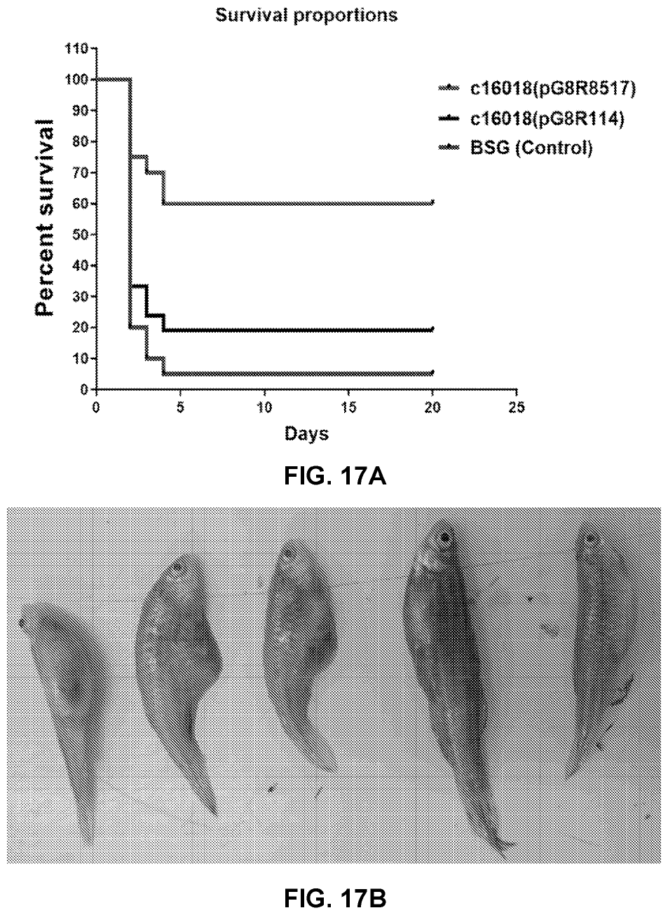

Different domains (Plug, TonB and OMP) of A. hydrophila antigen AOKFG8 were predicted by online SMART program (FIG. 13A) and their 3D structures were predicted by using the Phyre2 web portal (FIG. 13B). Codon-optimized sequences of Plug, TonB and OMP antigen gene segments with sequence encoding C-terminal His-tags were PCR-amplified and cloned into pG8R114 and the resultant plasmids were electroporated into E. coli χ6212(pYA232) cells. In E. coli, antigen syntheses were analyzed in IPTG-induced (I) and uninduced (U) cells by SDS PAGE (FIG. 14A) and western blotting with anti-His-tag antibody (FIG. 14B). The result reviled that, only TonB (pG8R8517) and OMP (pG8R8518) segments were synthesized whereas Plug domain was not detected either by SDS PAGE or by western blotting. Plasmid pG8R8517 and pG8R8518 were inserted into the E. piscicida strain χ160³⁵ and TonB and OMP synthesis were confirmed in absence arabinose (FIG. 15A). Plasmid pG8R8517 was inserted into the E. piscicida vaccine strain χ16018, synthesis of TonB was confirmed in six different colonies (FIG. 15B). Plasmid pG8R8517, which encodes the A. hydrophila antigen TonB (FIG. 16A) was introduced into lysis RAEV strain χ16018 with genotype ΔasdA10 ΔP_{fur170}::TT araC P_{araBAD} fur ΔP_{murA180}::TT araC P_{araBAD} murA. The resultant strain is χ16018(pG8R8517). The growth of this strain was dependent on arabinose (FIG. 16B). This feature of the lysis strains demonstrates complete biological containment. To investigate whether vaccination with χ16018(pG8R8517) producing TonB could induce protective immunity in zebrafish against A. hydrophila infection, fish were immunized by i.e. injection with 1×10⁴ CFU χ16018(pG8R8517)/fish and two control groups were injected with either χ16018(pG8R114) (vector control) or buffered saline containing 0.01% gelatin (BSG). At 4 weeks post-immunization, both immunized and control groups were i.e. challenged with 1.5×10⁴ CFU of virulent A. hydrophila/fish (20×LD₅₀). These results indicated that the zebrafish immunized with χ16018(pG8R8517) showed 60% higher survival compared to control groups (FIG. 17A). Fish that died after infection showed typical MAS disease symptoms (FIG. 17B).

### Example 3: E. piscicida Vaccines

Development of E. piscicida Vaccine Vector Strains

Herein are disclosed a series of E. piscicida mutant strains derived from J118, an R plasmid-cured derivative of the highly virulent E. piscicida E1B202, which has been sequenced. J118 is sensitive to all antibiotics. Suicide vector technologies were used with pRE112 to insert all the deletion and deletion-insertion mutations into J118 to generate the RAEV vaccine delivery vector strains χ16034 (ΔasdA10 ΔP_{fur170}::TT araC P_{araBAD} fur ΔP_{murA180}:TT araC P_{araBAD} murA ΔP_{crp68}::TT araC P_{araBAD} crp ΔrelA20::araC P_{araBAD} lacI TT) and χ16035 (ΔasdA 10ΔP_{fur170}::TT araC P_{araBAD} fur ΔP_{crp}::TT araC P_{araBAD} crp TT ΔrelA20::araC P_{araBAD} lacI TT).

### Regulated Delayed Lysis Phenotype

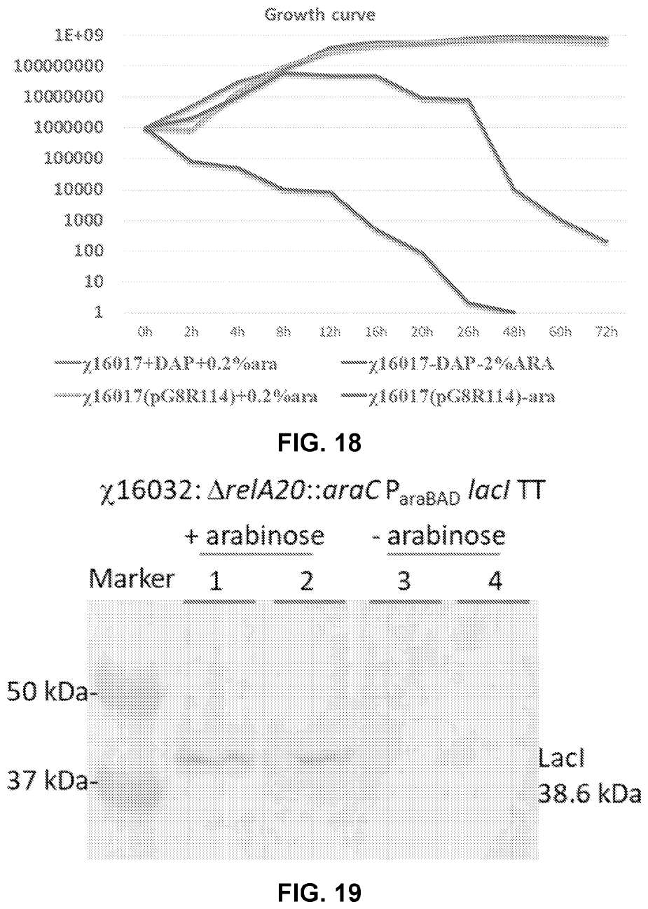

Strains with the asdA mutation have an obligate requirement for diaminopimelic acid (DAP), which is an essential constituent of the peptidoglycan layer of the bacterial cell wall. In environments lacking DAP, i.g., animal tissues, asdA mutants undergo lysis. The deletion of asdA gene has been used to develop antibiotic-sensitive RAEV vaccine strains for fish. The regulated delayed lysis system first developed for the RASV vector systems, relies on regulated expression of the asdA and murA genes encoding enzymes required for synthesis of DAP and muramic acid, respectively, essential components of the peptidoglycan layer of the bacterial cell wall. Since the product of the murA gene is phosphorylated and so cannot be taken up by bacteria, murA deletions are lethal. It is therefore necessary to create a conditional-lethal murA mutation by replacing the chromosomal murA promoter with the araC P_{araBAD} activator-promoter. The ΔP_{murA180}::TT araC P_{araBAD} murA mutation was introduced into wild-type E. piscicida J118 to yield χ16016, which only grew in broth containing arabinose. When χ16016 was deprived of arabinose, cells lysed. χ16016 had the same infectivity in Epithelioma papulosum cyprini (EPC) fish cells, as did J118. The ΔP_{murA180}::TT araC P_{araBAD} murA mutation was introduced into the AasdA strain χ16000 to yield χ16017. As expected, growth of χ16017 was dependent on both DAP and arabinose (FIG. 18). Regulated programmed cell lysis is achieved by using χ16017 and complementing the two mutations (asdA and murA) by a plasmid vector pG8R114 that possesses asdA and murA genes under control of araC P_{araBAD} as discussed in detail below. In the absence of arabinose, the P_{araBAD} promoters cease to be active, with no further synthesis of AsdA and MurA. These concerted activities lead to cell lysis (FIG. 18). This regulated delayed lysis process is central to conferring biological containment with no prolonged persistence of bacterial cells in vivo for more than several weeks and no survival when released into aqueous environments.

RAEV vaccine strains with ΔP_{crp68}::TT araC P_{araBAD} crp and ΔP_{fur170}::TT araC P_{araBAD} fur deletion-insertion mutations both confer a regulated delayed attenuation phenotype. Growth of strains with these mutations in the presence of arabinose leads to transcription of the respective virulence genes, but gene expression ceases in the absence of arabinose such that the Crp and Fur gene products are diluted out in vivo as a consequence of vaccine cell growth and division. Since Crp is needed to regulate expression of many genes needed for metabolic activities, its absence is attenuating but also proficient in inducing immunity. The Fur protein regulates expression of over 80 genes involved in iron acquisition, but when it becomes absent there is an accelerated uptake of iron, which in excess is toxic and thus attenuating. In the literature it has been shown that E. piscicida strains with ΔP_{crp68}::TT araC P_{araBAD} crp or ΔP_{fur170}:TT araC P_{araBAD} fur deletion-insertion mutations are attenuated by both bath and i.e. routes of inoculation into zebrafish and induce protective immunity to challenge with the parent J118 strain.

### Regulated Delayed Antigen Synthesis

Herein is disclosed a regulated delayed antigen synthesis system (RDAS) to minimize the negative effects of antigen expression on the host strain and to enhance immunogenicity. A tightly regulated araC P_{araBAD} lacI TT cassette was constructed and integrated into the E. piscicida chromosome in the re/A gene. ReIA was chosen as the integration site because ΔrelA mutations are not attenuating for virulence, nor do they affect colonization. ΔrelA also uncouples dependence of growth on protein synthesis so enhances regulated delayed lysis efficiency. The native lacI gene has a GTG start codon and an AGGG Shine-Dalgarno sequence, leading to synthesis of only 5 to 10 molecules each generation. This system includes a lacI gene expressed from the arabinose regulated araC P_{araBAD} promoter with a GTG start codon and an AGGG Shine-Dalgarno sequence. The chromosomal repressor LacI serves to regulate expression from a plasmid promoter, P_{trc}, that directs synthesis of mRNA that is translated to result in antigen synthesis. In the presence of arabinose, LacI is produced, and binds to P_{trC}, blocking antigen synthesis. In vivo, an arabinose-poor environment, the concentration of LacI decreases with each cell division, allowing increased antigen synthesis. The ΔrelA20::araC P_{araBAD} lacI TT insertion mutation was introduced into wild-type E. piscicida J118 to yield χ16032, which synthesizes LacI only in broth containing arabinose. However, in absence of arabinose no detectable LacI synthesis was observed (FIG. 19).

Determination of Lethal Dose 50 (LD₅₀) by i.e. Injection

To determine the LD₅₀ of E. piscicida strains J118, χ16016, χ16032, and χ16035, ten-fold serial dilutions of E. piscicida fresh cultures were made in sterile BSG, and the concentration of bacteria was determined by the spread-plate method. Fish were i.e. injected in a dose of 10 μL of BSG containing different concentrations of CFU/fish (Table 9) with 10 fish in each group (two replicate tanks). Fish were monitored daily and the health status was documented for over 3 weeks (χ16032 and χ16035) or 6 weeks (J118, χ16016) and the Reed and Muench method was used to calculate the LD₅₀ values. Survived fish from group J118 and χ16016 were monitored for longer period of time to determine if there are any delayed effects due to vaccine strains. After 6 weeks, kidney and intestine were collected (five fish from each group), homogenized in 200 μL of BSG and plated on LB agar plates containing 12 μg/mL of colistin with or without arabinose. The plates were incubated at 30° C. for 48 h. There were no E. piscicida colonies obtained. This indicates that all the E. piscicida strains were cleared by the host cells due to their display of biological containment attributes.

TABLE 9
LD₅₀ Study of E. piscicida wild type and
vaccine strains in catfish by i.c. injection

| | | | | | |
|---|---|---|---|---|---|
| | | | | Mortality | |
| Dose CFU/fish | mortality | deaths | survivals | % | LD₅₀ |

J118 (Wild-Type E. piscicida)

| | | | | | |
|---|---|---|---|---|---|
| 9 × 10² | 1/10 | 1 | 9 | 5 | 1.7 × 10⁴ |
| 9 × 10³ | 4/10 | 4 | 6 | 38 | |
| 9 × 10⁴ | 8/10 | 8 | 2 | 87 | |
| 9 × 10⁵ | 10/10 | 10 | 0 | 100 | |
| 9 × 10⁶ | 10/10 | 10 | 0 | 100 | |

χ16016 (ΔP_{murA180}::TT araC P_{araBAD} murA)

| | | | | | |
|---|---|---|---|---|---|
| 4 × 10⁴ | 0/10 | 0 | 10 | 0 | 5 × 10⁶ |
| 4 × 10⁵ | 0/10 | 0 | 10 | 0 | |
| 4 × 10⁶ | 4/10 | 4 | 6 | 40 | |
| 4 × 10⁷ | 10/10 | 10 | 0 | 100 | |
| 4 × 10⁸ | 10/10 | 10 | 0 | 100 | |

χ16032 (ΔrelA20::araC P_{araBAD} lacI TT)

| | | | | | |
|---|---|---|---|---|---|
| 1 × 10⁴ | 2/10 | 2 | 8 | 20 | 4.5 × 10⁴ |
| 1 × 10⁵ | 6/10 | 6 | 4 | 66 | |
| 1 × 10⁶ | 10/10 | 10 | 0 | 100 | |

χ16033 (ΔasdA10, ΔrelA20::araC P_{araBAD} lacI TT)

| | | | | | |
|---|---|---|---|---|---|
| 1.2 × 10⁵ | 4/10 | 4 | 6 | 33 | 2.5 × 10⁵ |
| 1.2 × 10⁶ | 8/10 | 8 | 2 | 85 | |
| 1.2 × 10⁷ | 10/10 | 10 | 0 | 100 | |

χ16035 (ΔasdA10; ΔP_{fur170}::TT araC P_{araBAD}
fur; DP_{crp68}::TT araC P_{araBAD} crp ΔrelA20::araC P_{araBAD} lacI TT)

| | | | | | |
|---|---|---|---|---|---|
| 1.4 × 10⁵ | 0/10 | 0 | 10 | 0 | 7 × 10⁶ |
| 1.4 × 10⁶ | 2/10 | 2 | 8 | 14 | |
| 1.4 × 10⁷ | 6/10 | 6 | 4 | 66 | |

Plasmid Vectors for A. hydropohila Antigen Synthesis

All plasmids confer the regulated delayed lysis in vivo phenotype and employ the balanced-lethal vector-host concept for stable plasmid maintenance to ensure that live RAEV strains are sensitive to all antibiotics and thus unable to disseminate antibiotic resistance when RAEVs are used in non-enclosed environments. The regulated lysis vectors depicted in FIGS. 10A-10C. All have P_{frc}-regulated synthesis of protective antigens for delivery by cell lysis and araC P_{araBAD}-regulated murA and asd genes with GTG start codons to decrease translation efficiency. Transcription terminators (TT) flank all plasmid domains for controlled lysis, replication and gene expression so that expression in one domain does not affect activities of another domain. Levels of induced cellular immunities are often highest with lower levels of delivered antigen whereas induction of antibody responses are better with delivery of higher amounts of antigen. To achieve the optimal levels of antigen synthesized, plasmid vectors with different copy numbers were used. In this regard, in addition to the regulated delayed lysis vectors depicted in FIGS. 10B-10C with the pBR on (high copy number) and p15A on (moderate copy number) FIG. 10A, vectors with low copy number (pSC101 on) both without and with the much-improved optimized β-lactamase signal sequence were available. Recombinant antigen delivery is achieved during lysis of the RAEVs or by action of host phagocytic cells breaking down RAEV cells in the case of using vectors such as pG8R111 (FIG. 10B). Use of pG8R114 (FIG. 10C) vector with the fusion of antigens to the bla SS (T2SS) leads to the delivery of antigens to the periplasm, resulting in an increased production of outer membrane vesicles (OMVs) that enhance immunogenicity and antibody production against delivered antigens. In addition, use of Type 2 secretion for protective antigen delivery also leads to protective antigen released into the supernatant fluid surrounding cells to also enhance the level of induced immune responses due to the enhanced production of outer membrane vesicles containing synthesized protective antigens. The time of onset of in vivo lysis can be controlled, in part, by using plasmids with different copy numbers. These plasmids, especially those with Type 2 and 3 secretion systems (T2SS; T3SS), can also be used to synthesize and deliver different effector molecules to enhance induction of innate immune responses.

### Materials and Methods

Bacterial culture conditions. Bacteriological media and components are from Difco (Franklin Lakes, NJ). Antibiotics and reagents are from Sigma (St. Louis, MO). LB broth and Brain Heart Infusion (BHI) are used as complex media for bacterial propagation. MacConkey agar, and Shotts & Waltman agar supplemented with D-lactose (0.5% w/v), D-maltose (1% w/v) or L-galactose (0.05% w/v), are used to count bacteria from fish tissues. When required, the media are supplemented with 1.5% agar, colistin sulfate (Col; 12.5 μg/mL), ampicillin (Amp; 50 μg/mL), chloramphenicol (Cm; 25 μg/mL), kanamycin (Km; 50 μg/mL), or tetracycline (Tet; 12.5 μg/mL). Bacterial growth is monitored spectrophotometrically and/or by plating. Strain culture and storage at −80° C. were according to standard methods.

Molecular and genetic procedures. Methods for DNA isolation, restriction enzyme digestion, DNA cloning and use of PCR for construction and verification of vectors are standard. DNA sequence analyses were performed at the UF Interdisciplinary Center for Biotechnology Research (ICBR). All oligonucleotide and/or gene segment syntheses were done commercially with codon optimization to enhance translational efficiency in Edwardsiella and stabilize mRNA to “destroy” RNase E cleavage sites to prolong mRNA half-life. Insertion of defined deletion or deletion insertion mutations were accomplished by conjugational transfer of suicide vectors to E. piscicida J118 using standard methods with the suicide vector donor strains χ7213 and χ7378. Plasmid constructs were evaluated by DNA sequencing, for genetic stability during 50 generations of growth under permissive conditions and for ability to specify synthesis of A. hydrophila proteins using gel electrophoresis and western blot analyses.

RAEV genetic modification. The RAEV strains are modified by insertion of defined unmarked deletion and deletion-insertion mutations as described above using selection for drug resistance and then counter selection for allelic exchange using LB agar plates supplemented with 10% sucrose. RAEV strains were fully characterized before immunization studies. Lipopolysaccharide (LPS) profiles are evaluated by SDS-PAGE and visualized by silver staining, to ensure that rough variants were not inadvertently selected. Plasmid profiles were verified by alkaline lysis and agarose gel (0.5%) electrophoresis. Comparative growth analyses were conducted since the objective was to have single and multiple mutant strains grow at the same rate and to the same density as the wild-type parental strains when grown under permissive conditions. The strains were evaluated by using API 20E tests (bioMerieux) for metabolic attributes and for sensitivity to antibiotics and drugs. Genotypes were confirmed by PCR. Motility tests are used to reveal presence or absence of functional flagella.

Cell biology. The ability of various constructed RAEV-Ah strains to attach to, invade into and survive in channel catfish ovary (CCO) cell lines and head kidney primary macrophages (HKDM) were quantified by established methods.

Power analysis, animal numbers, and statistics. In tests of RAEV-TiLV constructs, statistically significant results were expected. Previously, groups of 50 zebrafish were i.e. vaccinated with RAEV. The fish were then challenged with virulent E. piscicida strain, which gave 60% survival. Similar or better survival in these experiments was observed. Based on these data, power analysis was performed; the result shows that groups of 25 fish were sufficient for statistically significant results for 60% survival (Table 10). Thus, 25 fish were needed per group. Significant differences in percentage mortality between treatment groups (vaccinated and control) and between replicates (tanks) were determined at P<0.05. Relative percent survival was calculated. Key experiments were repeated.

TABLE 10
Survival Rates for Control and Vaccinated Groups

| Proportion of Fish | Proportion of Fish | Sample Size |
|---|---|---|
| Surviving in Group 1 | Surviving in Group 2 | Required for |
| (Control) | (Vaccinated) | Each Group |
| 0.1 | 0.4 | 42 |
| 0.1 | 0.5 | 29 |
| 0.1 | 0.6 | 23 |
| 0.1 | 0.7 | 19 |
| 0.1 | 0.8 | 16 |

Assumptions: Power = 80%, α = 0.01, Proportion lost to follow-up = 0

Channel catfish infections and immunizations. All the described studies with catfish were performed in compliance with University of Florida approved IACUC protocol #202009533.

Channel catfish. Channel catfish fingerlings, approximately 1-2 months of age and weighing 1.5 to 2 g, were ordered through UF ACS and maintained at 26° C. in a catfish cultivation system. Fish were acclimated for 2 weeks under laboratory conditions. The light system was turned on at 7:00 am and off at 9:00 pm (14/10 h light/dark cycle). Fish were fed two times a day with commercial Purina Aquamax 300 fingerling food. Reverse osmosis (RO) water were conditioned by adding instant sea salt and sodium bicarbonate to maintain the conductivity between 300-400 μS and pH between 7.0 and 7.4. The water was changed daily.

Estimation oflethal dose 50 (LD₅₀) in catfish. The LD₅₀ dose of each RAEV-Ah strain was determined by following the same procedure as described above. In brief, to determine the LD₅₀ of RAEV-Ah strains, ten-fold serial dilutions of RAEV fresh cultures were made in sterile BSG, and the bacterial concentration were determined by the spread plate method. Based on this preliminary data, RAEV vaccine strain χ16035 has ˜700 times increased LD₅₀ compared to the wild-type E. piscicida J118 by i.e. injection. χ16016 is safe at 4×10⁸ CFU/mL by bath immersion. This information helped to determine the minimum and maximum dose for i.e. injection and bath immersion. Fish (for each group: n=25/tank in three replicate) were infected by the bath or intracoelomic (i.e.) route with different dose of bacterial suspension. Mortality were recorded daily for 6 weeks, and LD₅₀ values were calculated by the method described by Reed and Muench (Reed and Muench, 1938).

Vaccination trials. For vaccination trials, fish were divided into experimental and naïve control groups. Experimental groups were treated with E. piscicida RAEV strains delivering A. hydrophila antigens to determine if they confer immunity to A. hydrophila infection. The naïve control group were treated with a RAEV carrying a plasmid (pG8R114 or pG8R111) that does not encode A. hydrophila antigen. An additional control group were treated with only BSG (vaccine diluent). To get the statistically significant results, each group had 3 replicate tanks and every tank had 25 fish (n=25, three replicate). The final experiment were repeated three times.

Intracoelomic vaccination. Fish were anesthetized prior to handling using 200 mg/L of buffered MS-222. i.e. vaccination is done using a 22G needle and tuberculin syringe. Catfish were immunized by i.e. vaccination with RAEV-Ah and RAEV-vector control strains. Four to six weeks post-immunization, fish were challenged by i.e. with a lethal dose of highly virulent ML-10-51K. Mortalities were recorded daily. Fish that survive for 6 weeks of post-challenge, and show no signs of external infection, are considered to have survived. Moribund fish were euthanatized by treatment with 500 mg/L of buffered MS-222. Fish were necropsied to evaluate presence of E. piscicida in kidney, spleen, and liver and formalin fixed tissues prepared for histologic evaluation.

Bath (immersion) vaccination. Fish were infected by whole body immersion for 2 h at 26° C. in 250 mL of water per fish containing RAEV-Ah doses in CFU based on results of i.e. vaccination trials. Control fish are treated similarly, but are exposed to the RAEV-vector control. Four to six weeks post-immunization, fish were challenged with a lethal dose of highly virulent WT A. hydrophila ML-10-51K by immersion or i.e. challenge. Mortalities were recorded daily. Fish that survive for 6 weeks post-challenge, and show no signs of external infection, are considered to have survived. Randomly selected fish were necropsied for the experiments.

Evaluation of cross-immune protection of RAEV-Ah vaccine strain. All Gram-negative bacteria possess surface-associated outer membrane proteins (OMPs), some of which have long been recognized as potential vaccine candidates. Antigenic cross-reactivity of OMPs has been reported among Gram-negative bacteria. In this study, conserved OMPs were used as vaccine candidates (Table 7). The amino acid sequences of these antigens are >90% homologue (except AGM42919) among different serotype of the target pathogens. Thus, Ah antigens delivered by RAEV strains (RAEV-Ah) are likely to confer significant protection among Aeromonas and Edwardsiella species. To evaluate the hypothesis, the final RAEV-Ah vaccine strain was immunized to catfish by i.e. or bath immersion (as stated above). After four weeks, the humoral immunogenicity and cross-reaction in serum was evaluated by ELISA. Vaccinated fish were challenged at 4 to 6 weeks of post vaccination by i.e. or bath with the most virulent WT A. hydrophila strain ML-10-51K. The RAEV-Ah strain which gives significant protection against most virulent WT strain, they should protect against less virulent strains (TN-97-08 and ALG 15-097). Next, challenges were carried out with E. piscicida J118 and E. ictaluri J100, individually. This result demonstrated the homologous and heterologous cross immune protection ability of RAEV-Ah strains.

Cross-reaction properties of the anti-RAEV-Ah sera. These studies were performed among A. hydrophila strains (TN-97-08, ML-10-51K and ALG 15-097, E. piscicida strains (J118, C07-087 and E. ictaluri strains J100 (Reference) by the whole cell ELISA assay. Catfish sera collected at four weeks of post vaccination were serially diluted, and the ratio of test group and negative control group greater than 2:1 was considered as positive. This result was confirmed the ability of RAEV-Ah strains in cross immune protection.

Monitoring immune responses. Immune responses in best performing E. piscicida RAEV-Ah vaccinated fish were evaluated in catfish fingerlings. RAEV-Ah with the regulated delayed lysis attribute could be used as an adjuvant to recruit innate immunity by delivering highly conserved microbial structures that include lipopolysaccharide, flagella, CpG DNA, lipoprotein, peptidoglycan (PGN) fragments Idiaminopimelic acid (DAP) and muramyl dipeptide (MDP)). The detection of conserved microbial motifs relies on the two major classes of PRRs including Toll-like receptors (TLRs) and NOD-like receptors (NLRs). The activation of TLRs and NLRs by RAEV-Ah was investigated by using HEK-Blue cells expressing mouse TLR4, TLR5, TLR8, TLR9, NOD1 and NOD2 (all from InvivoGen) with NFkB-inducible secreted embryonic alkaline phosphatase (SEAP) reporter gene. The ability of RAEV in inducing the innate immunity has previously been reported. The ability of RAEV-Ah to stimulate an immune response was evaluated, the expression of the genes encoding IL-1R, IL-6, IL-8, and TNF-α in catfish tissues (gill, kidney, intestine, spleen and blood) were analyzed by qRT-PCR following a published protocol. The activation of adaptive immune-related genes, including those for CD4 and CD8 T-cell coreceptors (cd4-1, cd4-2, cd8-α, cd8-β and mhc-II) was studied by qRT-PCR. Serum and mucosal IgM titer were studied by ELISA. The protective efficacy of RAEV-Ah mediated by adaptive immune responses (T and B cell-mediated) was evaluated in vaccinated fish against virulent A. hydrophila.

Pathologic examination of fish tissues. Methods utilized for microscopic examination of fish tissues followed a standard necropsy protocol. Freshly dead animals were thoroughly examined, with all viscera and major organ systems properly sampled and placed in ten percent neutral buffered formalin or Davidson's fixative for no less than 12 h. Tissue scrapes, smears, and bacterial cultures may be obtained at this time, as needed. Fixed tissues were sliced, processed routinely, and embedded in paraffin. 3 to 5 μm thick sections were stained with hematoxylin and eosin for microscopic examination. Each tissue was examined by a veterinary anatomic pathologist. Both experimental and control groups, and both i.e. and immersion groups, were necropsied at the end of the experiment to assess if gross pathological changes in surviving fish might exist as a result of delayed or chronic effects of vaccinations. The gross pathological changes in both experimental and control groups were assessed, and both i.e. and immersion groups, vaccinated fish tissues at day 120 of post vaccinations to know if there are any delayed or chronic effects of vaccinations.

Tissue persistence in catfish. Tissue persistence of RAEV-Ah strains after vaccination was determined. Briefly, four replicate tanks were stocked with 10 fingerlings each and fish were infected by i.e. injection or bath immersion as outlined above. One fish from each tank was randomly sampled at 0 h, 3 d, 5 d, 10 d, 15 d, 20 d, and 25 d post infection, and euthanatized. Liver, spleen, and anterior kidney tissues were collected aseptically and homogenized in 1 mL PBS. The homogenate was diluted serially and plated on LB agar plates with or without arabinose in triplicate. The plates were incubated at 37° C. for 24 h, the viable bacterial colonies were enumerated, and the number of CFU per gram homogenate calculated for each organ. The colony numbers per gram of tissue were analyzed using the statistical procedure.

Understanding innate and adaptive immune responses in catfish. Phagocytosis is an important early step in triggering the adaptive immune responses. To characterize the effects of safe and efficacious vaccines on the function of APCs, active phagocytosis and bacterial killing were assessed in catfish macrophages as described previously. Expression levels of genes relevant for innate and adaptive immunity was also assessed by quantitative real-time PCR following established procedures. Further, effects of vaccines on catfish B cells were explored by following protocols in recent publications.

Quantitative real-time PCR (qRT-PCR) analysis to determine differential expression of immune genes. Immune related genes play an important role in immune protection. It is therefore important to investigate the responses of these genes following vaccination. Activation of proinflammatory cytokines leads to regulating immunoglobulin synthesis in teleosts. The effect of vaccination in inducing genes encoding tumor necrosis factor-α (tnf-α), interleukin 1β (il-1β), interleukin-6 (il-6) and interleukin 8 (il-8) was investigated in different immune organs i.e., gills, kidney, intestine, spleen and blood by qRT-PCR following established procedures.

ELISA. IgM is the primary systemic antibody in teleost fish and is crucial for channel catfish in contributing to mucosal immune responses, as catfish lack the IgT antibody isotype. The vaccine-specific IgM titer is an important immune parameter of immunized fish. The vaccine-specific IgM antibody levels of vaccinated fish were compared to the control fish at 4- and 6-weeks post immunization. Control and immunized fish were sampled individually at 4- or 6-weeks post immunization. Serum and mucus from gill and skin were collected from immunized and control fish as described before. E. piscicida outer membrane proteins (OMPs) were purified as described previously and A. hydrophila antigens were purified by the His-tag purification procedure. An enzyme-linked immunosorbent assay (ELISA) was carried out to assay antibodies in serum and mucus against E. piscicida LPS or OMPs and A. hydrophila antigens. Purified antigen samples were diluted in bicarbonate coating buffer and the 96-well flat-bottom microtiter plates were coated overnight at 4° C. with 100 μL/well of LPS or OMPs. Plates were washed three times with 200 μL of PBS-0.05% Tween (PBS-T) per well, and blocked with 5% bovine serum albumin (BSA) for 1 h at room temperature. After washing, plates were incubated with a series of diluted catfish anti-serum/mucus at room temperature for 2 h. The plates were washed three times with PBS-T and incubated with mouse anti-catfish IgM monoclonal antibody (Aquatic Diagnostics Ltd) for 1 h at room temperature. Plates were then incubated with biotinylated goat anti-mouse IgG (Southern Biotechnology Associates, Birmingham, AL) for 1 h at room temperature. After incubation of wells with a streptavidin-alkaline phosphatase conjugate (Southern Biotechnology) for 1 h at 37° C., p-nitrophenyl phosphate (PNPP, Thermo Fisher Scientific) was added for color development. The optical density (OD) units were read at 405 nm using an automated ELISA plate reader (model EL311SX; Biotek, Winooski, VT).

### Objectives

Screen candidate A. hydrophila antigens delivered by RAEV strain to induce protective immunity in catfish against challenge with virulent wild-type (WT) A. hydrophila. 7 RAEV strains synthesizing different A. hydrophila antigens have already been designed and constructed. The immunogenicity and attenuation of all constructs encoding A. hydrophila antigens in the disclosed RAEV vaccine strains were first evaluated in comparison to a control with the empty pG8R114 vector by both bath and i.e. inoculation of catfish administered at various doses. Based on preliminary data, RAEV vaccine strain χ16035 has ˜700 times increased LD₅₀ compared to the wild-type E. piscicida J118 by i.e. injection. χ16016 is safe at 4×10⁸ CFU/mL by bath immersion. These results helped to determine the minimum and maximum dose of other RAEV by i.e and bath vaccination. Based on above results, the immunization doses were determined. Vaccine strains were evaluated in catfish for protection against wild-type E. piscicida and A. hydrophila challenge. The efficacy of the RAEV strains were tested first by i.e. vaccination and then by bath immersion. i.e. vaccination ensures that each fish receives a defined dose of a RAEV strain and allowed the minimum efficacious dose to be determined. Bath immersion represents the optimum method of RAEV delivery; however, the protective RAEV dose may differ from that determined by i.e. vaccination.

I.C. vaccination. The i.e. vaccination dose was determined based on the LD₅₀ dose of the RAEV-Ah strain. Groups of 150 catfish fingerlings were i.e. vaccinated with RAEV-Ah vaccine and 150 catfish with the RAEV vector control (pG8R114). Vaccinated catfish fingerlings were kept in triplicate tanks (n=50/tank, 3 replicates). Before initiation of the experiment, 10 fish in each group were tagged by injection of colored “Visible Implant Elastomer” (Northwest Marine Technologies, Shaw Island, WA), which allowed these fish to be individually identified over the course of the experiment. Sera were collected before vaccination and at 6 weeks post vaccination from VIE tagged fish to determine antibody titers against E. piscicida and A. hydrophila antigen. If serum samples taken at 4 to 6 weeks after i.e. vaccination show only low titers of Abs that recognize A. hydrophila antigen or E. piscicida, then a booster i.e. vaccination using the same dose was administered, which elicited a strong antibody (Ab) response. Ab titers were determined again four weeks later. At 7 weeks post vaccination, or 11 weeks if a booster vaccination is required, the RAEV-Ah and RAEV vector control groups were split (each tank) into two groups of 25 fish (in each group; n=25/tank, 3 replicate). One group was challenged with E. piscicida wild-type J118 with ˜5×10⁵ CFU/fish which is 50× of LD₅₀ dose while another group (n=25/tank, 3 replicate) was challenged with virulent A. hydrophila initially with ˜3.5×10⁴ CFU/fish which is 50× of LD₅₀ dose. Survival and mortality were monitored for 6 weeks of post challenge. Fish vaccinated with RAEV-Ah should be protected against A. hydrophila and E. piscicida. Fish vaccinated with the RAEV vector control should be protected against E. piscicida, but not A. hydrophila and serve as the A. hydrophila naïve control.

Bath and oral vaccination. RAEV-Ah bath immersion vaccination doses were determined based on the result of LD₅₀ study. Groups of 150 catfish fingerlings were bath vaccinated with RAEV-Ah vaccine and 150 catfish with the RAEV vector control (pG8R114). Vaccinated catfish fingerlings were kept in triplicate tanks (n=50/tank in triplicate). Before initiation of the experiment, 10 fish in each group were tagged by VIE injection to allow these fish to be individually tracked over the course of the experiment. Sera were collected before and at 6 weeks post vaccination to determine antibody titers against E. piscicida and A. hydrophila. At 6 weeks post vaccination the fish were split into two groups of 25 fish (in each group; n=25/tank in triplicate). One group was challenged with E. piscicida J118 wild type (50×LD₅₀). The second group was challenged with A. hydrophila wild type (50×LD₅₀). Survival and mortality were monitored for 6 weeks post challenge. Fish vaccinated with the same RAEV strain carrying a plasmid lacking the sequence encoding the A. hydrophila served as a naïve control and were expected to succumb to A. hydrophila challenge infection.

Evaluate further improved RAEV strains with mutations shown to enhance ability of Salmonella-vectored vaccines to induce protective immunity. The final goal is to develop a safe and effective live attenuated bacterial vaccine. LPS is the major surface membrane component present in almost all Gram-negative bacteria. In a previous study in Salmonella, modification of lipid A can reduce the inflammatory responses and enhance the safety without compromising the vaccine efficiency. It was previously demonstrated that catfish respond to E. ictaluri LPS and LPS modification reduce tissue damage without changing the colonization ability in catfish model. It was proposed to add ΔgalE and ΔpagL::TT araC P_{araBAD} waaL mutations to the RAEV system to achieve the reversible synthesis of lipopolysaccharide (LPS) O-antigen. In absence of arabinose waaL gene expression ceases and eliminated the enzyme that joins the LPS O-antigen chain to the LPS core resulting in a moderate-rough phenotype and also renders vaccine strain totally attenuated. The ΔgalE mutant made a truncated LPS core and no O-antigen when grown in the absence of galactose but made a complete LPS when grown in medium with 0.1% galactose. It was therefore predicted that the ΔgalE mutation would provide one means of achieving a regulated delayed attenuation in vivo phenotype (as it does in Salmonella) since there is no free unphosphorylated galactose in fish flesh. This attenuation hypothesis was tested. ΔgalE and ΔwaaL ΔpagL::araC P_{araBAD} waaL strains were characterized by quantifying the endotoxins by Limulus Amebocyte Lysate assay. The best mutation confirming regulated delayed attenuation without compromising the immunogenicity was added to both lysis (χ16034) and non-lysis (χ16035) vaccine vector strains. A. hydrophila protective antigens were delivered by these two final strains.

Comparative evaluation of regulated lysis vectors to deliver protective antigens by Type 2 and 3 secretion systems to achieve further enhancement of induced protective immunity. 9 A. hydrophila antigens were selected (Tables 7 and 8). These are immunogenic and give significant protection in fish (references are in table). The regulated lysis vectors were used with and without signal sequence (SS) pG8R114 and pG8R111 (FIGS. 10A-10C) to specify synthesis of A. hydrophila antigens. These vectors have P_{trc}-regulated synthesis of protective antigens for delivery by cell lysis. Use of pG8R114 vector with the fusion of antigens to the bla SS leads to the delivery of antigens to the periplasm, resulting in an increased production of outer membrane vesicles (OMVs) that enhance immunogenicity and antibody production against delivered antigens. The time of onset of in vivo lysis can be controlled, in part, by using plasmids with different copy numbers. These plasmids, especially those with Type 2 and 3 secretion systems (T2SS; T3SS), can be used to synthesize and deliver different effectors molecules to enhance induction of innate immune responses. All A. hyrophila antigens were codon-optimized based on E. piscicida highly expressed genes codon usages. 11 plasmids encoding A. hydrophila antigen genes were successfully constructed in pG8R114, which has the Type 2 SS. (Table 8). Antigen synthesis in E. coli and E. piscicida was confirmed by western blotting. pG8R114 and pG8R111 plasmids were also constructed encoding all other antigens. All the constructs were introduced into the final vaccine delivery strain. All phenotypic properties were fully characterized, including ultimate lysis when RAEVs are grown in the absence of arabinose. Plasmid constructs were evaluated by DNA sequencing, for genetic stability during 50 generations of growth under permissive conditions and for ability to specify synthesis and secretion of A. hydrophila antigen using gel electrophoresis and western blot analyses. His-tagged proteins were produced and used to obtain anti-protein rabbit and/or fish antibodies for western blot analyses. Choose the best performing construct for each antigen specified in the construct.

Construction of E. piscicida-vectored vaccines to deliver multiple A. hydrophila protective antigens and demonstration that RAEV vectored vaccine against A. hydrophila also protects against wild-type E. piscicida. All the constructs were first evaluated in comparison to a control with the empty pG8R114 or pG8R111 vector to determine full attenuation by both bath and i.e. inoculation of catfish administered at various doses. The antigen-specific IgM titers were also determined. The best vaccine strains for protection against A. hydrophila and infection in catfish were selected. Plasmids were constructed with operon fusions to enable delivery of best two to three A. hydrophila antigens from the same vector (pG8R114 or pG8R111). These RAEV constructs were evaluated alone and in combination depending on the number and efficacy of the selected antigens in conferring protective immunity. The objective was to validate one or two RAEV constructs that give maximal protection against both A. hydrophila and E. piscicida.

### Example 4: Construction of E. piscicida ΔendA Strain

The modification of E. piscicida vaccine vector strains to be used as vectors to deliver DNA vaccines requires several modifications to enhance their efficacy for successful DNA vaccine delivery.

Endonuclease-I is responsible for an extensive deoxyribonucleic acid (DNA) breakdown. Thus, it is of great advantage to use RAEV strains in which endonuclease-I activity is reduced to increase the DNA vaccine plasmid survival upon its release into the host cell.

To construct a ΔendA in-frame deletion mutant strain, the recombinant pG8R8044 suicide vector carrying the linked flanking regions was constructed as described previously. The defined deletion mutation comprises deletion of DNA including the ATG start codon and TAG stop codon. PCR fragments of 474-bp upstream and 405-bp downstream of the endA gene flanking region were amplified using E. piscicida genomic DNA as template, with primers: endA1FW(KpnI)/endA2RV and endA3FW/endA4RV(XmaI), respectively. The two PCR products were fused by overlapping PCR with primers endA1FW(KpnI)/endA4RV(XmaI), and the products were cloned into the KpnI and XmaI sites of the suicide vector pRE112. To construct E. piscicida endA mutants, the suicide plasmid pG8R8044 was conjugationally transferred from E. coli χ7213 to E. piscicida strains. Strains containing single-crossover plasmid insertions were isolated on LB agar plates containing Cm. Loss of the suicide vector after the second recombination between homologous regions (i.e., allelic exchange) was selected by using the sacB-based sucrose sensitivity counter-selection system. The colonies were screened for Cms and by PCR using primers endA1FW(KpnI)/endA4RV(XmaI). The resultant E. piscicida strain containing the ΔendA11 mutation was named as χ16029. Primers used are shown in Table 11:

TABLE 11
Primers Used to Construct ΔendA Strain

| | | SEQ |
|---|---|---|
| | | ID |
| Primer | Sequence | NO. |
| endA1FW | catggtaccATCCGTTCGGCATCCAGACGCAC | 56 |
| (KpnI) | | |
| endA2RV | GTTTTAGAATCCCCGATCCCGCCGCTGCCACTC | 57 |
| endA3FW | GGGATCGGGGATTCTAAAACGGTGTTGGAATGAg | 58 |
| endA4RV | catcccgggATCCCCCATGAGCTGGCGCATCTG | 59 |
| (XmaI) | | |

Characterization of E. piscicida endA Mutant

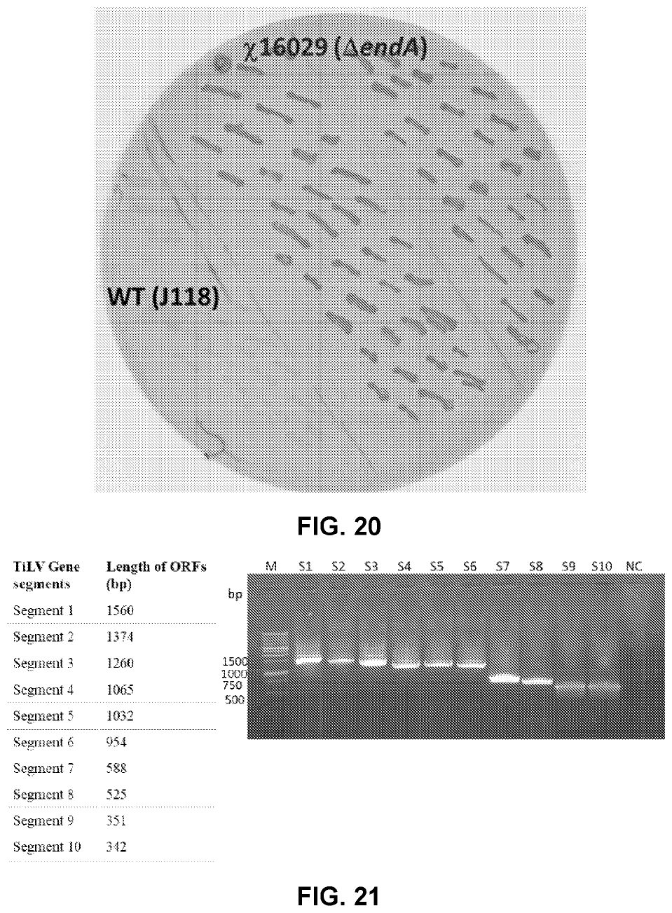

Detection of DNA degradation by methyl green staining: E. piscicida wild-type and endA mutant strains were stained with methyl green following the protocol described by Michel Wright. Bacterial colonies were patched on a LB agar plate in a glass petri dish and were grown overnight at 37° C. The plate was covered with 10 mL of toluene and then incubated for 14 h at 37° C. After incubation, excess toluene was poured out and the plate was air dried for 15 min. Next, colonies were transferred onto Whatman 3 Qualitative 9.0 cm filter paper discs. The paper was then placed in a container with an acidic methyl green solution (2.5 mL 2% methyl green, 650 mL 95% ethanol, 347.5 mL ddH₂O) and placed on a shaker for 1 hr. The paper was removed from the solution and air-dried. Wild-type colonies pick up little to no stain, while ΔendA mutants stain clearly (FIG. 20).

### Example 5: Construction of E. piscicida ΔtrxL and ΔfliC mutant strains

The decreased E. piscicida-induced pyroptosis/apoptosis phenotype allows the DNA vaccine time to traffic to the nucleus for efficient synthesis of encoded protective antigens.

Deletion of the flagellin gene fliC of E. tarda results in decreased cytotoxicity for infected macrophages and does not attenuate its virulence in a fish model of infection. Trxlp is an important virulence effector in E. piscicida. A reduced death cell death/pyroptosis, caspase-1 activation and IL-1β secretion during Δtrxlp infection in macrophage, compared with the wild-type E. piscicida.

Construction of ΔtrxlP: To construct a ΔtrxlP in-frame deletion mutant strain, the recombinant pG8R8047 suicide vector carrying the linked flanking regions was constructed as described previously. The defined deletion mutation comprises deletion of DNA starting from 21 bp downstream of the ATG start codon to the TAA stop codon. PCR fragments of 983-bp upstream and 1040-bp downstream of the trxlP gene flanking region were amplified using E. piscicida genomic DNA as template, with primer pairs: trxlP-1 F-XbaI/trxlP-2R-kpnl and trxlP-3F-KpnI/trxlP-4R-Sacl, respectively. The two PCR products were fused by overlapping PCR with primers trxlP-1F-XbaI/trxlP-4R-Sacl and the products were cloned into the XbaI and Sacl sites of the suicide vector pRE112. To construct E. piscicida trxlP mutants, the suicide plasmid pG8R8047 was conjugationally transferred from E. coli χ7213 to E. piscicida strains. Strains containing single-crossover plasmid insertions were isolated on LB agar plates containing Cm. Loss of the suicide vector after the second recombination between homologous regions (i.e., allelic exchange) was selected by using the sacB-based sucrose sensitivity counter-selection system. The colonies were screened for Cm^{s} and by PCR using primers trxlP-1F-XbaI/trxlP-4R-Sacl. The resultant E. piscicida strain containing the ΔtrxlP mutation was named as χ160³⁷. Primers used for this mutation are listed below.

Construction of ΔfliC: To construct a ΔfliC in-frame deletion mutant strain, the recombinant pG8R8048 suicide vector carrying the linked flanking regions was constructed as described previously. The defined deletion mutation comprises deletion of DNA including the ATG start codon and TAG stop codon. PCR fragments of 978-bp upstream and 1002-bp downstream of the fliC gene flanking region were amplified using E. piscicida genomic DNA as template, with primer pairs: fliC-1-FW(XbaI)/fliC-2R and fliC-3F/fliC-4-RV(XmaI), respectively. The two PCR products were fused by overlapping PCR with primers fliC-1-FW(XbaI)/fliC-4-RV(XmaI) and the products were cloned into the XbaI and XmaI sites of the suicide vector pRE112. To construct E. piscicida ΔfliC mutants, the suicide plasmid pG8R8048 was conjugationally transferred from E. coli χ7213 to E. piscicida strains. Strains containing single-crossover plasmid insertions were isolated on LB agar plates containing Cm. Loss of the suicide vector after the second recombination between homologous regions (i.e., allelic exchange) was selected by using the sacB-based sucrose sensitivity counter-selection system. The colonies were screened for Cm^{s} and by PCR using primers fliC-1-FW(XbaI)/fliC-4-RV(XmaI). The resultant E. piscicida strain containing the ΔfliC mutation was named as χ16038. Primers used to develop ΔfliC mutation are listed below.

TABLE 12
Primers Used to Construct ΔtrxIP and ΔfliC Strains

| | | SEQ ID |
|---|---|---|
| Description | Sequence | NO. |
| trxIP-1F-XbaI | catTCTAGAgcaaagaccgtcggtggcttcttgtgata | 60 |
| trxIP-2R-kpnI | catGGTACCtaaagtgctcagggttttcatgatgctttc | 61 |
| trxIP-3F-KpnI | catGGTACCggacgccccatggagataagcctgctgacac | 62 |
| trxIP-4R-SacI | catGAGCTCctgcaggaaaaactggcgcgatagcatgccg | 63 |
| fliC-1-FW(XbaI) | catTCTAGAtagcatacttagttgtaccagcatcat | 64 |
| fliC-2R | aaagcacacttcgcgcattaccgcgtgcgtggccc | 65 |
| fliC-3F | taatgcgcgaagtgtgctttccttcgaatgttgc | 66 |
| fliC-4-RV(XmaI) | catCCCGGGgcgggtgatatcgatgccgttgagtgt | 67 |

### Cytotoxicity Assays by Lactate Dehydrogenase (LDH) Release

EPC cells were grown up to 70-80% confluence in a 24-well cell culture plate. Cell were washed and incubated in antibiotic free medium. EPC cells were infected with E. piscicida strains (J118, Δtrxlp and ΔfliC) with 10:1 (bacteria/EPC cell) for one hour. EPC Cells were washed with prewarmed phosphate-buffered saline (PBS) and incubated in complete EMEM medium with 100 μg/ml gentamicin for 1 h. The EPC cells were then incubated in complete EMEM medium supplemented with 10 μg/ml gentamicin for the rest of the experiment. The CytoTox 96 Cytotoxicity assay (Promega) was used to determine cell death by measuring the release of Lactate dehydrogenase (LDH). The supernatants of infected cells were collected at 3, 6, 12 and 20 hr and centrifuged for 5 min at 500×g in a swinging-bucket centrifuge to remove cell debris. LDH release from uninfected/untreated cells was used for background subtraction. Reduced cytotoxicity was observed in bath Δtrxlp and ΔfliC treated cells compare to the wild-type J118 treated cells at 3 h and 6 h. This result confirmed the decreased Edwardsiella piscicida-induced pyroptosis/apoptosis phenotype of RAEV strains with Δtrxlp and ΔfliC mutations.

### Example 6: Tilapia Lake Virus Vaccine

Development of E. piscicida DNA Vaccine Vector Strains

A series of E. piscicida mutant strains derived from J118 have been constructed, an R plasmid-cured derivative of the highly virulent E. piscicida E1B202, which has been sequenced. J118 is sensitive to all antibiotics. Suicide vector technologies were used with pRE112 to insert all the deletion and deletion-insertion mutations in the DNA vaccine delivery vector strain χ16041, which has the following genotype: ΔasdA10 ΔP_{murA180}:TT araC P_{araBAD} murA ΔP_{crp68}::TT araC P_{araBAD} crp ΔP_{fur170}::TT araC P_{araBAD} fur ΔendA11 ΔtrxL20 ΔfliC20. This strain displays regulated delayed lysis, regulated delayed attenuation, increased DNA vector survival or stability and reduced pyroptosis/apoptosis (Table 13). These are the required feature for an ideal DNA vaccine delivery system. Strains with the asdA mutation have an obligate requirement for diaminopimelic acid (DAP), which is an essential constituent of the peptidoglycan layer of the bacterial cell wall. In environments deprived of DAP, i.e., animal tissues, asdA mutants undergo lysis (Table 13). Deletion of the asdA gene has previously been exploited to develop antibiotic-sensitive strains of live attenuated recombinant bacterial vaccines for fish. Introduction of an Asd⁺ plasmid (which is a property of the DNA vaccine vector to be used) into a ΔasdA mutant makes the bacterial strain plasmid-dependent. This dependence on the Asd⁺ plasmid vector creates a balanced-lethal complementation between the bacterial strain and the recombinant plasmid. The regulated delayed lysis system first developed for the RASV vector systems, relies on regulated expression of the asdA and murA genes encoding enzymes required for synthesis of DAP and muramic acid, respectively, essential components of the peptidoglycan layer of the bacterial cell wall. Since the product of the murA gene is phosphorylated that cannot be taken up by bacteria, murA deletions are lethal. It is therefore necessary to create a conditional-lethal murA mutation by replacing the chromosomal murA promoter with the araC P_{araBA}D activator-promoter. The ΔendA11 mutation eliminates the endonuclease that potentially cleaves and thus destroys DNA vaccine vectors during their release from lysing E. piscicida vaccine strains. E. piscicida strains induce host cell death during infection by several mechanisms and this is likely to diminish transcription of a DNA vaccine after trafficking to the nucleus. Trxlp and FliC promotes the inflammasome activation during E. piscicida infection in macrophages which activates caspase-1 leading to cell death by a process termed pyroptosis/apoptosis. The ΔtrxL and ΔfliC mutations eliminate two means by which E. piscicida, after invading into host cells, induces pyroptosis, which would very much decrease the ability of DNA vaccines to have their antigen-encoding genes expressed, thus rendering them ineffectual. Additional strains are found in Table 14.

TABLE 13
Strain construction and LD₅₀ of E. piscicida vaccine strains

| | | | LD₅₀ in zebrafish |
|---|---|---|---|
| Strain # | Genotype | Features | by I.C. injection |
| J118 | Wild-Type E. piscicida | Wild-type | 1.1 × 10⁴ |
| χ16000 | ΔasdA10 | lysis | 7.5 × 10⁶ |
| χ16010 | ΔP_{crp68}::TT araC P_{araBAD} crp | (i) Regulated delayed attenuation | 1.8 × 10⁵ |
| χ16012 | ΔP_{fur170}::TT araC P_{araBAD} fur | (i) Regulated delayed attenuation | 2 × 10⁴ |
| χ16016 | ΔP_{murA180}::TT araC P_{araBAD} murA | (i) Regulated delayed lysis | 8 × 10⁴ |
| χ16035 | ΔasdA10; ΔP_{fur170}::araC P_{araBAD} | (i) Regulated delayed attenuation | 1.4 × 10⁷ |
| | fur TT; ΔP_{crp68}::araC P_{araBAD} crp | (ii) Regulated delayed antigen | |
| | TT; ΔrelA20::araC P_{araBAD} lacI | synthesis | |
| | TT | | |
| χ16029 | ΔendA11 | (i) Increase plasmid survival | 1 × 10⁴ |
| χ16037 | ΔtrxlP20 | (i) Reduced pyroptosis/apoptosis | 8 × 10³ |
| χ16038 | ΔfliC20 | (i) Reduced pyroptosis/apoptosis | 5 × 10⁴ |
| χ16039 | Δtrxlp20 ΔfliC20 | (i) Reduced pyroptosis/apoptosis | 7 × 10⁴ |
| χ16041 | ΔasdA10 ΔP_{murA180}::TT araC | (i) Regulated delayed lysis | 7 × 10⁷ |
| | P_{araBAD} murA P_{crp68}::TT araC | (ii) Regulated delayed attenuation | |
| | P_{araBAD} crp ΔP_{fur170}::TT araC | (iii) Increased plasmid survival or | |
| | P_{araBAD} fur ΔendA11 ΔtrxL20 | stability | |
| | ΔfliC20 | (iii) Reduced pyroptosis/apoptosis | |

TABLE 14
List of E. piscicida mutant strains

| SI. | Strain | | Parent |
|---|---|---|---|
| # | Name | Relevant characteristics/Genotype | strain |
| 1 | χ16000 | ΔasdA10 | J118 |
| 2 | χ16001 | Δfur11 | J118 |
| 3 | χ16002 | Δpmi21 | J118 |
| 4 | χ16003 | Δcrp22 | J118 |
| 5 | χ16004 | ΔznuA23 | J118 |
| 6 | χ16005 | ΔgalE24 | J118 |
| 7 | χ16006 | Δwaal25 | J118 |
| 8 | χ16007 | Δcrp22ΔznuA23 | χ16003 |
| 9 | χ16008 | ΔP_{rfaH13}::TT araC P_{araBAD} rfaH | J118 |
| 10 | χ16009 | ΔP_{rfaH23}::TT araC P_{araBAD} rfaH | J118 |
| 11 | χ16010 | ΔP_{crp68}::TT araC P_{araBAD} crp | J118 |
| 12 | χ16011 | ΔP_{crp78}::TT araC P_{araBAD} crp | J118 |
| 13 | χ16012 | ΔP_{fur170}::TT araC P_{araBAD} fur | J118 |
| 14 | χ16013 | ΔpstS26 | J118 |
| 15 | χ16014 | ΔP_{pstS27}::TT araC P_{araBAD} pstS | J118 |
| 16 | χ16015 | ΔasdA10; ΔP_{fur170}::TT ₐᵣₐaraC P_{BAD} fur | χ16000 |
| 17 | χ16016 | ΔP_{murA180}::TT araC P_{araBAD} murA | J118 |
| 18 | χ16017 | ΔasdA10; ΔP_{murA180}::TT araC P_{araBAD} murA | χ16000 |
| 19 | χ16018 | ΔasdA10; ΔP_{fur170}::TT araC P_{araBAD} fur, ΔP_{murA180}::TT araC P_{araBAD} | χ16015 |
| | | murA | |
| 20 | χ16019 | ΔAroA11 | J118 |
| 21 | χ16020 | ΔphoP12 | J118 |
| 22 | χ16021 | ΔasdA10; ΔP_{fur170}::TT araC P_{araBAD} fur, ΔAroA11 | χ16015 |
| 23 | χ16022 | ΔasdA10; ΔP_{fur170}::TT araC P_{araBAD} fur; ΔP_{crp68}::TT araC P_{araBAD} | χ16015 |
| | | crp | |
| 24 | χ16023 | ΔP_{rfaD}::TT araC P_{araBAD} rfaD | J118 |
| 25 | χ16024 | ΔP_{lacI28}::TT araC P_{araBAD} lacI | J118 |
| 26 | χ16025 | ΔasdA10; ΔP_{fur170}::TT araC P_{araBAD} fur, ΔP_{murA180}::TT araC P_{araBAD} | χ16018 |
| | | murA; ΔP_{lacI28}::TT araC P_{araBAD} lacI | |
| 27 | χ16026 | ΔasdA10; ΔP_{fur170}::TT araC P_{araBAD} fur; ΔP_{crp68}::TT araC P_{araBAD} | χ16022 |
| | | crp; ΔP_{lacI28}::TT araC P_{araBAD} lacI | |
| 28 | χ16027 | ΔasdA10; ΔP_{fur170}::TT araC P_{araBAD} fur, ΔP_{murA180}::TT araC P_{araBAD} | χ16018 |
| | | murA; ΔP_{crp68}::TT araC P_{araBAD} crp | |
| 29 | χ16028 | ΔAroA11ΔphoP12 | χ16019 |
| 30 | χ16029 | ΔendA11 | J118 |
| 31 | χ16030 | ΔasdA10; ΔP_{fur170}::TT araC P_{araBAD} fur, ΔP_{murA180}::TT araC P_{araBAD} | χ16027 |
| | | murA; ΔP_{crp68}::TT araC P_{araBAD} crp, ΔendA11 | |
| 32 | χ16031 | asdA10; ΔP_{fur170}::TT araC P_{araBAD} fur, ΔP_{murA180}::TT araC P_{araBAD} | χ16018 |
| | | murA; ΔendA11 | |
| 33 | χ16032 | ΔrelA20::araC P_{araBAD} lacI TT | J118 |
| 34 | χ16033 | ΔasdA10, ΔrelA20::araC P_{araBAD} lacI TT | χ16000 |
| 35 | χ16034 | ΔasdA10; ΔP_{fur170}::TT araC P_{araBAD} fur, ΔP_{murA180}::TT araC P_{araBAD} | χ16027 |
| | | murA; ΔP_{crp68}::TT araC P_{araBAD} crp ΔrelA20::araC P_{araBAD} lacI TT | |
| 36 | χ16035 | ΔasdA10; ΔP_{fur170}::TT araC P_{araBAD} fur; ΔP_{crp68}::TT araC P_{araBAD} crp | χ16022 |
| | | ΔrelA20::araC P_{araBAD} lacI TT | |
| 37 | χ16037 | ΔtrxlP20 | J118 |
| 38 | χ16038 | ΔfliC20 | J118 |
| 39 | χ16039 | ΔfliC20 ΔtrxlP20 | χ16037 |
| 40 | χ16040 | ΔasdA10; ΔP_{fur170}::TT araC P_{araBAD} fur, ΔP_{murA180}::TT araC P_{araBAD} | χ16030 |
| | | murA; ΔP_{crp68}::TT araC P_{araBAD} crp, ΔendA11 Δtrxlp20 | |
| 41 | χ16041 | ΔasdA10; ΔP_{fur170}::TT araC P_{araBAD} fur, ΔP_{murA180}::TT araC P_{araBAD} | χ16030 |
| | | murA; ΔP_{crp68}::TT araC P_{araBAD} crp, ΔendA11 ΔfliC20 | |
| 42 | χ16042 | ΔrelA21::araC P_{araBAD} lacI TT (Weak SD) | J118 |
| 43 | χ16043 | ΔasdA10 ΔrelA21::araC P_{araBAD} lacI TT (Weak SD) | χ16000 |

TiLV Virus Cultivation, RNA Isolation, and cDNA Pjreparation and Cloning of TiLV-ORFs

The TiLV isolate (WVVL18053-01A) was used for this study and has been described previously (AI-Hussinee et al. 2018). TiLV isolate from a frozen stock inoculated onto a 175 cm² flask containing confluent striped snakehead (SSN-1; Eli clone) cells. The SSN-1 cells were maintained at 2° C. and grown in L-15 media (Leibovitz; Gibco, USA) containing 10% fetal bovine serum (FBS; Gibco, USA) with 1× antibiotic/antimycotic (AA; Gibco, USA), resulting in a concentration of 100 IP penicillin ml-1, 100 μg streptomycin ml-1, and 0.25 μg amphotericin B ml-1. After CPE was complete, the supernatant was clarified by centrifugation at 5000×g (20 min at 10° C.).

Total RNA was isolated from the virus infected cell supernatant was by using TRIzol reagent (Invitrogen, USA), and reverse transcribed into first-strand cDNA using RevertAid First Strand cDNA Synthesis kit (Thermo Scientific). According to the full-length sequence of TiLV segments (GenBank under accession no. MH319378 to MH319387) the specific primers were designed for PCR amplification. Using prepared cDNA as template, all the segments of TiLV were successfully amplified (FIG. 21). The PCR product was purified and ligated into CloneJET vector by using CloneJET PCR Cloning Kit (Thermo Scientific). Selected clones were confirmed by PCR and sequencing.

Construction of TiLV Plasmid Constructs and Confirmation of TiLV Antigen Synthesis in E. piscicida

Plasmids. The regulated lysis vectors depicted in FIGS. 10B-10C both have Ptrc-regulated synthesis of protective antigens for delivery by cell lysis and araC P_{araBAD}-regulated murA and asd genes with GTG start codons to decrease translation efficiency. Recombinant antigen delivery is achieved during lysis of the RAEVs or by action of host phagocytic cells breaking down RAEV cells in the case of using vectors such as pG8R111 (FIG. 10B). Use of pG8R114 (FIG. 10C) vector with the fusion of antigens to the bla SS (T2SS) leads to the delivery of antigens to the periplasm, resulting in an increased production of outer membrane vesicles (OMVs) that enhance immunogenicity and antibody production against delivered antigens. In addition, use of Type 2 secretion for protective antigen delivery also leads to protective antigen released into the supernatant fluid surrounding cells to also enhance the level of induced immune responses.

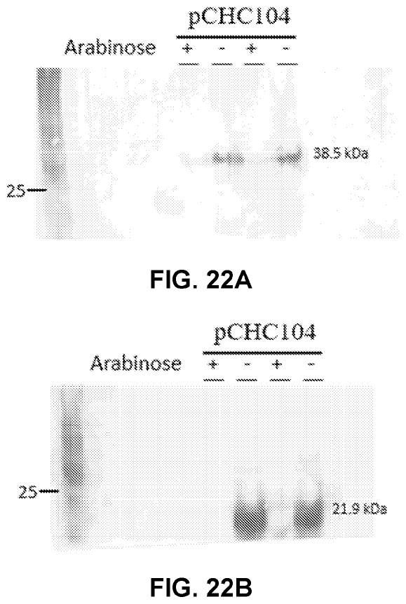

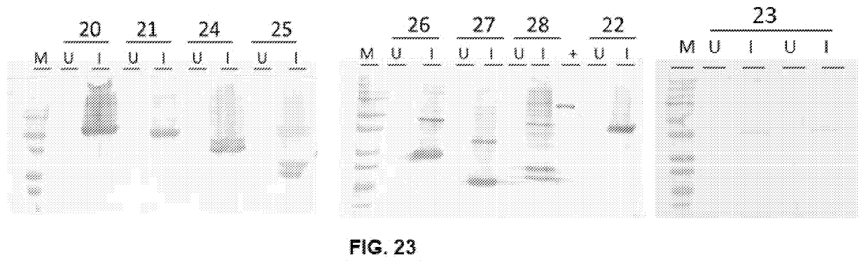

Western blotting. The TiLV gene segments were inserted into plasmid pG8R111 or pG8R114 (Table 15) and the resultant TiLV plasmids were electroporated individually into E. piscicida strain χ16035 (ΔasdA10 ΔP_{fur170}::TT araC P_{araBAD} fur ΔP_{crp}: TT araC P_{araBAD} crp ΔrelA::araC P_{araBAD} lacI TT). Synthesis of TiLV antigens were confirmed by western blotting (FIGS. 22A-22B). TiLV antigen synthesis was detected in χ16035(pCHC104) and χ16035(pCHC107) (FIGS. 22A-22B). The plasmid stability of these two strains growing under permissive conditions (presence of arabinose and DAP in media) was studied for up to 100 generations. These results showed that these plasmids were stable in E. piscicida vaccine strains and retained ability to produce the TiLV antigens. Additional plasmids used in these experiments are shown in Tables 16-18. Original DNA sequences of Segments 2-10 correspond to SEQ ID NOs.: 29-37; optimized DNA sequences of Segments 2-10 correspond to SEQ ID NOs. 38-46; and amino acid sequences of the optimized DNA sequences correspond to SEQ ID NOs. 47-55. Nucleotide sequences in the optimized sequences were selected based on E. piscicida codon usage. For Table 17, TiLV gene segments were inserted into the pET28(a)⁺ vector at Sail and Sad restriction enzyme site and the resultant plasmids were named as pG8R9020-pG8R9028. Antigen synthesis was confirmed by western blotting (FIG. 23).

TABLE 15
List of TiLV Plasmid Constructs

| | | Parent | | | Western |
|---|---|---|---|---|---|
| SL# | Plasmid# | Vector | Insert | Size | Blotting |
| 1 | pCHC101 | pG8R111 | Segment 6 | 34.1 kDa | Not detected |
| 2 | pCHC102 | pG8R111 | Segment 8 | 20.6 kDa | Very faint |
| 3 | pCHC104 | pG8R114 | Segment 6 | 38.5 kDa | Detected |
| 4 | pCHC105 | pG8R114 | Segment 8 | 24.9 kDa | Not detected |
| 5 | pCHC106 | pG8R111 | Segment SΔ3 | 17.5 kDa | Not detected |
| 6 | pCHC107 | pG8R114 | Segment SΔ3 | 21.9 kDa | Detected |

TABLE 16
Additional TiLV Plasmids

| Plasmid # | TiLV gene inserted | Parent plasmid |
|---|---|---|
| pG8R9020 | Segment 2 | pET28(a)⁺ |
| pG8R9021 | Segment 3 | pET28(a)⁺ |
| pG8R9022 | Segment 4 | pET28(a)⁺ |
| pG8R9023 | Segment 5 | pET28(a)⁺ |
| pG8R9024 | Segment 6 | pET28(a)⁺ |
| pG8R9025 | Segment 7 | pET28(a)⁺ |
| pG8R9026 | Segment 8 | pET28(a)⁺ |
| pG8R9027 | Segment 9 | pET28(a)⁺ |
| pG8R9028 | Segment 10 | pET28(a)⁺ |
| pG8R9029 | Segment 2 | pcDNA3.1/Myc-His-A |
| pG8R9030 | Segment 3 | pcDNA3.1/Myc-His-A |
| pG8R9031 | Segment 4 | pcDNA3.1/Myc-His-A |
| pG8R9032 | Segment 5 | pcDNA3.1/Myc-His-A |
| pG8R9033 | Segment 6 | pcDNA3.1/Myc-His-A |
| pG8R9034 | Segment 7 | pcDNA3.1/Myc-His-A |
| pG8R9035 | Segment 8 | pcDNA3.1/Myc-His-A |
| pG8R9036 | Segment 9 | pcDNA3.1/Myc-His-A |
| pG8R9037 | Segment 10 | pcDNA3.1/Myc-His-A |
| pG8R9040 | Segment 2 | pYA4545 |
| pG8R9041 | Segment 3 | pYA4545 |
| pG8R9042 | Segment 4 | pYA4545 |
| pG8R9043 | Segment 5 | pYA4545 |
| pG8R9044 | Segment 6 | pYA4545 |
| pG8R9045 | Segment 7 | pYA4545 |
| pG8R9046 | Segment 8 | pYA4545 |
| pG8R9047 | Segment 9 | pYA4545 |
| pG8R9048 | Segment 10 | pYA4545 |
| pCHC105 | Segment 8 | pG8R114 |
| pCHC107 | Segment 5 | pG8R114 |

TABLE 17
Insertion of Optimized TiLV Gene Segments into pET28(a)⁺ Vector

| | | | GenBank | Length | Length of | Expected |
|---|---|---|---|---|---|---|
| | Gene | Parent | Accession | of Gene | Protein | Protein Size |
| Plasmid # | Segments | Plasmid | No. | (bp) | (aa) | (kDa) |
| pG8R9020 | 2 | pET28(a)⁺ | MH319379 | 1374 | 457 | 56.8 |
| pG8R9021 | 3 | pET28(a)⁺ | MH319380 | 1260 | 419 | 53.0 |
| pG8R9022 | 4 | pET28(a)⁺ | MH319381 | 1065 | 354 | 43.7 |
| pG8R9023 | 5 | pET28(a)⁺ | MH319382 | 1032 | 343 | 43.3 |
| pG8R9024 | 6 | pET28(a)⁺ | MH319383 | 954 | 317 | 41.7 |
| pG8R9025 | 7 | pET28(a)⁺ | MH319384 | 588 | 195 | 27.1 |
| pG8R9026 | 8 | pET28(a)⁺ | MH319385 | 525 | 174 | 24.8 |
| pG8R9027 | 9 | pET28(a)⁺ | MH319386 | 351 | 116 | 18.5 |
| pG8R9028 | 10 | pET28(a)⁺ | MH319387 | 342 | 113 | 18.1 |

TABLE 18
TiLV DNA Vectors

| | | | | | Expected |
|---|---|---|---|---|---|
| | | | Length of | Length of | Protein |
| Plasmid | Gene | Parent | Gene | Protein | Size |
| # | Segments | Plasmid | (bp) | (aa) | (kDa) |
| pG8R9029 | 2 | pCDNA3.1 | 1374 | 457 | 56.8 |
| pG8R9030 | 3 | pCDNA3.1 | 1260 | 419 | 53.0 |
| pG8R9031 | 4 | pCDNA3.1 | 1065 | 354 | 43.7 |
| pG8R9032 | 5 | pCDNA3.1 | 1032 | 343 | 43.3 |
| pG8R9033 | 6 | pCDNA3.1 | 954 | 317 | 41.7 |
| pG8R9034 | 7 | pCDNA3.1 | 588 | 195 | 27.1 |
| pG8R9035 | 8 | pCDNA3.1 | 525 | 174 | 24.8 |
| pG8R9036 | 9 | pCDNA3.1 | 351 | 116 | 18.5 |
| pG8R9037 | 10 | pCDNA3.1 | 342 | 113 | 18.1 |

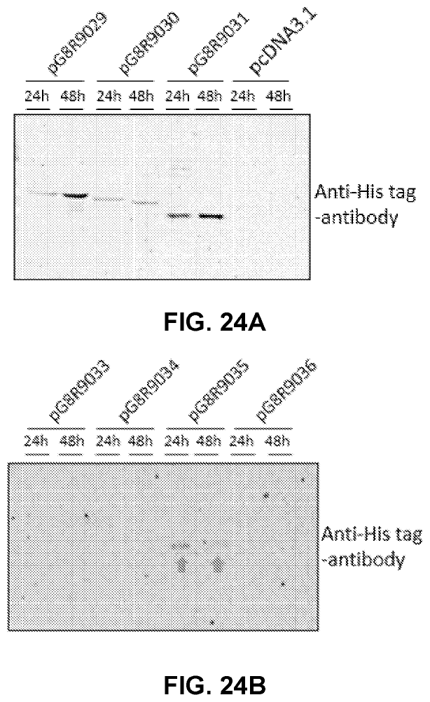

TiLV antigen synthesis by vector pcDNA3.1(+)/MYC-HIS/A in an HEK293T cell line. TiLV gene segments were inserted into pcDNA/3.1/A vector. HEK293T cell were transfected with pcDNA/3.1/A-TiLV plasmids using Lipofectamine 2000 and cells were incubated at 37° C. with 5% CO₂. After 24 and 48 hours of transfection, cells were harvested and lysed in sample loading buffer. Cell lysates were separated using 12% sodium dodecyl sulfate-polyacrylamide gel electrophoresis (SOS-PAGE) and transferred onto a nitrocellulose membrane for western blot Analysis. Anti-His-tag antibody was used for the detection of the TiLV antigen synthesis. SuperSignal™ West Pico PLUS Chemiluminescent Substrate was used to develop the blot. Cells transfected with pG8R9029, pG8R9030, and pG8R9031 (FIG. 24A) and pG8R9035 (FIG. 24B) showed positive signals.

Construction of DNA Vaccine Vector Encodinq EGFP and i-Antigen IAG52B an In Vitro Synthesis of Antigen

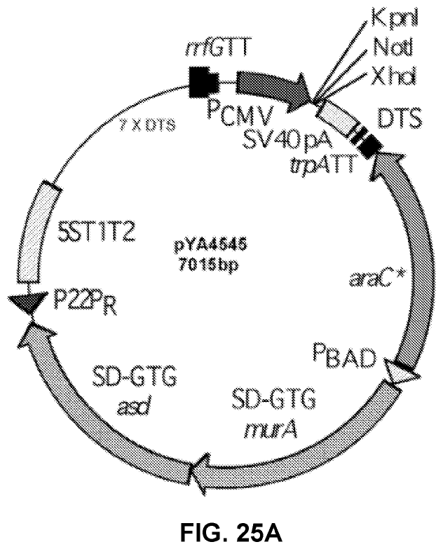

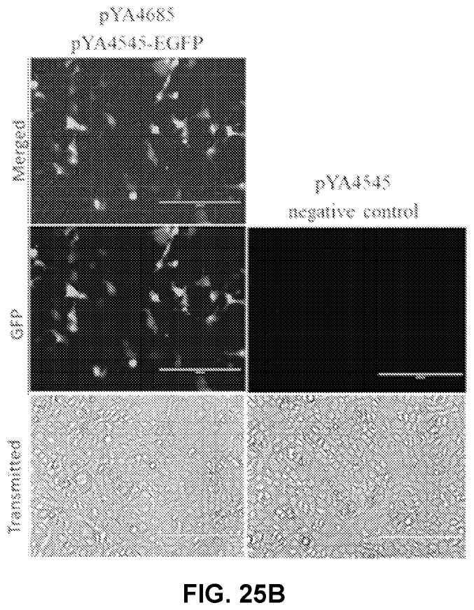

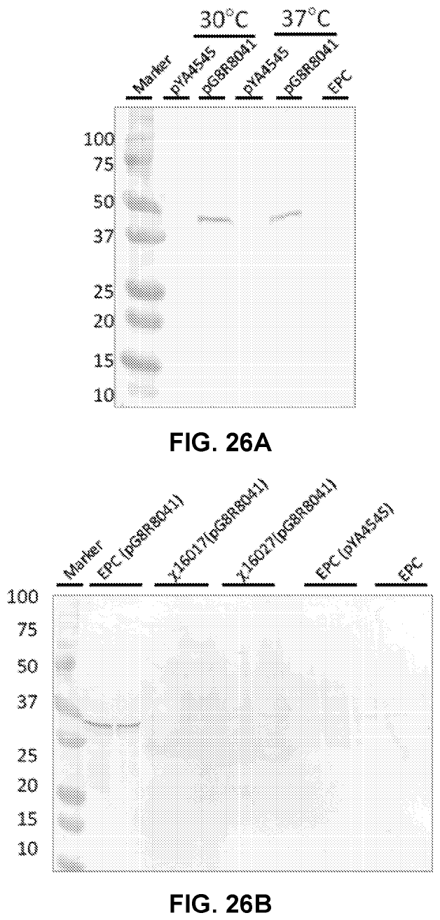

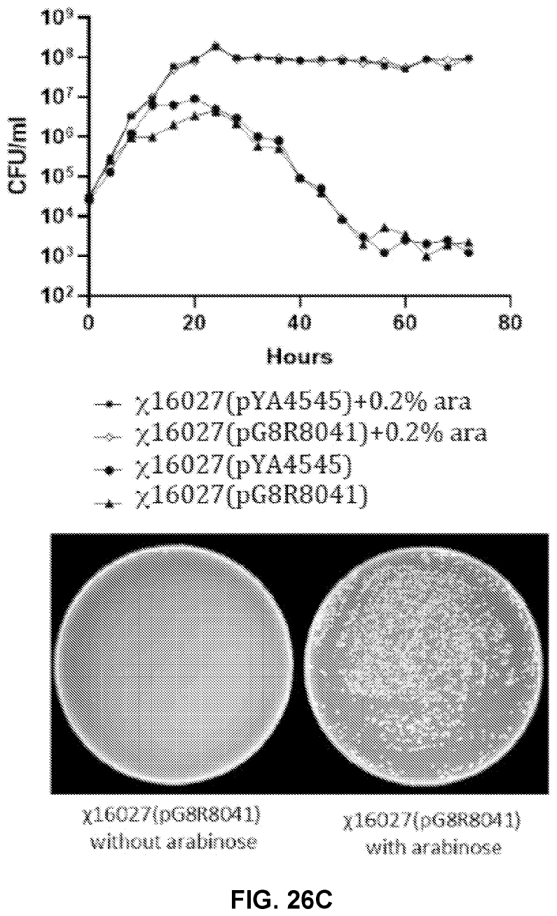

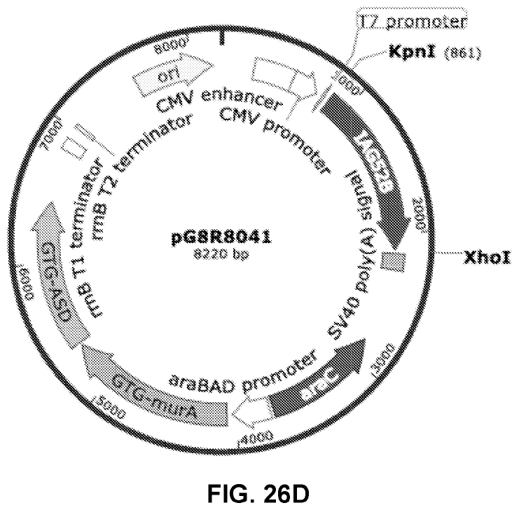

For this study, the improved DNA vaccine vector pYA4545 (FIG. 25A) has been used; this has the potential of plasmid nuclear import and resistance to attack by host nucleases. This plasmid complements the chromosomal asdA and murA imposed requirements of the RAEV regulated delayed lysis strains. To check the efficiency of DNA vaccine vector pYA4545 for antigen synthesis in a fish cell line, the plasmid pYA4545 (as control) and pYA4685 (EGFP gene was fused in frame with the Kozak sequence and inserted into vector pYA4545 downstream of the CMV promoter) were transfected into the Epithelioma papulosum cyprini (EPC) cells. Twenty hours after initiating the transfection, fluorescence of EGFP was visualized using fluorescent microscopy (objective 10×). Green fluorescence was observed in the pYA4685 transfected cells, whereas no fluorescent signal was observed in cells transfected with pYA4545 (FIG. 25B). This result confirms that the heterologous antigen can be efficiently synthesized by DNA vector pYA4545 in fish cells. Furthermore, the synthesis of surface antigen IAG52B) of fish parasite ciliated protozoan Ichthyophthirius multifiliis (Ich) was analyzed. The codon optimized sequence of IAG52B (which replaced the nonsense codon TAA specifying glutamine in the Ich parasite with the codon CAG specifying inclusion of the amino acid glutamine in proteins made in Edwardsiella) was PCR amplified and cloned into pYA4545 at KpnI and Xhol sites; the resultant plasmid was named as pG8R8041. pG8R8041 and pYA4545 were transfected into the EPC cell line and were incubated at 30° C. and 37° C. After 24 h of post transfection the synthesis of IAG52B was confirmed by western blotting using rabbit polyclonal antibody directed against the IAG52B i-antigen. Specific protein bands were observed in EPC cells transfected with pG8R8041 both in 30° C. and 37° C. incubated cells. There was no signal observed in control cells and in cells transfected with control plasmid pYA4545 (FIG. 26A). The results revealed that the IAG52B protein was successfully synthesized from the Ich-DNA vaccine plasmid pG8R8041 in fish cells. Then the Ich-DNA vaccine plasmid pG8R8041 and pYA4545 were electroporated into the E. piscicida regulated lysis strains χ16017 and χ16027 for vaccine delivery into fish. To confirm that the expression of the heterologous antigen gene by vector pYA4545 was tightly regulated, western blotting of lysates of E. piscicida lysis strains containing pG8R8041 was confirm that the expression of the heterologous antigen gene by vector pYA4545 were performed. There were no signals in the western blot indicating no antigen synthesis in the E. piscicida DNA vaccine vector strain (FIG. 26B). Then, lysis of the regulated lysis strain χ16027 carrying the plasmids pYA4545 and pG8R8041 was examined in vitro. Bacterial cells were cultured overnight in un-purple broth supplemented with 0.1% arabinose. Cell were harvested and diluted with un-purple broth with or without arabinose to the desired OD. Cultures were incubated at 30° C. with shaking and at every four hours intervals CFU/mL were determined by plating on LB-agar plates supplemented with 0.1% arabinose. The growth results indicated that the DNA vaccine strains can only grow in the presence of arabinose and that they lyse in the absence of arabinose (FIGS. 26C-26D). This feature of the disclosed lysis strains demonstrates complete biological containment.

### Objectives

Design of DNA vaccine vectors encoding TiLV gene segment. There are ten genomic sequences encoding ten proteins in the TiLV genome. The amino acid sequences of each of these proteins were thoroughly analyzed and have determined that eight would most likely be post-translationally modified by N-glycosylation at from 1 to 4 sites. Each protein has further been characterized as to its structural aspects (β sheets, β barrels, turns, coils, etc.) plus hydrophobicity, surface display and potential antigenicity (B and T cell epitope prediction) along each molecule. Each gene sequence has been modified to specify synthesis of a C-terminal His tag sequence to enable monitoring of synthesis of the fusion protein using a monoclonal antibody recognizing the His sequence. Additional N- and C-terminal nucleotide sequences were specified to enable use of specific restriction enzymes to correctly insert each protein encoding sequence into the pYA4545 DNA vaccine vector. Each construct was introduced into EPC or SSN-1 fish cells to verify synthesis of proteins of the predicted size. These studies were conducted with and without tunicamycin that blocks glycosylation so for each gene product the extent of glycosylation can be determined, as well as the change in protein mass as a consequence of this post translational modification. Each of the DNA vaccine constructs was then introduced into χ16041 (discussed above) to first evaluate for stability of constructs and then use for vaccination and TiLV challenge studies to identify the best vaccine candidate(s).

Construction of E. piscicida DNA vaccine vector strains to deliver TiLV-DNA vaccine and to be characterized for all genotypic and phenotypic traits including lysis, attenuation, and immunogenicity. All χ16041 constructs (carrying TiLV DNA vector) were first evaluated in comparison to a control with the empty pYA4545 vector to determine full attenuation by both bath and i.e. inoculation of tilapia administered at various doses. Which doses induce antibodies directed at the TiLV protein being expressed were also determined. These antibodies were used to determine which react with an extract of the TiLV virus since at present, which proteins are structural and included in virus particles is unknown. This also identified those proteins involved in virus replication and maturation but not included in the virus. Whether those antibodies induced to viral structural proteins are capable of neutralizing virus infection into EPC or SSN-1 cells was also determined.

Characterization and evaluation of immunogenicity of TiLV-DNA vaccines and ability to induce protective immunity to TiLV challenge in tilapia. The ability of the selected χ16041(pYA4545-TiLV gene constructs) to induce protective immunity in tilapia to TiLV challenge and generate TiLV neutralizing immunity was also investigated. All studies were repeated to ensure being able to reach statistically significant conclusions. Based on results during the studies of initial studies with tilapia, it was decided to construct vaccine strains delivering ability to induce immune responses to multiple TiLV proteins and the relevant studies were repeated.

### General Materials and Methods

RAEV characterization. RAEV strains were fully characterized before immunization studies. Lipopolysaccharide (LPS) profiles are evaluated by SDS-PAGE and visualized by silver staining, to ensure that rough variants were not inadvertently selected. Plasmid profiles were verified by alkaline lysis and agarose gel (0.5%) electrophoresis. Comparative growth analyses were conducted since the objective was to have single and multiple mutant strains grow at the same rate and to the same density as the wild-type parental strains when grown under permissive conditions. The strains were evaluated by using API 20E tests (bioMerieux) for metabolic attributes and for sensitivity to antibiotics and drugs. Genotypes were confirmed by PCR. Motility tests are used to reveal presence or absence of flagella.

Cell biology. The ability of RAEV-TiLV (RAEV carrying-TiLV DNA vector) strains to attach to, invade into and survive in fish cell line (EPC/CCO/SSN-1) was quantified by established methods. The fish cell line EPC (Epithelioma papulosum cyprinid from Pimephales promelas) purchased from the American Type Culture Collection (ATCC number CRL-2872) was used to quantify the TiLV antigen synthesis by transfecting DNA vaccine vectors encoding TiLV antigen genes.

Power analysis, animal numbers, and statistics. In tests of RAEV-TiLV constructs, statistically significant results were generated. Previously, groups of 50 zebrafish have been i.e. vaccinated with RAEV. The fish were then challenged with virulent E. piscicida strain, which gave 60% survival. Similar or better survival was seen in these experiments. Based on these data, power analysis was performed; the result shows that groups of 25 fish were sufficient for statistically significant results for 60% survival (Table 10). Thus, 25 fish were needed per group. Significant differences in percentage mortality between treatment groups (vaccinated and control) and between replicates (tanks) were determined at P<0.05. Relative percent survival was calculated. Key experiments were repeated.

Tilapia infections and immunizations. All the described studies with tilapia were performed in compliance with University of Florida approved Institutional Animal Care and Use Committee (IACUC Protocol #: 202011278 and 202009533).

Tilapia. Specific pathogen-free (SPF) Nile tilapia fingerlings, with no prior history of tilapia lake virus (TiLV) infection were purchased from Spring Genetics Inc., Miami, FL. Fish were acclimated for 30 d in tanks receiving single-pass dechlorinated municipal water maintained at 28±0° C. Water quality parameters (pH, ammonia, nitrite, hardness, dissolved oxygen) were recorded weekly using a Fish Farming Test Kit Model FF-1A (Hach) and a portable dissolved oxygen meter (Hach HQ30D). Fish were acclimated for 2 weeks under laboratory conditions. The light system was turned on at 7:00 am and off at 9:00 pm (14/10 h light/dark cycle). Fish were fed two times a day with commercial tilapia pellet food.

Estimation of lethal dose 50 (LD₅₀) in tilapia. To determine the LD₅₀ of RAEV strains (wild-type J118 and TiLV vaccine strains), ten-fold serial dilutions of RAEV fresh cultures were made in sterile PBS, and the bacterial concentration was determined by the spread plate method. Fish were infected by the bath or intracoelomic (i.e.) route with bacterial suspension per fish ranging from 1×10² to 1×10⁶ CFU per fish with 25 fish in each group. Mortality was recorded daily for 15 days, and LD₅₀ values were calculated by the method described by Reed and Muench (Reed and Muench, 1938).

Vaccination trials. For vaccination trials, fish were divided into experimental and naïve control groups. Experimental groups were treated with E. piscicida RAEV strains delivering TiLV DNA vector to determine if they confer immunity to TiLV infection. The naïve control group was treated with a RAEV carrying a plasmid (pYA4545) that does not encode TiLV DNA vector. An additional control group was treated with only PBS (vaccine diluent).

Intracoelomic vaccination. Fish were anesthetized prior to handling using 200 mg/I of buffered MS-222. i.e. vaccination is done using a 22G needle and tuberculin syringe. Tilapia were immunized by i.e. vaccination with RAEV-pYA4545-TiLV and RAEV-pYA4545 empty control strains. Four to six weeks post-immunization, fish were challenged by i.e. with a lethal dose (10×LD₅₀ dose) of highly virulent TiLV isolate VWL18053-01A. Mortalities were recorded daily. Fish that survive for 30 days post-challenge, and show no signs of external infection, are considered to have survived. Moribund fish were euthanatized by treatment with 500 mg/I of buffered MS-222. Fish were necropsied to evaluate presence of TiLV in kidney, spleen, and liver and formalin fixed tissues prepared for histologic evaluation.

Bath (immersion) vaccination. Tilapia were vaccinated by whole body immersion for 2 h at 28° C. in 500 mL of water per fish containing RAEV-pYA4545-TiLV doses in CFU based on results of i.e. vaccination trials. Control fish are treated similarly but are exposed to the RAEV-vector control. Four to six weeks post-immunization, fish were challenged with a lethal dose of highly virulent TiLV isolate VWL18053-01A by i.e. challenge. Mortalities were recorded daily. Fish that survive for 30 days post-challenge, and show no signs of external infection, are considered to have survived.

Evaluation of immune protection of RAEV-pYA4545-TiLV vaccine strain against E. piscicida. Antigens delivered by RAEV strains (RAEV-pYA4545-TiLV) are likely to confer significant protection against Edwardsiella species. To evaluate the hypothesis, the final RAEV-pYA4545-TiLV vaccine strain were immunized to tilapia by i.e. or bath immersion (as stated above). After four weeks, the humoral immunogenicity in serum was evaluated by ELISA. Vaccinated fish and control fish (only PBS group) were challenged at 4 to 6 weeks of post vaccination by i.e. or bath with the E. piscicida J118. This result demonstrated the immune protection ability of RAEV-pYA4545-TiLV strains to E. piscicida.

Monitoring immune responses. Immune responses in best performing RAEV-pYA4545-TiLV vaccine strain were evaluated in tilapia fingerlings.

Neutralizing assay. Collect sera from vaccinated and control fish and determine ability to interact with TiLV proteins and then neutralize TiLV ability to infect SSN-1 cells. The Serum Neutralization Test was performed as previously described. In brief, all serum samples were filtered and heat-inactivated for 30 min at 42° C. before testing. Diluted sera of 50 μL or L-15 supplemented with 0.15% BSA were added to 96-well plates (6 wells for one dilution). Next, 50 μL of TiLV (1×10⁵ TCID₅₀/mL) were added to each well of the plate and then incubated at 28° C. for 3 h. After incubation, 100 μL of SSN-1 cell suspension in L-15 supplemented with 0.15% BSA (2.0×10⁵ to 5.0×10⁵ cells) were added to each well of the plates that were then incubated at 28° C. for 7 days. The neutralizing antibody titer of the serum against TiLV was determined as the 50% endpoint of the serum that inhibited CPE in inoculated cells. Repeat studies to ensure statistically significant results.

Pathologic examination of fish tissues. Methods utilized for microscopic examination of fish tissues followed a standard necropsy protocol. Freshly dead animals were thoroughly examined, with all viscera and major organ systems properly sampled and placed in ten percent neutral buffered formalin or Davidson's fixative for no less than 12 h. Tissue scrapes, smears, and bacterial cultures may be obtained at this time, as needed. Fixed tissues were sliced, processed routinely, and embedded in paraffin. 3 to 5 μm thick sections were stained with hematoxylin and eosin for microscopic examination. Each tissue was examined by a veterinary anatomic pathologist.

Tissue persistence in tilapia. Tissue persistence of RAEV-pYA4545-TiLV strains after vaccination was determined. Briefly, five replicate tanks were stocked with 10 fingerlings each and fish were immunized by immersion as outlined above. one fish from each tank was randomly sampled at 0 h, 3 d, 5 d, 10 d, 15 d, 20 d, and 25 d post infection, and euthanatized. Liver, spleen, and anterior kidney tissues were collected aseptically and homogenized in 1 mL PBS. The homogenate was diluted serially and plated on BHI agar plates with arabinose in triplicate. The plates were incubated at 30° C. for 24 h, the viable bacterial colonies were enumerated, and the number of CFU per gram homogenate were calculated for each organ. The vaccine strain was confirmed by patching on arabinose plus and minus BHI agar plates. The colony numbers per gram of tissue were analyzed using the statistical procedure.

Understanding innate and adaptive immune responses in tilapia. Phagocytosis is an important early step in triggering the adaptive immune responses. To characterize the effects of safe and efficacious vaccines on the function of APCs, active phagocytosis and bacterial killing were assessed in tilapia macrophages as described previously. Expression levels of genes relevant for innate and adaptive immunity were also assessed by quantitative real-time PCR following established procedures. Further, effects of vaccines on tilapia B cells were explored by following protocols in recent publications.

Quantitative real-time PCR (qRT-PCR) analysis to determine differential expression of immune genes. Immune related genes play an important role in immune protection. It is therefore important to investigate the responses of these genes following vaccination. Activation of proinflammatory cytokines leads to regulating immunoglobulin synthesis in teleosts. The effects of vaccination in inducing genes encoding tumor necrosis factor-α (TNF-α), interleukin 1β (IL-1β), interleukin-6 (IL-6) and interleukin 8 (IL-8) were investigated in different immune organs i.e., gills, kidney, intestine, spleen and blood by qRT-PCR following established procedures.

ELISA. The soluble (secreted) form of IgM of teleost fish is generally found as a tetramer, unlike the mammalian pentameric secreted IgM. It constitutes the most abundant Ig class in fish serum and is the isotype that plays the most important role in the adaptive immune response at the systemic level. In addition, IgM also has a role in mucosal immune responses. The vaccine-specific IgM titer is an important immune parameter of immunized fish. The vaccine-specific IgM antibody levels of vaccinated fish were compared to the control fish at 4- and 6-weeks post immunization. Control and immunized fish were sampled individually at 4- or 6-weeks post immunization. Serum and mucus from gill and skin were collected from immunized and control fish as described before. An enzyme-linked immunosorbent assay (ELISA) was carried out to assay antibodies in serum and mucus against TiLV and E. piscicida. Inactivated TiLV was diluted (1:2) in bicarbonate coating buffer and the 96-well flat-bottom microtiter plates were coated overnight at 4° C. with 100 μL/well of inactivated TiLV. Plates were washed three times with 200 μL of PBS-0.05% Tween (PBS-T) per well, and blocked with 5% bovine serum albumin (BSA) for 1 h at room temperature. After washing, plates were incubated with a series of diluted tilapia anti-serum/mucus at room temperature for 2 h. The plates were washed three times with PBS-T and incubated with mouse anti-tilapia IgM monoclonal antibody (SKU: F04, Aquatic Diagnostics Ltd) for 1 h at room temperature. Plates were incubated with biotinylated goat anti-mouse IgG (Southern Biotechnology Associates, Birmingham, AL) for 1 h at room temperature. After incubation of wells with a streptavidin-alkaline phosphatase conjugate (Southern Biotechnology) for 1 h at 37° C., p-nitrophenyl phosphate (PNPP, Thermo Fisher Scientific) was added for color development. The optical density (OD) units were read at 405 nm using an automated ELISA plate reader (model EL311SX; Biotek, Winooski, VT).

Additional suicide plasmids useful in the disclosed system are described in Table 19:

TABLE 19
List of plasmids used for construction of E. piscicida mutant strains

| Plasmid # | Relevant characteristics |
|---|---|
| pRE112 | Suicide vector; sacB mobRP4 R6K ori; Cm^{r} |
| pYA3700 | Cloning vector containing TT araC P_{araBAD} cassette |
| pG8R8000 | Suicide vector to generate ΔasdA10, pRE112 Replican |
| pG8R8001 | Suicide vector to generate Δfur, pRE112 Replican |
| pG8R8002 | Suicide vector to generate Δpmi, pRE112 Replican |
| pG8R8003 | Suicide vector to generate Δcrp, pRE112 Replican |
| pG8R8004 | Suicide vector to generate ΔznuA, pRE112 Replican |
| pG8R8005 | Suicide vector to generate ΔgalE, pRE112 Replican |
| pG8R8006 | Suicide vector to generate Δwaal, pRE112 Replican |
| pG8R8007 | Suicide vector to generate ΔP_{rfaH13}::TT araC P_{araBAD} rfaH, pRE112 Replican |
| pG8R8008 | Suicide vector to generate ΔP_{rfaH23}::TT araC P_{araBAD} rfaH, pRE112 Replican |
| pG8R8009 | Suicide vector to generate ΔP_{crp68}::TT araC Pa_{raBAD} crp, pRE112 Replican |
| pG8R8010 | Suicide vector to generate ΔP_{crp78}::TT araC P_{araBAD} crp, pRE112 Replican |
| pG8R8024 | Suicide vector to generate ΔP_{fur170}::TT araC P_{araBAD} fur, pRE112 Replican |
| pG8R8025 | Suicide vector to generate ΔP_{murA180}::TT araC P_{araBAD} murA, PRE112 Replican |
| pG8R8026 | Suicide vector to generate ΔaroA11, pRE112 Replican |
| pG8R8027 | Suicide vector to generate ΔphoP12, pRE112 Replican |
| pG8R8031 | Suicide vector to generate ΔpstS26, pRE112 Replican |
| pG8R8032 | Suicide vector to generate ΔP_{pstS27}::TT araC P_{araBAD} pstS, PRE112 Replican |
| pG8R8033 | Suicide vector to generate ΔP_{rfaD}::TT araC P_{araBAD} rfaD, pRE112 Replican |
| pG8R8044 | Suicide vector to generate ΔendA11, pRE112 Replican |
| pG8R8046 | Suicide vector to generate ΔrelA20::araC P_{araBAD} lacI TT (GTG-lacI) |
| pG8R8047 | Suicide vector to generate Δtrxlp20, pRE112 Replican |
| pG8R8048 | Suicide vector to generate ΔfliC20, pRE112 Replican |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abdelhamed, H. et al, Evaluation of three recombinant outer membrane proteins, OmpA1, Tdr, and TbpA, as potential vaccine antigens against virulent Aeromonas hydrophila infection in channel catfish (Ictalurus punctatus), Fish & Shellfish Immunology 66 (2017) 480-486.
2. Abdel-Latif, H M R, et al, The nature and consequences of co-infections in tilapia: A review, Journal of Fish Diseases 43(6) (2020) 651-664.
3. Ahasan, M S, et al, Genomic characterization of tilapia lake virus isolates recovered from moribund Nile tilapia (Oreochromis niloticus) on a farm in the United States, Microbiology Resource Announcements 9(4) (2020) e01368-19.
4. Ahman, H, et al, Dose dependency of antibody response in infants and children to pneumococcal polysaccharides conjugated to tetanus toxoid, Vaccine 17(20-21) (1999) 2726-32.
5. Aich, N. et al, Tilapia Lake Virus (TiLV) disease: Current status of understanding, Aquaculture and Fisheries 7(1) (2022) 7-17.
6. AI-Hussinee, L. et al, Complete Genome Sequence of a Tilapia Lake Virus Isolate Obtained from Nile Tilapia (Oreochromis niloticus), Genome Announc 6(26) (2018) e00580-18.
7. Amal, M. et al, A case of natural co-infection of Tilapia Lake Virus and Aeromonas veronii in a Malaysian red hybrid tilapia (Oreochromis niloticus×O. mossambicus) farm experiencing high mortality, Aquaculture 485 (2018) 12-16.
8. Ameiss, K, et al, Delivery of woodchuck hepatitis virus-like particle presented influenza M2e by recombinant attenuated Salmonella displaying a delayed lysis phenotype, Vaccine 28(41) (2010) 6704-13.
9. Amemiya, K et al, Activation of Toll-Like Receptors by Live Gram-Negative Bacterial Pathogens Reveals Mitigation of TLR4 Responses and Activation of TLR5 by Flagella, Front Cell Infect Microbiol 11 (2021) 745325.
10. Amend, D, Potency testing of fish vaccines, Anderson D P, et al, editors. Fish biologics: serodiagnostics and vaccines. vol. 49., Basel, Switzerland:, 1981.
11. Armwood, A R, et al, Pathology and virulence of Edwardsiella tarda, Edwardsiella piscicida, and Edwardsiella anguillarum in channel (Ictalurus punctatus), blue (Ictalurus furcatus), and channel×blue hybrid catfish, Journal of Fish Diseases, 2022, 45:1683-1698.
12. Arpaia, N, et al, TLR Signaling Is Required for Salmonella typhimurium Virulence, Cell 144(5) (2011) 675-688.
13. Ashraf, S. et al, Protective cellular responses elicited by vaccination with influenza nucleoprotein delivered by a live recombinant attenuated Salmonella vaccine, Vaccine 29(23) (2011) 3990-4002.
14. Bach, R. et al, Changes in the spleen of channel catfish IctaZurus punctatus Rafinesque induced by infection with Aeromonas hydrophiZa., Journal of fish diseases 1(3) (1978) 205-217.
15. Bacharach, E. et al, Characterization of a Novel Orthomyxo-like Virus Causing Mass Die-Offs of Tilapia, mBio 7(2) (2016) e00431-16.
16. Baggiolini, M et al, Interleukin-8 and the chemokine family, International journal of immunopharmacology 17(2) (1995) 103-108.
17. Barreteau, H et al, Cytoplasmic steps of peptidoglycan biosynthesis, FEMS microbiology reviews 32(2) (2008) 168-207.
18. Baum, E Z, et al, Identification and characterization of new inhibitors of the Escherichia coli MurA enzyme, Antimicrob Agents Chemother 45(11) (2001) 3182-8.
19. Baumgartner, M I, et al, Experience of fish mortalities caused by epidemic clonal strains of Aeromonas hydrophila carrying a lysogenic bacteriophage, 2013.
20. Baumgartner, W A, et al, Lesions caused by virulent Aeromonas hydrophila in farmed catfish (Ictalurus punctatus and I. punctatus×I. furcatus) in Mississippi, J Vet Diagn Invest 29(5) (2017) 747-751.
21. Behera, B. et al, Emergence of tilapia lake virus associated with mortalities of farmed Nile tilapia Oreochromis niloticus (Linnaeus 1758) in India, Aquaculture 484 (2018) 168-174.
22. Bensen, D C, et al, Structure of MurA (UDP-N-acetylglucosamine enolpyruvyl transferase) from Vibrio fischeri in complex with substrate UDP-N-acetylglucosamine and the drug fosfomycin, Acta Crystallogr Sect F Struct Biol Cryst Commun 68(Pt 4) (2012) 382-5.
23. Bertani, G, STUDIES ON LYSOGENESIS 1.: The Mode of Phage Liberation by Lysogenic Escherichia coli, Journal of Bacteriology 62(3) (1951) 293-300.
24. Booth, N J, et al, Intracellular replication of Edwardsiella ictaluri in channel catfish macrophages., J Aquat Anim Health. 18 (2006) 101-108.
25. Brown, E D et al, MurA (MurZ), the enzyme that catalyzes the first committed step in peptidoglycan biosynthesis, is essential in Escherichia coli, J Bacteriol 177(14) (1995) 4194-7.
26. Cain, K. The many challenges of disease management in aquaculture, Journal of the World Aquaculture Society 53(6) (2022) 1080-1083.
27. Chen, P L, et al, Aeromonas dhakensis, an Increasingly Recognized Human Pathogen, Front Microbiol 7 (2016) 793-793.
28. Cho, Y. et al, Proteomic analysis of outer membrane proteins in Salmonella enterica Enteritidis, J Microbiol Biotechnol 25(2) (2015) 288-95.
29. Cipriano, R C, et al, Aeromonas hydrophila and motile aeromonad septicemias of fish, U.S. Fish and Wildfish Service, Fish Diseases Leaflet 681984.
30. Corbin, R. W. et al, Toward a protein profile of Escherichia coli: comparison to its transcription profile, Proc Natl Acad Sci USA 100(16) (2003) 9232-7.
31. Cuesta, A. et al, Total serum immunoglobulin M levels are affected by immunomodulators in seabream (Sparus aurata L.), Veterinary Immunology and Immunopathology 101(3) (2004) 203-210.
32. Curtiss, R 3rd, et al, New technologies in using recombinant attenuated Salmonella vaccine vectors, Crit Rev Immunol 30(3) (2010) 255-270.
33. Curtiss, R, et al, Salmonella enterica Serovar Typhimurium Strains with Regulated Delayed Attenuation In Vivo, Infection and Immunity 77(3) (2009) 1071-1082.
34. Curtiss, R, et al, Salmonella typhimurium deletion mutants lacking adenylate cyclase and cyclic AMP receptor protein are avirulent and immunogenic, Infection and Immunity 55(12) (1987) 3035-3043.
35. Curtiss, R. et al. Recombinant Edwardsiella bacterium, U.S. Pat. No. 9,045,742 (2015).
36. Dahal, N, et al, Tissue persistence and vaccine efficacy of tricarboxylic acid cycle and one-carbon metabolism mutant strains of Edwardsiella ictaluri, Vaccine 32(31) (2014) 3971-6.
37. Davison, J. Towards safer vectors for the field release of recombinant bacteria, Environ Biosafety Res 1(1) (2002) 9-18.
38. Del Pozo, J. et al, Syncytial Hepatitis of Tilapia (Oreochromis niloticus L.) is Associated With Orthomyxovirus-Like Virions in Hepatocytes, Veterinary Pathology 54(1) (2016) 164-170.
39. Dinh-Hung, N. et al, Dissecting the localization of Tilapia tilapinevirus in the brain of the experimentally infected Nile tilapia, Oreochromis niloticus (L.), Journal of Fish Diseases 44(8) (2021) 1053-1064.
40. Dong, H T, et al, Evidence of TiLV infection in tilapia hatcheries from 2012 to 2017 reveals probable global spread of the disease, Aquaculture 479 (2017) 579-583.
41. Dong, H T, et al, Experimental infection reveals transmission of tilapia lake virus (TiLV) from tilapia broodstock to their reproductive organs and fertilized eggs, Aquaculture 515 (2020) 734541.
42. Du, W et al, Two active forms of UDP-N-acetylglucosamine enolpyruvyl transferase in gram-positive bacteria, J Bacteriol 182(15) (2000) 4146-52.
43. Edwards, R A, et al, Improved allelic exchange vectors and their use to analyze 987P fimbria gene expression, Gene 207(2) (1998) 149-57.
44. Eyngor, M. et al, Identification of a Novel RNA Virus Lethal to Tilapia, Journal of Clinical Microbiology 52(12) (2014) 4137-4146.
45. Ferguson, H W, et al, Syncytial hepatitis of farmed tilapia, Oreochromis niloticus (L.): a case report, Journal of Fish Diseases 37(6) (2014) 583-589.
46. Fijan, N. et al, Some properties of the Epithelioma papulosum cyprini (EPC) cell line from carp cyprinus carpio, Annales de l'Institut Pasteur/Virologie 134(2) (1983) 207-220.
47. Galen, J E, et al, The delicate balance in genetically engineering live vaccines, Vaccine 32(35) (2014) 4376-85.
48. Gilbert, W, et al, Isolation of the lac repressor, Proceedings of the National Academy of Sciences of the United States of America 56(6) (1966) 1891-8.
49. Griffin, M J, et al, Edwardsiella piscicida identified in the Southeastern USA by gyrB sequence, species-specific and repetitive sequence-mediated PCR, Diseases of aquatic organisms 108(1) (2014) 23-35.
50. Griffin, M J, et al, Emergence of Edwardsiella piscicida in Farmed Channel ♀, Ictalurus punctatus×Blue ♂, Ictalurus furcatus, Hybrid Catfish Cultured in Mississippi, Journal of the World Aquaculture Society 50(2) (2019) 420-432.
51. Guan, Q, et al, Recombinant outer membrane protein A induces a protective immune response against Escherichia coli infection in mice, Applied Microbiology and Biotechnology 99(13) (2015) 5451-5460.
52. Guo, M et al, The Immune Adjuvant Effects of Flounder (Paralichthys olivaceus) Interleukin-6 on E. tarda Subunit Vaccine OmpV, Int J Mol Sci 18(7) (2017) 1445.
53. Hawke, J. Enteric septicemia of catfish, Southern Regional aquaculture Center (SRAC) Publication No. 477, 2015.
54. He, L. et al, Immunization of a novel outer membrane protein from Aeromonas hydrophila simultaneously resisting A. hydrophila and Edwardsiella anguillarum infection in European eels (Angullia angullia), Fish & Shellfish Immunology 97 (2020) 300-312.
55. Hemstreet, B, An update on Aeromonas hydrophila from a fish health specialist for summer 2010, Catfish Journal 24(4) (2010).
56. Hoelzer, K. et al, Vaccines as alternatives to antibiotics for food producing animals. Part 2: new approaches and potential solutions, Vet Res 49(1) (2018) 70.
57. Hossain, M J, et al, An Asian origin of virulent Aeromonas hydrophila responsible for disease epidemics in United States-farmed catfish, mBio 5(3) (2014) e00848-14.
58. Huicab-Pech, Z, et al, Pathogenic Bacteria in Oreochromis Niloticus Var. Stirling Tilapia Culture, Fisheries and Aquaculture Journal 8 (2017) 1-7.
59. Huizinga, H W, et al, Histopathology of red-sore disease (Aeromonas hydrophila) in naturally and experimentally infected largemouth bass Micropterus salmoides (Lacepede). Journal of fish diseases 2(4) (1979) 263-277.
60. Igbinosa, I H, et al, Emerging Aeromonas species infections and their significance in public health, ScientificWorldJournal 2012 (2012) 625023.
61. Ishihama, Y. et al, Protein abundance profiling of the Escherichia coli cytosol, BMC Genomics 9 (2008) 102.
62. Jespersen, M C, et al, BepiPred-2.0: improving sequence-based B-cell epitope prediction using conformational epitopes, Nucleic Acids Res 45(W1) (2017) W24-W29.
63. Jiang, X, et al, Immune effects of the vaccine of live attenuated Aeromonas hydrophila screened by rifampicin on common carp (Cyprinus carpio L), Vaccine 34(27) (2016) 3087-3092.
64. Jiang, Y, et al, Protection Against Necrotic Enteritis in Broiler Chickens by Regulated Delayed Lysis Salmonella Vaccines, Avian Dis 59(4) (2015) 475-85.
65. Juárez-Rodriguez, M D, et al, Live attenuated Salmonella vaccines displaying regulated delayed lysis and delayed antigen synthesis to confer protection against Mycobacterium tuberculosis, Infection and immunity 80(2) (2012) 815-31.
66. Kado, C, et al, Rapid procedure for detection and isolation of large and small plasmids, Journal of bacteriology 145(3) (1981) 1365-1373.
67. Kaiko, G E et al, Immunological decision-making: how does the immune system decide to mount a helper T-cell response?, Immunology 123(3) (2008) 326-38.
68. Kang, H Y, et al, Immune responses dependent on antigen location in recombinant attenuated Salmonella typhimurium vaccines following oral immunization, FEMS Immunology & Medical Microbiology 37(2-3) (2003) 99-104.
69. Kang, H Y, et al, Immune responses to recombinant pneumococcal PspA antigen delivered by live attenuated Salmonella enterica serovar typhimurium vaccine, Infection and immunity 70(4) (2002) 1739-49.
70. Karlin, S. et al, Characterizations of highly expressed genes of four fast-growing bacteria, J Bacteriol 183(17) (2001) 5025-40.
71. Katagiri, N W M et al, Locus of the Channel Catfish, IgH The, Journal of immunology (Baltimore, Md.: 1950) 169 (2002) 2488-2497.
72. Koesharyani, I. et al, Studi kasus infeksi tilapia lake virus (TiLV) pada ikan nila (Oreochromis niloticus), Jurnal Riset Akuakultur 13(1) (2018) 85-92.
73. Kong, Q. et al, Effect of deletion of genes involved in lipopolysaccharide core and O-antigen synthesis on virulence and immunogenicity of Salmonella enterica serovar typhimurium, Infection and immunity 79(10) (2011) 4227-39.
74. Kong, W, et al, Regulated programmed lysis of recombinant Salmonella in host tissues to release protective antigens and confer biological containment, Proceedings of the National Academy of Sciences 105(27) (2008) 9361-9366.
75. Kong, W, et al, Turning self-destructing <i>Salmonella</i> into a universal DNA vaccine delivery platform, Proceedings of the National Academy of Sciences 109(47) (2012) 19414-19419.
76. Konjufca, V, et al, Immunogenicity of recombinant attenuated Salmonella enterica serovar Typhimurium vaccine strains carrying a gene that encodes Eimeria tenella antigen S07, Infection and immunity 76(12) (2008) 5745-53.
77. Kordon, A O, et al, Effects of Live Attenuated Vaccine and Wild Type Strains of Edwardsiella ictaluri on Phagocytosis, Bacterial Killing, and Survival of Catfish B Cells, Frontiers in immunology 10 (2019) 2383-2383.
78. Kordon, A O, et al, Live attenuated Edwardsiella ictaluri vaccines enhance the protective innate immune responses of channel catfish B cells, Developmental & Comparative Immunology 109 (2020) 103711.
79. Kordon, A O, et al, Phagocytic and Bactericidal Properties of Channel Catfish Peritoneal Macrophages Exposed to Edwardsiella ictaluri Live Attenuated Vaccine and Wild-Type Strains, Front Microbiol 8 (2018) 2638-2638.
80. Kotton, C N et al, Enteric pathogens as vaccine vectors for foreign antigen delivery, Infection and immunity 72(10) (2004) 5535-47.
81. Lamers, C H J, Natural and acquired agglutinins to Aeromonas hydrophila in carp (Ciprinus carpio), Can. J. Fish. Aquat. Sci. 43(619-624) (1986).
82. Lavelle, E C et al, The role of TLRs, NLRs, and RLRs in mucosal innate immunity and homeostasis, Mucosal Immunol 3(1) (2010) 17-28.
83. LeBlanc, E V, et al, Hepatitis C Virus Glycan-Dependent Interactions and the Potential for Novel Preventative Strategies, Pathogens 10(6) (2021) 685.
84. Ledesma-Feliciano, C. et al, Improved DNA Vaccine Delivery with Needle-Free Injection Systems, Vaccines (Basel) 11(2) (2023) 280.
85. Lee, S et al, Recent advances of vaccine adjuvants for infectious diseases, Immune Netw 15(2) (2015) 51-7.
86. Leung, K Y, et al, Edwardsiella piscicida: A versatile emerging pathogen of fish, Virulence 10(1) (2019) 555-567.
87. Lewis, D H, et al, Drug-Induced Structural-Changes in Olfactory Organ of Channel Catfish Ictalurus-Punctatus, Rafinesque, J Fish Biol 26(3) (1985) 355-358.
88. Li, J. et al, Construction and Characterization of an Aeromonas hydrophila Multi-Gene Deletion Strain and Evaluation of Its Potential as a Live-Attenuated Vaccine in Grass Carp, Vaccines (Basel) 9(5) (2021) 451.
89. Li, L P, et al, Development of live attenuated Streptococcus agalactiae vaccine for tilapia via continuous passage in vitro, Fish Shellfish Immunol 45(2) (2015) 955-63.
90. López-Porras, A. et al, Genetic variability of Edwardsiella piscicida isolates from Mississippi catfish aquaculture with an assessment of virulence in channel and channel×blue hybrid catfish, J Fish Dis 44(11) (2021) 1725-1751.
91. Lund, V et al, Comparison of antibody responses in Atlantic cod (Gadus morhua L.) to Aeromonas salmonicida and Vibrio anguillarum, Developmental & Comparative Immunology 30(12) (2006) 1145-1155.
92. Ma, J. et al, A Review of Fish Vaccine Development Strategies: Conventional Methods and Modern Biotechnological Approaches, Microorganisms 7(11) (2019). Maiti, B, et al, Application of Outer Membrane Protein-Based Vaccines Against Major Bacterial Fish Pathogens in India, Frontiers in Immunology 11(1362) (2020).
93. Marsden, M J, et al, A live (ΔaroA) Aeromonas salmonicida vaccine for furunculosis preferentially stimulates T-cell responses relative to B-cell responses in rainbow trout (Oncorhynchus mykiss), Infection and immunity 64(9) (1996) 3863-3869.
94. Mondal, H et al, A review on the recent advances and application of vaccines against fish pathogens in aquaculture, Aquac Int 30(4) (2022) 1971-2000.
95. Muraille, E et al, Th1/Th2 Paradigm Extended: Macrophage Polarization as an Unappreciated Pathogen-Driven Escape Mechanism?, Frontiers in Immunology 5 (2014).
96. Muralinath, M, et al, Immunization with Salmonella enterica serovar Typhimurium-derived outer membrane vesicles delivering the pneumococcal protein PspA confers protection against challenge with Streptococcus pneumoniae, Infection and immunity 79(2) (2011) 887-94.
97. Nakayama, K. et al, Construction of an Asd⁺ expression-cloning Vector: stable maintenance and high level expression of cloned genes in a Salmonella vaccine strain, Nat Biotech 6(6) (1988) 693-697.
98. Newman, SG. Bacterial vaccines for fish., Annu. Rev. Fish Dis 3(145-185) (1993).
99. Odeyemi, O A, et al, Antibiotic resistance profiling and phenotyping of Aeromonas species isolated from aquatic sources, Saudi J Biol Sci 24(1) (2017) 65-70.
100. Oluwole, A O et al, Peptidoglycan biosynthesis is driven by lipid transfer along enzyme-substrate affinity gradients, Nat Commun 13(1) (2022) 2278.
101. Park, S B, et al, Pathogenesis of and strategies for preventing Edwardsiella tarda infection in fish, Veterinary research 43 (2012) 67.
102. Parra, D. et al, Evolution of B cell immunity, Annu Rev Anim Biosci 1 (2013) 65-97.
103. Parvathy, S. T. et al, Codon usage bias, Mol Biol Rep 49(1) (2022) 539-565.
104. Parvez, S, et al, Prediction of T and B Cell Epitopes in the Proteome of SARS-CoV-2 for Potential Use in Diagnostics and Vaccine Design, 2020.
105. Pascual, D W, et al, Oral bacterial vaccine vectors for the delivery of subunit and nucleic acid vaccines to the organized lymphoid tissue of the intestine, Behring Institute Mitteilungen (98) (1997) 143-52.
106. Peabody, M A, et al, PSORTm: a bacterial and archaeal protein subcellular localization prediction tool for metagenomics data, Bioinformatics 36(10) (2020) 3043-3048.
107. Pedersen, S. et al, Patterns of protein synthesis in E. coli: a catalog of the amount of 140 individual proteins at different growth rates, Cell 14(1) (1978) 179-90.
108. Pizarro-Cerdá, J, et al, The bacterial signal molecule, ppGpp, regulates Salmonella virulence gene expression, Molecular microbiology 52(6) (2004) 1827-44.
109. Plant, K P, et al, Advances in fish vaccine delivery, Developmental & Comparative Immunology 35(12) (2011) 1256-1262.
110. Plumb, J A, et al, Health maintenance and principal microbial diseases of cultured fishes, John Wiley & Sons 2010.
111. Preena, P G, et al, A peek into mass mortality caused by antimicrobial resistant Edwardsiella tarda in goldfish, Carassius auratus in Kerala, Biologia (Bratisl) 77(4) (2022) 1161-1171.
112. Pridgeon, J W, et al, Development and efficacy of novobiocin and rifampicin-resistant Aeromonas hydrophila as novel vaccines in channel catfish and Nile tilapia, Vaccine 29(45) (2011) 7896-904.
113. Pridgeon, J W, et al, Edwardsiella tarda and Aeromonas hydrophila isolated from diseased Southern flounder (Paralichthys lethostigma) are virulent to channel catfish and Nile tilapia, 2014.
114. Quandt, J, et al, Versatile suicide vectors which allow direct selection for gene replacement in gram-negative bacteria, Gene 127(1) (1993) 15-21.
115. Reddy, P N, et al, Functional characterization of a broad and potent neutralizing monoclonal antibody directed against outer membrane protein (OMP) of Salmonella typhimurium, Applied Microbiology and Biotechnology 104(6) (2020) 2651-2661.
116. Reed, L J et al, A SIMPLE METHOD OF ESTIMATING FIFTY PERCENT ENDPOINTS12, American Journal of Epidemiology 27(3) (1938) 493-497.
117. Robinson, H L, et al, Protection against a lethal influenza virus challenge by immunization with a haemagglutinin-expressing plasmid DNA, Vaccine 11(9) (1993) 957-60.
118. Roland, K, et al, Construction and evaluation of a delta cya delta crp Salmonella typhimurium strain expressing avian pathogenic Escherichia coli 078 LPS as a vaccine to prevent airsacculitis in chickens, Avian Dis 43(3) (1999) 429-41.
119. Rudorf, S. et al, Protein Synthesis in E. coli: Dependence of Codon-Specific Elongation on tRNA Concentration and Codon Usage, PLoS One 10(8) (2015) e0134994.
120. Samanta, M, et al, Molecular characterization of toll-like receptor 2 (TLR2), analysis of its inductive expression and associated down-stream signaling molecules following ligands exposure and bacterial infection in the Indian major carp, rohu (Labeo rohita), Fish & Shellfish Immunology 32(3) (2012) 411-425.
121. Sambrook, J, et al, Molecular Cloning; A Laboratory Manual, Cold Spring Harbor Press (2001).
122. Santander, J, et al, Fur-regulated iron uptake system of Edwardsiella ictaluri and its influence on pathogenesis and immunogenicity in the catfish host, Infection and immunity 80(8) (2012) 2689-2703.
123. Santander, J, et al, Inflammatory effects of Edwardsiella ictaluri lipopolysaccharide modifications in catfish gut, Infection and immunity 82(8) (2014) 3394-3404.
124. Santander, J, et al, Phenotype, virulence and immunogenicity of Edwardsiella ictaluri cyclic adenosine 3′, 5′-monophosphate receptor protein (Crp) mutants in catfish host, Fish & shellfish immunology 31(6) (2011) 1142-1153.
125. Santander, J, et al, The aspartate-semialdehyde dehydrogenase of Edwardsiella ictaluri and its use as balanced-lethal system in fish vaccinology, PloS one 5(12) (2010) e15944.
126. Santander, J. et al, Effects of cAMP receptor protein (Crp) gene in the fish pathogen Edwardsiella ictaluri, a potential strategy to develop live attenuated vaccines for the aquaculture industry. World Aquaculture Society, Aquaculture America annual meeting. Advances in Aquatic Veterinary Medicine. New Orleans, Louisiana, USA. (2011).
127. Shi, H, et al, Immunogenicity of a live recombinant Salmonella enterica serovar typhimurium vaccine expressing pspA in neonates and infant mice born from naïve and immunized mothers, Clinical and vaccine immunology: CVI 17(3) (2010) 363-71.
128. Shoemaker, C A, et al, Immersion vaccination with an inactivated virulent Aeromonas hydrophila bacterin protects hybrid catfish (Ictalurus punctatus×Ictalurus furcatus) from motile Aeromonas septicemia, Fish & Shellfish Immunology 82 (2018) 239-242.
129. Shotts, E B, et al, A medium for the selective isolation of Edwardsiella ictaluri, Journal of Wildlife Diseases 26(2) (1990) 214-218.
130. Siegrist, C. Vaccine immunology, in: S. A. Plotkin, et al (Eds.), Vaccines, Elsevier Inc.2013, pp. 14-32.
131. Sizemore, D R, et al, Attenuated Shigella as a DNA delivery vehicle for DNA-mediated immunization, Science (New York, N.Y.) 270(5234) (1995) 299-302.
132. Stillie, R et al, The functional significance behind expressing two IL-8 receptor types on PMN, Journal of leukocyte biology 86(3) (2009) 529-543.
133. Stratev, D, et al, Antimicrobial resistance of Aeromonas hydrophila isolated from different food sources: A mini-review, Journal of infection and public health 9(5) (2016) 535-44.
134. Subramaniam, K, et al, Genome Sequence of Tilapia Lake Virus Associated with Syncytial Hepatitis of Tilapia in an Ecuadorian Aquaculture Facility, Microbiol Resour Announc 8(18) (2019).
135. Subramaniam, K, et al, Megalocytivirus infection in cultured Nile tilapia Oreochromis niloticus, Diseases of aquatic organisms 119(3) (2016) 253-8.
136. Subramaniam, R. et al, Draft genome sequence of the basidiomycetous fungus Tinctoporellus epimiltinus strain RS1, Data in brief 23 (2019) 103796.
137. Sun, W. et al, Highly efficient method for introducing successive multiple scarless gene deletions and markerless gene insertions into the Yersinia pestis chromosome, Applied and environmental microbiology 74(13) (2008) 4241-5.
138. Swain, B, et al, Construction and Evaluation of Recombinant Attenuated Edwardsiella piscicida Vaccine (RAEV) Vector System Encoding Ichthyophthirius multifiliis (Ich) Antigen IAG52B, Front Immunol 12 (2021) 802760.
139. Swain, B, et al, NOD1 and NOD2 receptors in mrigal (Cirrhinus mrigala): Inductive expression and downstream signalling in ligand stimulation and bacterial infections, Journal of Biosciences 38(3) (2013) 533-548.
140. Swain, B, et al, Pathogenicity and immunogenicity of Edwardsiella piscicida ferric uptake regulator (fur) mutations in zebrafish, Fish & Shellfish Immunology 107 (2020) 497-510.
141. Swain, B. et al, Virulence, immunogenicity and live vaccine potential of aroA and phoP mutants of Edwardsiella piscicida in zebrafish, Microb Pathog 162 (2022) 105355.
142. Tamura, K. et al, MEGA6: Molecular Evolutionary Genetics Analysis version 6.0, Molecular biology and evolution 30(12) (2013) 2725-9.
143. Tanaka, T et al, IL-6 in inflammation, immunity, and disease, Cold Spring Harb Perspect Biol 6(10) (2014) a016295.
144. Tartor, Y H, et al, Virulotyping and genetic diversity of Aeromonas hydrophila isolated from Nile tilapia (Oreochromis niloticus) in aquaculture farms in Egypt, Aquaculture 541 (2021) 736781.
145. Tattiyapong, P. et al, Experimental infection of Tilapia Lake Virus (TiLV) in Nile tilapia (Oreochromis niloticus) and red tilapia (Oreochromis spp.), Veterinary microbiology 207 (2017) 170-177.
146. Tekedar, H C, et al, Draft Genome Sequence of Aeromonas hydrophila TN97-08, Genome announcements 4(3) (2016).
147. Tsheten, T, et al, An Outbreak of Aeromonas hydrophila Food Poisoning in Deptsang Village, Samdrup Jongkhar, Bhutan, 2016, J Res Health Sci 16(4) (2016) 224-227.
148. U.S.A.I.P.F. Center for Emerging Issues, Assessing Infectious Disease Emergence Potential in the U.S. Aquaculture Industry [electronic resource]: Phase 1, Veterinary Services, Center for Emerging Issues (2007).
149. U.S.D.A. Agricultural Statistics Board, Catfish Production, National Agricultural Statistics Service (NASS) (2020).
150. U.S. Dept. of Agriculture, Catfish Production: Value of Sales Up 12% from 2020, AgFax (2022).
151. Ulmer, J B, et al., Heterologous protection against influenza by injection of DNA encoding a viral protein, Science (New York, N.Y.) 259(5102) (1993) 1745-9.
152. Wang, Q, et al, Genome sequence of the versatile fish pathogen Edwardsiella tarda provides insights into its adaptation to broad host ranges and intracellular niches, PloS one 4(10) (2009) e7646-e7646.
153. Wang, S et al, New technologies in developing recombinant attenuated Salmonella vaccine vectors, Microb Pathog 58 (2013) 17-28.
154. Wang, S, et al, Salmonella vaccine vectors displaying delayed antigen synthesis in vivo to enhance immunogenicity, Infection and immunity 78(9) (2010) 3969-3980.
155. Wang, Y. et al, Quantitative proteomic analysis of iron-regulated outer membrane proteins in Aeromonas hydrophila as potential vaccine candidates, Fish & Shellfish Immunology 68 (2017) 1-9.
156. Weiner, J. H. et al, Proteome of the Escherichia coli envelope and technological challenges in membrane proteome analysis, Biochim Biophys Acta 1778(9) (2008) 1698-713.
157. Wieczorek, M et al, Major Histocompatibility Complex (MHC) Class I and MHC Class II Proteins: Conformational Plasticity in Antigen Presentation, Front Immunol 8 (2017) 292.
158. Wise, A L, et al, A Review of Bacterial Co-Infections in Farmed Catfish: Components, Diagnostics, and Treatment Directions, Animals (Basel) 11(11) (2021).
159. Wright, M. Mutants of Escherichia coli lacking endonuclease 1, ribonuclease 1, or ribonuclease 1l, Journal of Bacteriology 107(1) (1971) 87-94.
160. Xie, H X, et al, Edwardsiella tarda-Induced cytotoxicity depends on its type Ill secretion system and flagellin, Infection and immunity 82(8) (2014) 3436-3445.
161. Xu, D H, et al, Parasitism by protozoan Ichthyophthirius multifiliis enhanced invasion of Aeromonas hydrophila in tissues of channel catfish, Veterinary Parasitology 184(2) (2012) 101-107.
162. Xu, J et al, IgM and IgD heavy chains of yellow catfish (Pelteobagrus fulvidraco): Molecular cloning, characterization and expression analysis in response to bacterial infection, Fish & Shellfish Immunology 84 (2019) 233-243.
163. Xu, W. et al, Edwardsiella piscicida virulence effector trxlp promotes the NLRC4 inflammasome activation during infection, Microbial Pathogenesis 123 (2018) 496-504.
164. Yadav, D K, et al, Chapter 26—Vaccines: Present Status and Applications, in: A. S. Verma, et al (Eds.), Animal Biotechnology, Academic Press, San Diego, 2014, pp. 491-508.
165. Yamkasem, J. et al, Evidence of potential vertical transmission of tilapia lake virus, Journal of Fish Diseases 42(9) (2019) 1293-1300.
166. Yu, C S, et al, CELLO2GO: a web server for protein subCELlular LOcalization prediction with functional gene ontology annotation, PLoS One 9(6) (2014) e99368-e99368.
167. Zeng, W. et al, Cell Culture-Derived Tilapia Lake Virus-Inactivated Vaccine Containing Montanide Adjuvant Provides High Protection against Viral Challenge for Tilapia, Vaccines (Basel) 9(2) (2021) 86.
168. Zhang, D. et al, Detection and quantification of virulent Aeromonas hydrophila in channel catfish tissues following waterborne challenge, FEMS Microbiology Letters 363(9) (2016).
169. Zhang, D. et al, The severity of motile Aeromonas septicemia caused by virulent Aeromonas hydrophila in channel catfish is influenced by nutrients and microbes in water, Aquaculture 519 (2020) 734898.
170. Zhang, G et al, Toll-like receptor-mediated NF-kappaB activation: a phylogenetically conserved paradigm in innate immunity, J Clin Invest 107(1) (2001) 13-9.
171. Zhang, Q. et al, The Edwardsiella piscicida Type III Effector EseJ Suppresses Expression of Type 1 Fimbriae, Leading to Decreased Bacterial Adherence to Host Cells, Infection and immunity 87(7) (2019) e00187-19.
172. Zhang, W. et al, Protective immune responses of recombinant outer membrane proteins OmpF and OmpK of Aeromonas hydrophila in European eel (Anguilla anguilla), Aquaculture Research 50(12) (2019) 3559-3566.
173. Zhou, P. et al, Phenotype, Virulence and Immunogenicity of Edwardsiella piscicida Cyclic AMP Receptor Protein (Crp) Mutants in Catfish Host, Microorganisms 8(4) (2020).
174. Zhou, Z. et al, Codon usage is an important determinant of gene expression levels largely through its effects on transcription, Proc Natl Acad Sci USA 113(41) (2016) E6117-e6125.
175. Zou, J. et al, The Function of Fish Cytokines, Biology (Basel) 5(2) (2016) 23.

1. A recombinant, attenuated, biologically-contained Edwardsiella bacterial vector displaying regulated delayed lysis attributes, the vector comprising a gene encoding at least one antigen from a fish pathogen;
wherein the at least one antigen or a DNA vaccine vector containing the gene encoding the at least one antigen induces expression of host genes for enhancement of innate immunity after entry of the antigen or DNA vaccine vector into a eukaryotic host cell.

2. The recombinant attenuated biologically contained Edwardsiella bacterial vector of claim 1, wherein the bacterial vector comprises an Edwardsiella piscicida strain.

3. (canceled)

4. The recombinant attenuated biologically contained Edwardsiella bacterial vector of claim 1, wherein entry into the eukaryotic cell causes expression of one or more host cytokine genes selected from an il-8 gene, an il-1β gene, a tnf-α gene, an il-6 gene, and an ifn-γ gene.

5.-6. (canceled)

7. The recombinant attenuated biologically contained Edwardsiella bacterial vector of claim 1, wherein entry into a eukaryotic cell causes expression of genes encoding CD4-1, CD4-2, CD8-α, CD8-β, MHC-II, or any combination thereof.

8. The recombinant attenuated biologically contained Edwardsiella bacterial vector of claim 1, wherein the fish pathogen comprises a bacterial pathogen, a viral pathogen, or a parasitic pathogen.

9.-10. (canceled)

11. The recombinant attenuated biologically contained Edwardsiella bacterial vector of claim 8, wherein the bacterial pathogen comprises Aeromonas hydrophila.

12. The recombinant attenuated biologically contained Edwardsiella bacterial vector of claim 9, wherein the viral pathogen comprises tilapia lake virus (TiLV).

13. The recombinant attenuated biologically contained Edwardsiella bacterial vector of claim 11, wherein the antigen comprises outer membrane protein CCO02501, AGM43135, AGM42919, AOKFG8, AOKGW8, AOKQ46, AOKQZ1, AOKIU8, AGH12866, a TonB domain of AOKFG8, an OMP domain of AOKFG8, a Plug domain of AOKFG8, or any combination thereof.

14. The recombinant attenuated biologically contained Edwardsiella bacterial vector of claim 12, wherein the TiLV gene encoding the at least one antigen and has at least 80% sequence identity with any one of SEQ ID NOs. 47-55.

15. The recombinant attenuated biologically contained Edwardsiella bacterial vector of claim 1, wherein the gene encoding the at least one antigen is incorporated into a plasmid.

16. (canceled)

17. The recombinant attenuated biologically contained Edwardsiella bacterial vector of claim 1, wherein the recombinant vector transiently colonizes at least one tissue type in a subject, and wherein colonization by the vector induces release of the at least one antigen.

18. (canceled)

19. The recombinant attenuated biologically contained Edwardsiella bacterial vector of claim 18, wherein in the vector, at least one gene promoter has been replaced with an araC P_{araBAD} cassette to create a delayed attenuation phenotype.

20. The recombinant attenuated biologically contained Edwardsiella bacterial vector of claim 19, wherein the at least one gene promoter comprises a promoter for the fur gene, the crp gene, or both the fur and crp genes.

21. The recombinant attenuated biologically contained Edwardsiella bacterial vector of claim 19, wherein presence of the araC P_{araBAD} cassette induces vector dependence on arabinose.

22.-23. (canceled)

24. A vaccine comprising the recombinant attenuated biologically contained Edwardsiella bacterial vector of claim 1.

25. A method for generating an immune response to at least one pathogen in a subject, the method comprising administering to a subject the vaccine of claim 24 to the subject.

26. (canceled)

27. The method of claim 25, wherein the subject is a fry, juvenile, adult, or spawning fish in an aquaculture system.

28. The method of claim 27, wherein the subject is a tilapia, catfish, trout, mullet, salmon, carp, or striped bass.

29. The method of claim 25, wherein the pathogen comprises tilapia lake virus (TiLV) or Aeromonas hydrophila.

30.-32. (canceled)

33. The method of claim 25, wherein administration is carried out by bath immersion of the subject or wherein the vaccine is administered by intracelomic (i.e.) injection of the subject.

34.-47. (canceled)
